(19) United States
(12) Patent Application Publication
(10) Pub. No.: US 2002/0082264 A1
(43) Pub. Date: Jun. 27, 2002

(54) Medicaments for viral diseases

(76) Inventors: Susanne Nikolic, Wuppertal (DE); Stephan Bartel, Kurten (DE); Michael Brands, Wuppertal (DE); Ulrich Niewohner, Wermelskirchen (DE); Arnold Paessens, Haan (DE); Erwin Graef, Wulfrath (DE); Karl-Heinz Schlemmer, Wuppertal (DE); Kerstin Henninger, Wuppertal (DE); Rainer Endermann, Wuppertal (DE); Olaf Weber, Woodbridge, CT (US); Diana Koletzki, Wuppertal (DE)

Correspondence Address: Jeffrey M. Greenman, Vice President, Patents and Licensing, Bayer Corporation, 400 Morgan Lane, West Haven, CT, 06516, US

(21) Appl. No.: 09/943,046

(22) Filed: Aug. 30, 2001

(30) Foreign Application Priority Data
Sep. 6, 2000 (DE) 10043791.5
Nov. 6, 2000 (DE) 10054932.2

(51) Int. Cl. A61K 31/496; A61K 31/505; A61K 31/4709; A61K 31/4025; A61K 31/4433; A61K 31/352

(52) U.S. Cl. 514/254.11; 514/256; 514/422; 514/314; 514/337; 514/454; 514/456

(57) ABSTRACT

Chromanone derivatives are highly active antiviral agents. Combinations of chromanones and/or chromanols with HBV polymerase inhibitors, HBV DNA inhibitors or HBV core protein inhibitors and/or isoxazoles and, where appropriate, interferon inhibit the replication of HBV viruses better than agents disclosed previously.

The present invention relates to the use of substituted chroman-4-one derivatives for producing antiviral medicaments, in particular for the treatment and prophylaxis of HBV infections, to novel chroman-4-one derivatives and to processes for their preparation. The invention further relates to combinations of A) non-nucleosidic inhibitors from the class of chromanones or chromanols with B) other HBV-antiviral active substances such as (i) nucleoside analogues such as, for example, lamivudine, where appropriate (ii) HBV DNA inhibitors or HBV core protein inhibitors such as dihydropyrimidines and/or (iii) isoxazoles, and, where approprriate, C) immuno-modulators such as, for example interferon, to a process for producing these combinations and their use as medicaments, in particular for the treatment and prophylaxis of HBV infections. The combinations thus comprise at least two, three or four active substances of groups A and B.

“Combinations” mean for the purpose of the invention not only dosage forms which contain all the components (so-called fixed combinations), and combination packs which contain the components separate from one another, but also components which are administered simultaneously or sequentially, as long as they are employed for the treatment or prophylaxis of the same disease.

The hepatitis B virus belongs to the family of hepadna viruses. It causes an acute and/or a persistant/progressive chronic disease. Many other clinical manifestations in the pathological state are also caused by the hepatitis B virus—in particular chronic inflammation of the liver, cirrhosis of the liver and hepatocellular carcinoma. In addition, coinfection with the hepatitis delta virus may have adverse effects on the progress of the disease.

Several possibilities have already been proposed for virus inhibition:

1. inhibition of the virus by dihydropyrimidines which bring about a large reduction in the viral DNA and in the viral core protein;

2. inhibition of HBV polymerase by analogues of the substrates of this enzyme such as lamivudine, FTC, adefovir dipivoxil, abacavir, β-L-FDDC, L-FMAU (clevudine) and BMS-200475 (entecavir);

3. inhibition of HBV by immunological principles such as, for example, the treatment of chronic hepatitis by interferon;

4. inhibition by other active substances whose modes of action are not known or are the subject of speculation, such as, for example, AT-61 N-[(1E)-2-chloro-2-phenyl-1-(1-piperidinylcarbonyl)ethenyl]benzamide, which evidently intervenes in the process of packaging the pregenomic RNA into the incomplete core particles; cf. King et al., Antimicrob. Agents and Chemother. 42, 3179-3186 (1998);

5. stimulation of the host's immune defences, such as, for example, with thymosin-α.

The only agents approved for the treatment of chronic hepatitis are interferon and lamivudine. However, interferon has only moderate activity and has unwanted side effects; although lamivudine has good activity, resistance develops rapidly during treatment and a rebound effect occurs in most cases after discontinuation of the therapy.

Therapeutic agents employed to date for the treatment of HBV-infected patients, such as, for example, interferon or lamivudine, are employed as monotherapy. It is known from clinical studies that combinations of the two inhibitors have no advantage for controlling HBV diseases.

Novel agents for a tolerated and effective therapy are therefore desirable.

It has been found, surprisingly, that chromanone derivatives are highly effective for hepatitis viruses.

The invention relates to the use of compounds of the formula

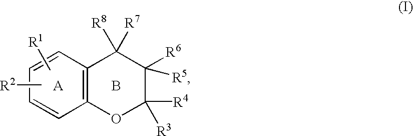

in which

R¹ denotes bromine, phenyl, pyrrolyl, pyridyl, pyrimidinyl, piperazinyl or quinolinyl, of which the cyclic substituents may each be substituted up to three times, identically or differently, by halogen, C₁-C₆-alkyl, trifluoromethyl, C₁-C₆-alkoxy, C₁-C₆-alkoxycarbonyl, C₁-C₆-alkylthio, nitro, cyano, amino, aminocarbonyl or benzyloxycarbonylamino,

R² denotes hydrogen or

R¹ and R² together with two adjacent carbon atoms of ring A denote a fused-on benzene ring,

R³ and R⁴ denote, independently of one another, linear or branched C₁-C₆-alkyl which may be substituted by carboxyl, C₁-C₄-alkoxy and/or C₁-C₄-alkoxy-carbonyl, or

R³ and R⁴ together denote an optionally C₁-C₄-alkoxycarbonyl-substituted C₂-C₇-alkylene radical in which one CH₂ group may be replaced by an oxygen atom or by NR⁹ (with R⁹=hydrogen, benzoyl or benzyloxycarbonyl), or together with the carbon atom on which they are located denote a tetrahydro-2H-pyran ring,

R⁵ denotes linear or branched C₂-C₆-alkyl which may be substituted by cyano, C₁-C₄-alkoxycarbonyl, carboxyl or aminocarbonyl,

R⁶ denotes hydrogen or linear or branched C₂-C₆-alkyl which may be substituted by cyano, C₁-C₄-alkoxycarbonyl, carboxyl or aminocarbonyl,

R⁷ denotes hydrogen,

R⁸ denotes hydroxyl or

R⁷ and R⁸ together denote an oxo group,

for the production of antiviral medicaments in particular for the treatment and prophylaxis of HBV infections.

Compounds to be preferably used according to the invention correspond to the formula I in which

R¹ denotes phenyl, pyridyl or quinolinyl, each of which can be substituted up to three times by fluorine or chlorine or once by methyl, trifluoromethyl, methoxy, methylthio, nitro, cyano or amino; pyrrolyl which may be substituted by tert-butoxycarbonyl;

R² denotes hydrogen,

R³ and R⁴ each denotes methyl or

R³ and R⁴ together denote a C₃-C₅-alkylene radical in which one CH₂ group may be replaced by an oxygen atom, or together with the carbon atom on which they are located denote a tetrahydro-2H-pyran ring,

R⁵ denotes cyanoethyl,

R⁶ denotes hydrogen or cyanoethyl, and

R⁷ and R⁸ have the meanings indicated above.

Compounds to be particularly preferably used according to the invention correspond to the formula I in which

R¹ denotes phenyl which may be substituted up to three times by fluorine, up to twice by chlorine or once by methyl, trifluoromethyl, methoxy, methylthio, nitro, cyano or amino; pyridyl; pyrrolyl, which may be substituted by tertbutoxycarbonyl;

R² denotes hydrogen,

R³ and R⁴ each denotes methyl or

R³ and R⁴ together denote a C₃-C₅-alkylene radical or together with the carbon atom on which they are located denote a tetrahydro-2H-pyran ring,

R⁵ and R⁶ denote cyanoethyl, and

R⁷ and R⁸ have the meanings indicated above.

The compounds to be used according to the invention which are most effective against HBV are those of Examples 76, 82, 97, 99, 116 and 132.

Alkyl within the framework of the chromanone/chromanol definition represents a straight-chain or branched alkyl radical having 1 to 6, preferably 1 to 4, carbon atoms, such as, for example, methyl, ethyl, propyl, isopropyl, isobutyl, tert-butyl, n-pentyl and n-hexyl.

Alkoxy within the framework of the chromanone/chromanol definition represents a straight-chain or branched alkoxy radical having 1 to 6, preferably 1 to 4, carbon atoms, such as, for example, methoxy, ethoxy, propoxy, isopropoxy, isobutoxy, tert-butoxy, n-pentoxy and n-hexoxy.

Alkylthio within the framework of the chromanone/chromanol definition represents a straight-chain or branched alkoxythio radical having 1 to 6, preferably 1 to 4, carbon atoms, such as, for example, methylthio, ethylthio and propylthio.

Alkoxycarbonyl within the framework of the chromanone/chromanol definition represents a straight-chain or branched alkoxycarbonyl radical having 1 to 6, preferably 1 to 4, carbon atoms, such as, for example, methoxycarbonyl, ethoxycarbonyl, propoxycarbonyl, isopropoxycarbonyl, isobutoxycarbonyl, tert-butoxycarbonyl, n-pentoxycarbonyl and n-hexoxycarbonyl.

Alkylene within the framework of the chromanone/chromanol definition represents C₂-C₇-, preferably C₃-C₅-alkylene such as, for example, ethylene, 1,3-propylene, 1,4-butylene, 1,5-pentylene, 1,6-hexylene and 1,7-heptylene.

Halogen within the framework of the chromanone/chromanol definition represents fluorine, chlorine, bromine or iodine.

The compounds I to be used according to the invention are for the most part disclosed in EP-A 4 624 (=U.S. Pat. No. 4,261,988). They were recommended therein because of their hypocholesteraemic activity and their growth-promoting action; no antiviral action is, however, described therein.

Many chroman-4-ones are known; compare German Offenlegungsschrift 26 11 910 and the volume in the series “Heterocyclic Compounds” “Chromenes, Chromanones and Chromones”, page 207 et seq., edited by G. P. Ellis, New York, London, Sydney, Toronto 1977. They can be obtained by the processes described therein or analogous ones.

Thus, chroman-4-ones can be prepared, for example, by

a) condensation of o-hydroxyacetophenone with carbonyl compounds in the presence of basic condensing agents,

b) Fries rearrangement of the phenol esters of α,β-unsaturated carboxylic acids,

c) cyclization of β-phenoxypropionic acids with acidic condensing agents or

d) reduction of 4H-chromen-4-ones;

compare, for example, also H. J. Kabbe in Synthesis 1978, 886-889.

The substitution of ring A of the chroman-4-one structure can be obtained by Suzuki-reaction (compare Chem. Rev. 90, 879 (1990), Pure Appl.Chem. 63, 419 (1991) and Chem. Rev. 1995, 2457-2483) of the correspeonding organoboron compounds with chromanones which are substituted in ring A by halogen, pseudohalogen, aryldiazonium salt or trifluoromethylsulphonate, or by Stille or Migita-Stille-Kosugi coupling; compare, for example, Angew. Chem. Int. Ed. Engl. 25, 508 (1986), Pure Appl. Chem. 57, 1771 (1985), Synthesis 1987, 693-696 and 1992, 803, Tetrahedron Lett. 27, 4407-4410 (1986). Conversely, it is also possible to react chromanone boron or chromanone-stannane compounds with halogeno-, pseudohalogen-, aryldiazonium salt- or trifluoromethylsulphonate-aryls or -hetaryls.

It is, of course, also possible to start directly from the appropriately substituted phenols or phenol ethers or esters, that is to say to carry out the arylation at the stage of the chromanone precursors.

The amount of the organoboron compound employed for the Suzuki reaction can be 1 to 10, preferably 1 to 2, mol, based on halogen-, pseudohalogen-, aryldiazonium salt- or trifluoromethylsulphonate-substituted chromanone.

Catalysts preferred for the Suzuki reaction and the Stille or Migita-Kosugi-Stille coupling comprise, for example, the following palladium compounds: palladium(II) acetate, tetrakis(triphenylphosphine)palladium(0), tri-p-tolylphosphine-palladium(II) acetate, tetrakis(triphenylphosphine)palladium(0), trans-dichlorobis(tri-o-tolylphosphine)palladium (II), benzyl-bis-(triphenylphosphine)chloride, bis(triphenylphosphine)palladium(II) chloride and [1,1′-bis(diphenylphosphino)ferrocene]palladium(II) chloride. The amount of catalysts employed may vary within wide limits; amounts of from 1 to 10, preferably 1.5 to 8, mol % based on halogen-, pseudohalogen-, aryldiazonium salt- or trifluoromethylsulphonate-substituted chromanone have generally proved suitable.

Bases preferred for the Suzuki reaction comprise, for example, alkali metal and thallium hydroxides, carbonates, bicarbonates, halides and phosphates such as potassium and sodium hydroxide, caesium fluoride, potassium, sodium, caesium and thallium carbonate, sodium bicarbonate, potassium phosphate, C₁-C₆-alkylammonium halides such as tert.-butylammonium fluoride, alkali metal alcoholates and phenolates such as sodium ethoxide (with or without tert-butylammonium fluoride and crown ether) and amines, preferably tertiary amines such as triethylamine. The amount of bases employed may vary within wide limits; amounts of from 1 to 2, preferably from 1.1 to 1.8, in particular 1.1 to 1.6, mol per mole of starting chromanone are generally sufficient; bases such as, for example, triethylamine may, however, also serve as solvents and, in this case, are employed in large excess.

Solvents preferred for the Suzuki reaction and the Stille or Migita-Kosugi-Stille coupling comprise aromatic compounds such as benzene, toluene, halogenohydrocarbons such as dichloromethane, aliphatic alcohols such as ethanol, ethers such as diethyl ether, tetrahydrofuran, dioxane, dimethoxyethane, nitriles such as acetonitrile, amides such as dimethylformamide, and water, and mixtures thereof, such as, for example, toluene/ethanol, benzene/ethanol and acetonitrile/water.

The Suzuki and the Stille or Migita-Kosugi-Stille C—C coupling can be carried out at temperatures of from 20 to 150, preferably 40 to 120,° C. and in the presence of palladium(0). The reaction can be illustrated, for example, by the diagram

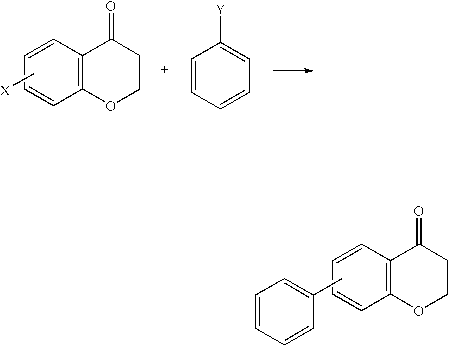

X=halogen, pseudohalogen, aryldiazonium salt, trifluoromethylsulphonate

Y=stannane, boron organyl.

It is also possible for X and Y in this reaction diagram to be transposed.

The ligand frequently used for Pd(0) is triphenylphosphine or its mono(sodium sulphonate) derivative. From 0.8 to 1.3 mol of stannane are preferably employed per mole of halogen-, pseudohalogen-, aryldiazonium salt- or trifluoromethylsulphonate-substituted chromanone. The abovementioned Pd(0) catalysts can be employed for the catalysis.

The substituents in position 3 can be introduced by Michael addition of α,β-ethylenically unsaturated compounds onto chromanones unsubstituted in position 3 in the presence of bases, as disclosed, for example, in EP-A 4 624. The α,β-ethylenically unsaturated compounds particularly preferred for the Michael addition are alkyl acrylates and acrylonitrile. The amount of α,β-ethylenically unsaturated compound employed can be from 1 to 8, preferably 1 to 5, mol per mole of chromanone starting compound.

Bases preferred for the Michael addition comprise sodium and potassium hydride, alkali metal C₁-C₄-alcoholates (in particular sodium alcoholates), lithium bis(trimethylsilyl)amide (Tetrahedron Lett. 35, 6347-6350 (1994)) and lithium N,N-diisopropylamide (J. Chem. Soc. Perkin Trans.I 1995, 197-201, and J. Am. Chem. Soc. 113, 2071-2092 (1991)). The amount of base employed can be from 0.4 to 10, preferably 1 to 3, mol per mole of chromanone starting compound.

Solvents preferred for the Michael addition comprise, inter alia, ethers such as tetrahydrofuran and dioxane, and alcohols, with preference being given, on use of alkali metal C₁-C₄-alcoholates as base, to the corresponding alcohols as solvent.

The Michael addition can be carried out at temperatures from 20 to 100, preferably 40 to 80,° C.

In place of this Michael addition it is also possible to use the corresponding Mukaiyama reaction (with titanium compounds instead of base catalysis); compare, for example, Chem. Lett. 1987, 743-746, Bull. Chem. Soc. Jpn. 49, 779-783 (1976), Angew. Chem. 111, 3574-3576 (1999), Synthesis 1977, 91-110. In addition, the substituents R⁵/R⁶ can also be prepared by reacting the chromanones unsubstituted in position 3 with halogen compounds; compare also Tetrahedron Lett. 1978, 573-576, J. Org. Chem. 61, 2081-2084 (1996).

The chromanol compounds according to the invention can be obtained by hydrogenation of the corresponding chromanones, for example with complex hydrides such as sodium borohydride.

The invention further relates to novel compounds of the above formula I in which

R¹ denotes bromine, phenyl, pyrrolyl, pyridyl, pyrimidinyl, piperazinyl or quinolinyl, of which the cyclic substituents may in each case be substituted up to three times, identically or differently, by halogen, C₁-C₆-alkyl, trifluoromethyl, C₁-C₆-alkoxy, C₁-C₆-alkoxycarbonyl, C₁-C₆-alkylthio, nitro, cyano, amino, aminocarbonyl or benzyloxycarbonylamino, but where phenyl must have at least one substituent,

R² denotes hydrogen,

R³ and R⁴ denote, independently of one another, linear or branched C₁-C₆-alkyl which may be substituted by carboxyl, C₁-C₄-alkoxy and/or C₁-C₄-alkoxycarbonyl or

R³ and R⁴ together denote an optionally C₁-C₄-alkoxycarbonyl-substituted C₂-C₇-alkylene radical in which one CH₂ group may be replaced by an oxygen atom or by NR⁹ (with R⁹=hydrogen, benzoyl or benzyloxycarbonyl), or together with the carbon atom on which they are located denote a tetrahydro-2H-pyran ring,

R⁵ denotes linear or branched C₂-C₆-alkyl which may be substituted by cyano, C₁-C₄-alkoxycarbonyl, carboxyl or aminocarbonyl,

R⁶ denotes hydrogen or linear or branched C₂-C₆-alkyl which may be substituted by cyano, C₁-C₄-alkoxycarbonyl, carboxyl or aminocarbonyl,

R⁷ denotes hydrogen,

R⁸ denotes hydroxyl or

R⁷ and R⁸ together denote an oxo group.

Preferred compounds according to the invention correspond to the formula I in which

R¹ denotes phenyl, pyrrolyl, pyridyl or quinolinyl, each of which may be substituted up to three times by fluorine or chlorine or once by methyl, trifluoromethyl, methoxy, methylthio, tert-butoxycarbonyl, nitro, cyano or amino, but where phenyl must have at least one substituent,

R² denotes hydrogen,

R³ and R⁴ each denotes methyl or

R³ and R⁴ together denote a C₃-C₅-alkylene radical in which one CH₂ group may be replaced by an oxygen atom, or together with the carbon atom on which they are located denote a tetrahydro-2H-pyran ring,

R⁵ denotes cyanoethyl,

R⁶ denotes hydrogen or cyanoethyl, and

R⁷ and R⁸ have the meanings indicated above.

Particularly preferred compounds according to the invention correspond to formula I in which

R¹ denotes phenyl which may be substituted up to three times by fluorine, up to twice by chlorine or once by methyl, trifluoromethyl, methoxy, methylthio, nitro, cyano or amino, but where phenyl must have at least one substituent; pyridyl; pyrrolyl which may be substituted by tert-butoxycarbonyl;

R² denotes hydrogen,

R³ and R⁴ each denotes methyl or

R³ and R⁴ together denote a C₃-C₅-alkylene radical or together with the carbon atom on which they are located denote a tetrahydro-2H-pyran ring,

R⁵ and R⁶ denote cyanoethyl, and

R⁷ and R⁸ have the meanings indicated above.

The most preferred compounds are those of Examples 76, 82, 97, 99, 116 and 132.

The invention further relates to a process for preparing the novel compounds, in which either

A) compounds of the formula

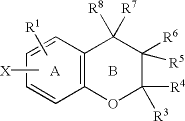

in which

R¹ to R⁸ have the meanings indicated above, and

X represents halogen, preferably bromine, pseudohalogen, aryldiazonium salt or trifluoromethylsulphonate,

are subjected with compounds of the formula R²B(OH)₂ to a Suzuki reaction or

are subjected with compounds of the formula R²SnR₃ (in which R² has the meaning indicated above, and R denotes alkyl) to a Stille or a Migita-Stille-Kosugi coupling, or

B) compounds of the formula

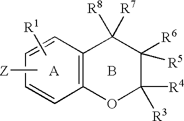

in which

R¹ to R⁸ have the meanings indicated above, and

Z represents —B(OH)₂ or —SnR₃, where R has the meaning indicated under A),

are reacted with compounds of the formula R²X where X has the meaning indicated under A), in a Stille or a Migita-Stille-Kosugi coupling, or

C) compounds of the formula

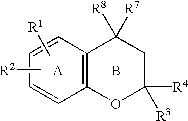

in which

R¹ to R⁸ have the meanings indicated above,

are reacted with α,β-unsaturated compounds suitable for introducing the substituents R⁵ and/or R⁶ in a Michael addition, and where appropriate

D) the reaction product from step A), B) or C) is hydrogenated to the corresponding chromanol.

It has additionally been found that combinations of A) non-nucleosidic inhibitors from the class of chromanones or chromanols, B) HBV-antiviral active substances different from A and, where appropriate, C) immunomodulators such as, for example, interferon do not have or have only some of the disadvantages of the state of the art.

The invention therefore relates to combinations of

A) at least one chromanone and/or chromanol,

B) at least one HBV-antiviral active substance different from A, preferably (i) at least one HBV polymerase inhibitor, where appropriate combined with an HBV-antiviral active substance different from A and B(i), in particular (ii) an HBV DNA inhibitor or HBV core protein inhibitor and/or (iii) an isoxazole and, where appropriate,

C) at least one immunomodulator.

The invention thus relates to combinations of nucleosidic and non-nucleosidic inhibitors and, where appropriate, immunomodulators for the treatment and prophylaxis of HBV infections, and to the use of these combinations for the treatment of HBV-induced diseases.

It was not predictable that the combinations according to the invention inhibit the replication of the HBV virus considerably better than the agents known from the state of the art or their known combinations. The use of the combinations according to the invention for the treatment of HBV-induced diseases provides valuable advantages compared with monotherapy with the individual compounds, namely principally a synergistic antiviral activity, but also good tolerability of the combinations according to the invention in the range of toxicity at which 50% of cells survive (“tox-50”)-compared with the tox-50 of the individual components.

Preferred chromanones and chromanols A comprise the compounds of the formula (I) described above.

The substances referred to as HBV polymerase inhibitors B(i) for the purposes of the invention are those which, in the endogenous polymerase assay (Ph. A. Furman et al. in Antimicrobial Agents and Chemotherapy, Vol. 36 (No. 12), 2688 (1992)) lead to an inhibition of the formation of an HBV DNA double strand, so as to result in a maximum of 50% of the activity of the non-inhibited sample:

Preferred HBV polymerase inhibitors B(i) comprise, for example,

3TC=lamivudine=4-amino-1-[(2R-cis)-2-(hydroxymethyl)-1,3-oxathiolan-5-yl]-pyrimidin-2(1H)-one, compare European Patent Specification 382 526 (=U.S. Pat. No. 5,047,407) and WO 91/11186 (U.S. Pat. No. 5,204,466);

adefovir dipivoxil=9-{2-[[bis[(pivaloyloxy)-methoxy]-phosphinyl]-methoxy]-ethyl}-adenine, compare European Patent Specification 481 214 (=U.S. Pat. Nos. 5,663,159 and 5,792,756), U.S. Pat. Nos. 4,724,233 and 4,808,716;

BMS-200475=[1 S-(1α,3α,4β)]-2-amino-1,9-dihydro-9-[4-hydroxy-3-(hydroxymethyl)-2-methylene-cyclopentyl]-6H-purin-6-one, compare European Patent Specification 481 754 (=U.S. Pat. No. 5,206,244 and 5,340,816), WO 98/09964 and 99/41275;

abacavir=(−)-(1S-cis)-4-[2-amino-6-(cyclopropylamino)-9H-purin-9-yl]-2-cyclopentene-1-methanol, compare European Patent Specification 349 242 (=U.S. Pat. No. 5,049,671) and European Patent Specification 434 450 (=U.S. Pat. No. 5,034,394);

FTC=(2R-cis)-4-amino-5-fluoro-1-[2-(hydroxymethyl)-1,3-oxathiolan-5-yl]-pyrimidin-2(1H)-one, compare WO 92/14743 (=U.S. Pat. Nos. 5,204,466, 5,210,085, 5,539,116, 5,700,937, 5,728,575, 5,814,639, 5,827,727, 5,852,027, 5,892,025, 5,914,331, 5,914,400) and WO 92/18517;

β-L-FDDC=5-(6-amino-2-fluoro-9H-purin-9-yl)-tetrahydro-2-furanmethanol, compare WO 94/27616 (=U.S. Pat. Nos. 5,627,160, 5,561,120, 5,631,239 and 5,830,881);

L-FMAU 1-(2-deoxy-2-fluoro-β-L-arabinofuranosyl)-5-methyl-pyrimidin-2,4(1H, 3H)-dione, compare WO 99/05157, WO 99/05158 and U.S. Pat. No. 5,753,789.

HBV DNA inhibitors and HBV core protein inhibitors B(ii) are those non-nucleosidic inhibitors which show intra- and extracellular inhibition of HBV DNA and at least halve the half-life of the HBV core protein in the cell.

Preferred HBV DNA inhibitors and HBV core protein inhibitors B(ii) are, for example, dihydropyrimidines, preferably those described in German Offenlegungs-schriften 198 17 264 (=WO 99/54 326), 198 17 265 (=WO 99/54 312) and 198 17 262 (=WO 99/54 329), the disclosure of which is incorporated herein by reference.

Preferred dihydropyrimidines B(ii) correspond, for example, to the formula

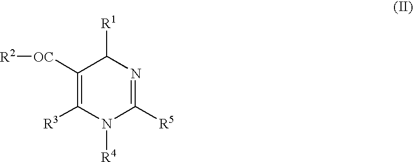

or its isomeric form

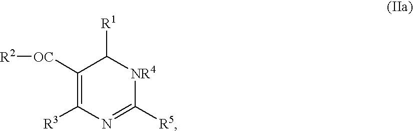

and the salts thereof, in which

R¹ denotes phenyl, furyl, thienyl, triazolyl, pyridyl, cycloalkyl having 3 to 6 carbon atoms or radicals of the formulae

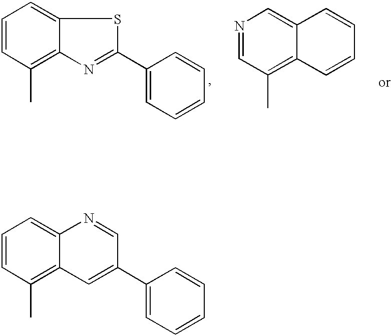

where the ring systems mentioned above are optionally substituted one or more times, identically or differently, by substituents selected from the group of halogen, trifluoromethyl, nitro, cyano, trifluoromethoxy, carboxyl, hydroxyl, C₁-C₆-alkoxy, C₁-C₆-alkoxycarbonyl and C₁-C₆-alkyl, where the alkyl radical in turn may be substituted by aryl having 6 to 10 carbon atoms or halogen,

and the mentioned ring systems are optionally substituted by —S—R⁶, —N⁷R⁸, —CO—NR⁹R¹⁰, —SO₂—CF₃ and —A—CH₂-R¹¹, in which

R⁶ denotes optionally halogen-substituted phenyl,

R⁷ to R¹⁰ denote, independently of one another, hydrogen, phenyl, hydroxy-substituted phenyl, hydroxyl, C₁-C₆-acyl or C₁-C₆-alkyl, where the alkyl radical in turn may be substituted by hydroxyl, C₁-C₆-alkoxycarbonyl, phenyl or hydroxy-substituted phenyl,

A denotes a radical —O—, —S—, —SO— or —SO₂—,

R¹¹ denotes phenyl which is optionally substituted one or more times, identically or differently, by substituents selected from the group of halogen, nitro, trifluoromethyl, C₁-C₆-alkyl and C₁-C₆-alkoxy,

R² denotes a radical of the formulae —XR¹² or —NR¹³R¹⁴,

in which

X denotes a single bond or oxygen,

R¹² denotes hydrogen, straight-chain or branched C₁-C₆-alkoxycarbonyl, a straight-chain, branched or cyclic, saturated or unsaturated C₁-C₈-hydrocarbon radical which optionally contains one or two identical or different hetero chain members from the group of —O—, —CO—, —NH—, —N—(C₁-C₄-alkyl)—, —S— or —SO₂— and which is optionally substituted by halogen, nitro, cyano, hydroxyl, aryl having 6 to 10 carbon atoms, aralkyl having 6 to 10 carbon atoms, heteroaryl or a group of the formula —NR¹⁵ R¹⁶,

in which R¹⁵ and R¹⁶ denote, independently of one another hydrogen, benzyl or C₁-C₆-alkyl,

R¹³ and R¹⁴ denote, independently of one another, hydrogen, C₁-C₆-alkyl or cycloalkyl having 3 to 6 carbon atoms,

R³ denotes hydrogen, amino or a radical of the formula

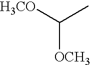

or

formyl, cyano, hydroxy-substituted C₁-C₆-alkylthio, trifluoromethyl or pyridyl or

a straight-chain, branched or cyclic, saturated or unsaturated hydrocarbon radical having up to 8 carbon atoms which is optionally substituted one or more times, identically or differently, by aryloxy having 6 to 10 carbon atoms, azido, halogen, cyano, hydroxyl, carboxyl, C₁-C₆-alkoxycarbonyl, a 5- to 7-membered heterocyclic ring, C₁-C₆-alkylthio or C₁-C₆-alkoxy (where the alkylthio or alkoxy radical may in turn be substituted by azido, amino, hydroxyl) and/or by the group —(CO)ₐ—NR¹⁷ R¹⁸,

in which

a denotes zero or 1,

R¹⁷ and R¹⁸ s denote, independently of one another, hydrogen or aryl having 6 to 10 carbon atoms, aralkyl having 6 to 10 carbon atoms or C₁-C₆-alkyl, each of which is optionally substituted by C₁-C₆-alkoxycarbonyl, amino, hydroxyl, phenyl or benzyl, where phenyl and benzyl are optionally substituted one or more times, identically or differently, by hydroxyl, carboxyl, C₁-C₆-alkyl or C₁-C₆-alkoxy, and/or C₁-C₆-alkyl is optionally substituted by —NH—CO—CH₃ or —NH—CO—CF₃, or

R¹⁷ and R¹⁸ together with the nitrogen atom on which they are located denote a morpholinyl, piperidinyl or pyrrolidinyl ring, or

R³ denotes optionally methoxy-substituted phenyl or

R² and R³ together denote a radical of the formula

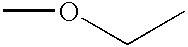

R⁴ denotes hydrogen, C₁-C₄-alkyl, C₂-C₄-alkenyl, benzoyl or acyl having 2 to 6 carbon atoms, preferably hydrogen, methyl, benzoyl or C₂-C₆-acyl, and

R⁵ denotes pyridyl, pyrimidyl or pyrazinyl, each of which may be substituted up to 3 times, identically or differently, by halogen, hydroxyl, cyano, trifluoromethyl, C₁-C₆-alkoxy, C₁-C₆-alkyl, C₁-C₆-alkylthio, carbalkoxy, C₁-C₆-acyloxy, amino, nitro, mono- or di-C₁-C₆-alkylamino.

Very particularly preferred dihydropyrimidines B(ii) are the following compounds:

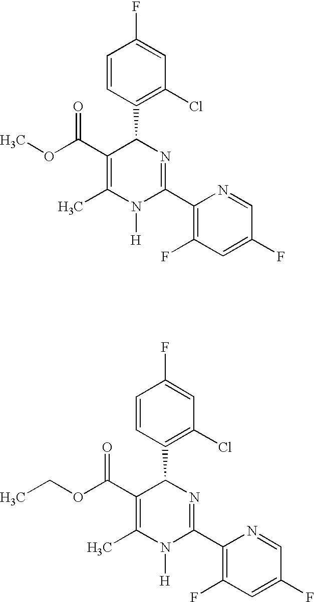

their isomeric forms and their salts.

The compounds II and IIa include the isomers of the formulae (II) and (IIa) and mixtures thereof. If R⁴ is hydrogen, the isomers (II) and (IIa) are present in tautomeric equilibrium:

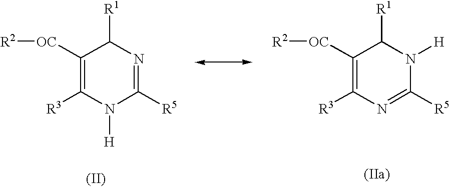

The above dihydropyrimidines II and IIa and various processes for their preparation are disclosed in German Offenlegungsschriften 198 17 264 (=WO 99/54 326) and 198 17 265 (=WO 99/54 312), the disclosure of which is incorporated herein by reference.

Further preferred dihydropyrimidines B(ii) correspond to the formula

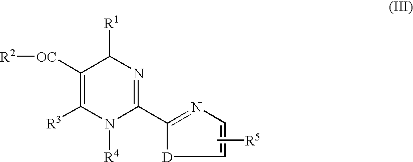

and the isomeric form thereof

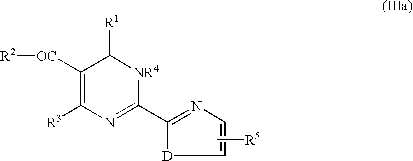

and/or the salts thereof, in which

R¹ denotes phenyl, furyl, thienyl, pyridyl, cycloalkyl having 3 to 6 carbon atoms or a radical of the formulae

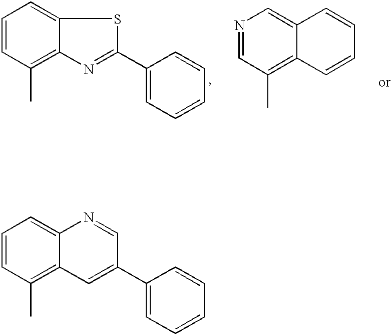

where the ring systems mentioned above are optionally substituted one or more times, identically or differently, by substituents selected from the group of halogen, trifluoromethyl, nitro, cyano, trifluoromethoxy, carboxyl, hydroxyl, C₁-C₆-alkoxy, C₁-C₆-alkoxycarbonyl and C₁-C₆-alkyl, where the alkyl radical in turn may be substituted by aryl having 6 to 10 carbon atoms or halogen,

and/or the mentioned ring systems are optionally substituted by groups of the formulae —S—R⁶, —NR⁷R⁸, —CO—NR⁹R¹⁰,

—SO₂—CF₃ and —A—CH₂-R¹¹,

in which

R⁶ denotes optionally halogen-substituted phenyl,

R⁷ to R¹⁰ denote, independently of one another, hydrogen, phenyl, hydroxy-substituted phenyl, hydroxyl, C₁-C₆-acyl or C₁-C₆-alkyl, where the alkyl radical in turn may be substituted by hydroxyl, C₁-C₆-alkoxycarbonyl, phenyl or hydroxy-substituted phenyl,

A denotes a radical —O—, —S—, —SO— or —SO₂—,

R¹¹ denotes phenyl which is optionally substituted one or more times, identically or differently, by substituents selected from the group of halogen, nitro, trifluoromethyl, C₁-C₆-alkyl and C₁-C₆-alkoxy,

R² denotes a radical of the formulae —OR¹² or —NR¹³R¹⁴,

in which

R¹² denotes hydrogen, C₁-C₆-alkoxycarbonyl or a straight-chain, branched or cyclic, saturated or unsaturated C₁-C₈-hydrocarbon radical which optionally contains one or two identical or different hetero chain members from the group of —O—, —CO—, —NH—, —N—(C₁-C₄-alkyl)—, —S— and —SO₂— and which is optionally substituted by halogen, nitro, cyano, hydroxyl, aryl having 6 to 10 carbon atoms or aralkyl having 6 to 10 carbon atoms, heteroaryl or a group of the formula —NR¹⁵R¹⁶,

in which R¹⁵ and R¹⁶ denote, independently of one another, hydrogen, benzyl or C₁-C₆-alkyl,

R¹³ and R¹⁴ denote, independently of one another, hydrogen, C₁-C₆-alkyl or cycloalkyl having 3 to 6 carbon atoms,

R³ denotes hydrogen, amino or a radical of the formula

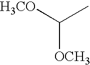

or formyl, cyano, hydroxy-substituted C₁-C₄-alkylthio, trifluoromethyl or a straight-chain, branched or cyclic, saturated or unsaturated hydrocarbon radical having up to 8 carbon atoms, which is optionally substituted one or more times, identically or differently, by aryloxy having 6 to 10 carbon atoms, azido, cyano, hydroxyl, carboxyl, C₁-C₆-alkoxycarbonyl, a 5- to 7-membered heterocyclic ring, C₁-C₆-alkylthio or C₁-C₆-alkoxy (where the alkylthio or alkoxy radical in turn can be substituted by azido, amino or hydroxyl) and/or by the group —(CO)ₐ—NR¹⁷R¹⁸,

in which a denotes zero or 1,

R¹⁷ and R¹⁸ denote, independently of one another, hydrogen or aryl, aralkyl having 6 to 10 carbon atoms or C₁-C₆-alkyl, which are optionally substituted by C₁-C₆-alkoxycarbonyl, amino, hydroxyl, phenyl or benzyl, where phenyl and benzyl are optionally substituted one or more times, identically or differently, by hydroxyl, carboxyl, C₁-C₆-alkyl or C₁-C₆-alkoxy, and/or C₁-C₆-alkyl is optionally substituted by —NH—CO—CH₃ or —NH—CO—CF₃, or

R¹⁷ and R¹⁸ together with the nitrogen atom on which they are located denote a morpholinyl, piperidinyl or pyrrolidinyl ring,

D denotes an oyxgen or sulphur atom and

R⁵ denotes hydrogen, halogen or straight-chain or branched alkyl having up to 6 carbon atoms.

The compounds III and IIIa may exist in stereoisomeric forms which either are related as image and mirror image (enantiomers) or are not related as image and mirror image (diastereomers). The compounds III and IIIa thus encompass both the enantiomers and the diastereomers, and the respective mixtures thereof. The racemic forms can, just like the diastereomers, be separated into the stereoisomerically homogeneous components in a known manner.

The dihydropyrimidines III and IIIa which contain in position 2 an optionally substituted oxazolyl or thiazolyl radical, and various processes for their preparation are disclosed in German Offenlegungsschrift 198 17 262 (=WO 99/54 329), the disclosure which is incorporated herein by reference.

Alkyl per se and the alkyl moieties in mono- and dialkylamino and in mono- and dialkylaminocarbonyl represent within the framework of the dihydropyrimidine definition a linear or branched alkyl radical having 1 to 8, preferably 1 to 6, carbon atoms, such as, for example, methyl, ethyl, propyl, isopropyl, tert-butyl, n-pentyl, n-hexyl, 2-ethylhexyl or n-octyl. Alkenyl represents within the frameowrk of the dihydropyrimidine definition a straight-chain or branched alkenyl radical having 2 to 6, preferably 3 to 5, carbon atoms, such as, for example, ethenyl, propenyl, isopropenyl, tert-butenyl, n-pentenyl and n-hexenyl.

Cycloalkyl having 3 to 6 carbon atoms represents within the framework of the dihydropyrimidine definition cyclopropyl, cyclopentyl, cyclobutyl, cyclohexyl, preferably cyclopentyl and cyclohexyl.

Acyl represents within the framework of the dihydropyrimidine definition a straight-chain or branched acyl radical having 1 to 6, preferably 1 to 4, carbon atoms such as, for example, acetyl and propionyl.

Alkoxy represents within the framework of the dihydropyrimidine definition a straight-chain or branched alkoxyl radical having 1 to 6, preferably 1 to 4, carbon atoms such as, for example, methoxy, ethoxy, propoxy, isopropoxy, tert-butoxy, n-pentoxy and n-hexoxy.

Alkylthio represents within the framework of the dihydropyrimidine definition a straight-chain or branched alkylthio radical having 1 to 6, preferably 1 to 4, carbon atoms such as, for example, methylthio, ethylthio and propylthio.

Alkoxycarbonyl represents within the framework of the dihydropyrimidine definition a straight-chain or branched alkoxylcarbonyl radical having 1 to 6, preferably 1 to 4, carbon atoms such as, for example, methoxycarbonyl, ethoxycarbonyl, propoxycarbonyl, isopropoxycarbonyl, tert-butoxycarbonyl, n-pentoxycarbonyl and n-hexoxycarbonyl.

Aralkyl represents within the framework of the dihydropyrimidine definition aralkyl having, preferably, 6 to 10, in particular, 6, carbon atoms in the aryl moiety (preferably phenyl or naphthyl, in particular phenyl) and preferably 1 to 4, in particular 1 or 2, carbon atoms in the alkyl moiety, where the alkyl moiety can be linear or branched. Preferred aralkyl radicals are benzyl and phenethyl.

Aryl represents within the framework of the dihydropyrimidine definition an aromatic radical having 6 to 10 carbon atoms, preferably phenyl and naphthyl.

Heteroaryl represents within the framework of the dihydropyrimidine definition 5- to 7-membered rings with, preferably, 1 to 3, in particular 1 or 2, identical or different heteroatoms from the series oxygen, sulphur and nitrogen. Preferred examples comprise furyl, thienyl, pyrazolyl, imidazolyl, 1,2,3- and 1,2,4-triazolyl, oxazolyl, isoxazolyl, thiazolyl, isothiazolyl, 1,2,3-, 1,3,4-, 1,2,4- and 1,2,5-oxadiazolyl, pyrrolyl, pyridyl, pyrimidinyl, pyrazinyl, 1,3,5-, 1,2,4- and 1,2,3-triazinyl, 1,2,4-, 1,3,2-, 1,3,6-and 1,2,6-oxazinyl, in particular pyridyl and pyrimidyl.

Halogen represents within the framework of the dihydropyrimidine definition fluorine, chlorine, bromine or iodine. The preferred halogenated alkyl is trifluoromethyl.

The compounds II or IIIa and III or IIIa may also be in the form of salts.

Physiologically acceptable salts are preferred for the purposes of the invention. Physiologically acceptable salts may be salts of the compounds II or IIa and III or IIIa with inorganic or organic acids. Preference is given to salts of inorganic acids such as, for example, hydrochloric acid, hydrobromic acid, phosphoric acid or sulphuric acid, or salts of organic carboxylic or sulphonic acids such as, for example, acetic acid, maleic acid, fumaric acid, malic acid, citric acid, tartaric acid, lactic acid, benzoic acid, or methanesulphonic acid, ethanesulphonic acid, phenylsulphonic acid, toluenesulphonic acid or napthalenedisulphonic acid.

Physiologically acceptable salts may likewise be metal or ammonium salts of the compounds II or IIa and III or IIIa. Particularly preferred examples are sodium, potassium, magnesium or calcium salts, and ammonium salts derived from ammonia or organic amines such as, for example, ethylamine, di- or triethylamine, di- or triethanolamine, dicyclohexylamine, dimethylaminoethanol, arginine, lysine, ethylenediamine or 2-phenylethylamine.

Preferred isoxazoles B(iii) comprise, for example, compounds of the formula

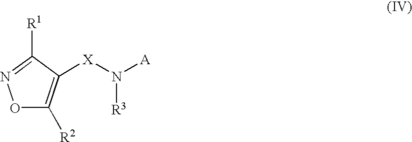

in which

R¹ and R² denote, independently of one another, alkyl which is optionally substituted by one or more halogen atoms,

X denotes a divalent radical from the series C═Y, —N(R⁴)—C(═Y)—, CH₂,

R³ and R⁴ denote, independently of one another, hydrogen or alkyl,

Y denotes an oxygen or sulphur atom and

A denotes aryl or hetaryl which are optionally substituted by 1 to 3 radicals selected, independently of one another, from the series halogen, alkyl, alkoxy, alkylthio, alkoxycarbonyl, aminocarbonylamino, mono- and dialkylamino, cyano, amino, mono- and dialkylaminocarbonyl.

Particularly preferred compounds of the formula (IV) are those in which

R¹ and R² denote, independently of one another, optionally halogen-substituted C₁-C₈-alkyl,

X denotes a divalent radical from the series C═Y and CH₂,

R³ and R⁴ denote, independently of one another, hydrogen or optionally halogen-substituted C₁-C₆-alkyl,

Y denotes an oxygen or sulphur atom and

A denotes phenyl, pyridyl or pyrimidyl, which are optionally substituted by 1 to 3 radicals from the series halogen, C₁-C₆-alkyl, C₁-C₆-alkoxy, C₁-C₆-alkylthio, C₁-C₆-alkoxycarbonyl, carbamoyl, mono-C₁-C₆-alkylaminocarbonyl, di-C₁-C₆-alkylaminocarbonyl, cyano.

Especially preferred compounds of the formula (IV) are those in which

R¹ and R² denote, independently of one another, C₁-C₆-alkyl or trifluoromethyl,

X denotes C═Y,

R³ and R⁴ denote, independently of one another, hydrogen or C₁-C₆-alkyl, preferably hydrogen or methyl,

Y denotes an oxygen or sulphur atom and

A denotes phenyl or pyridyl which are substituted once to three times, preferably 3,4- or 3,5-disubstituted, the substituents of which are selected, independently of one another from the series alkyl, halogen, CF₃, in particular 3-methyl-4-fluoro- and 3-chloro-4-fluorophenyl.

The isoxazoles B(iii) can be prepared from the corresponding acid chloride 2 by reaction with an amine HNAR³:

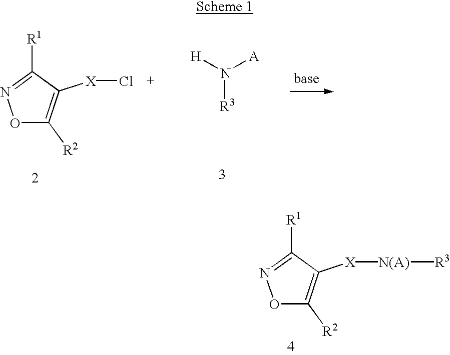

The heterocyclic building block 2 can be synthesized, for example, in analogy to G. Storck, J. E. McMurry, J. Am. Chem. Soc. 1967, 89, 5461 as shown in the following scheme:

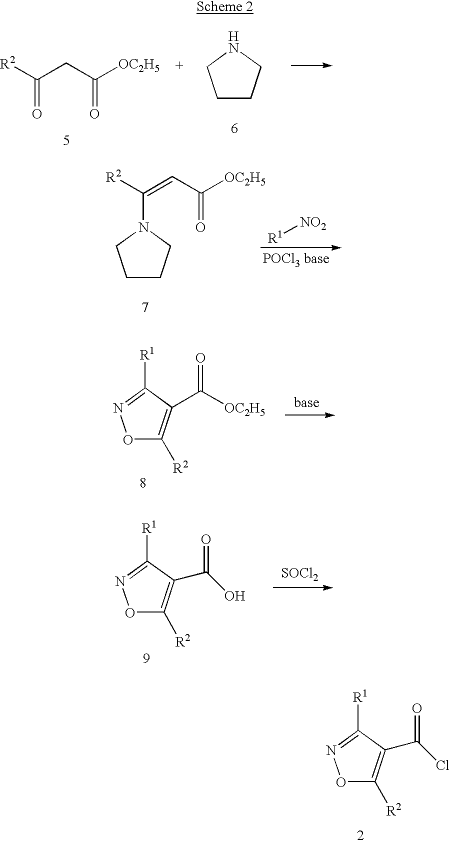

For this purpose, for example, the keto ester 5 is converted with pyrrolidine 6 under water-abstracting conditions into the enamino ester 7 which reacts with an aliphatic nitro compound in the presence of a base, such as, for example, triethylamine and of a water-abstracting agent such as phenyl isocyanate or phosphorus oxychloride to give the isoxazole 8. The ethyl ester can then be cleaved, for example with aqueous sodium hydroxide solution, and the resulting acid 9 can be converted into the acid chloride, for example by treatment with thionyl chloride.

It is possible to use as amine component 3 commercially available anilines or heterocyclic amines.

Bases which can be employed for the reactions in schemes 1 and 2 are in general sodium or lithium bistrimethylsilylamide; alkali metal hydroxides such as sodium hydroxide, lithium hydroxide or potassium hydroxide; sodium bicarbonate, sodium hydride; organic tri-(C₁-C₆)-alkylamines such as triethylamine or diisopropylethylamine; heterocycles such as 1,4-diazabicyclo[5,4,0]undec-7-ene (DBU), pyridin, diaminopyridine, methylpiperidine or N-methylmorpholine.

Preferred bases for the reactions in scheme 1 comprise organic amines such as triethylamine, diisopropylethylamine or N-methylmorpholine, each of which may also be carrier-bound, such as, for example, morpholinomethyl-polystyrene.

Preferred bases for the reactions in scheme 2 comprise lithium hydroxide, pyridine, diisopropylethylamine and triethylamine.

The thioamides (formula I with Y═S) can be synthesized by treating the amides 4 with Lawesson's reagent (=2,4-bis(4-methoxyphenyl)-1,3,2,4-dithiaphosphetane 2,4-disulphide; compare R. Shabana et al., Tetrahedron 1980 (36), 3047-3051); the reaction can take place in toluene at elevated temperature.

The ureas [X═—N(R⁴)—C(═Y)—] can be synthesized by employing, for example, 3-amino-2,5-dimethylisoxazole as starting material (A. Pascual, Helv. Chim. Acta 1989 (72), 556-569) which, after conversion into the carbamoyl chloride, is reacted with amines HNAR in an analogous manner.

The amines (X═CH₂) can be obtained from the corresponding carboxamides described in scheme 1 by reduction, for example with borane/dimethyl sulphide complex (J. March, Advanced Organic Chemistry, 4th edition, New York 1992, p. 1212).

The reactions in schemes 1 and 2 can be carried out in an inert organic solvent. These comprise saturated linear, branched and cyclic hydrocarbons such as hexane, cyclohexane or petroleum fractions, alcohols such as methanol, ethanol or isopropanol, ethers, such as diethyl ether, 1,4-dioxane or tetrahydrofuran, halogenohydrocarbons such as dichloromethane, chloroform, tetrachloromethane, 1,2-dichloroethane, trichloroethane or tetrachloroethane, aromatic hydrocarbons such as benzene, toluene or xylene, dipolar aprotic solvents such as nitromethane, dimethylformamide or acetonitrile, or mixtures thereof. Dichloromethane, chloroform, 1,2-dichloroethane, toluene, ethanol and dimethylformamide are particularly preferred.

Preferred solvents for the reactions in scheme 1 comprise chlorinated hydrocarbons such as dichloromethane, chloroform, 1,2-dichloroethane and ethers such as tetrahydrofuran. The reactions in scheme 2 are preferably carried out in aromatic hydrocarbons such as toluene, chlorinated hydrocarbons such as dichloromethane, chloroform, 1,2-dichlorethane, ethers such as tetrahydrofuran or alkanols such as ethanol.

The reactions in schemes 1 and 2 are generally carried out in a temperature range from 0 to 150, preferably from 0 to 90,° C. The reactions can be carried out under atmospheric, elevated or reduced pressure (for example 0.5 to 5 bar); atmospheric pressure is generally employed.

The invention further relates to combinations of

A) at least one chromanone and/or chromanol,

B) (i) an HBV polymerase inhibitor and/or (ii) a dihydropyrimidine and/or (iii) an isoxazole and, where appropriate,

C) at least one immunomodulator.

A particularly preferred embodiment of the invention relates to combinations of A) above chromanones and/or chromanols and B)(i) lamivudine.

Other preferred HBV-antiviral agents B comprise, for example, phenylpropenamides of the formula

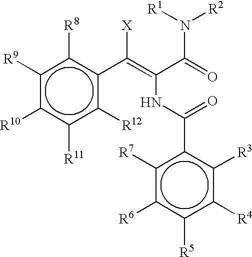

in which

R¹ and R² denote, independently of one another, C₁-C₄-alkyl or form, together with the nitrogen atom on which they are located, a ring having 5 to 6 ring atoms which comprise carbon and/or oxygen,

R³-R¹² denote, independently of one another, hydrogen, halogen, C₁-C₄-alkyl, optionally substituted C₁-C₄-alkoxy, nitro, cyano or trifluoromethyl,

R¹³ denotes hydrogen, C₁-C₄-alkyl, C₁-C₇-acyl or aralkyl and X denotes halogen or optionally substituted C₁-C₄-alkyl,

and the salts thereof.

These phenylpropenamides and processes for their preparation are disclosed in WO 98/33501, the disclosure of which is incorporated herein by reference. AT-61 is a compound of the above formula in which X denotes chlorine, A denotes 1-piperidinyl and Y and Z each denotes phenyl.

Preferred immunomodulators C) comprise, for example, all interferons such as α-, β- and γ-interferons, in particular also α-2a- and α-2b-interferons, interleukins such as interleukin-2, polypeptides such as thymosin α-1 and thymoctonan, imidazoquinoline derivatives such as ®Levamisole, immunoglobulins and therapeutic vaccines.

Another preferred embodiment of the invention relates to combinations of A) at least one chromanone, B(i) lamivudine, B(ii) at least one dihydropyrimidine, B(iii) at least one isoxazole and, where appropriate, C) interferon.

The present invention includes pharmaceutical preparations which, besides non-toxic, inert pharmaceutically suitable carriers, contain one or more compounds (I) or one or more combinations according to the invention or which consist of a combination according to the invention, and to processes for producing these preparations.

The ratio of amounts of components A, B and, where appropriate, C in the compositions according to the invention may vary within wide limits; it is preferably 5 to 1000 mg of A/5 to 500 mg of B, in particular 10 to 500 mg of A/20 to 400 mg of B and, in addition, 5 to 1000 mg of A/5 to 500 mg of B and/or 1 to 10 million I.U. (international units) of C.

The component C which is present where appropriate can preferably be used in amounts of, in particular, 2 to 7 million I.U., about three times a week for a period of up to one year.

The compounds (I) and the combinations according to the invention should generally be present in the abovementioned pharmaceutical preparations in a concentration of about 0.1 to 99.5, preferably about 0.5 to 95,% by weight of the complete mixture.

The abovementioned pharmaceutical preparations may, besides the compounds (I) or besides the combinations according to the invention, also contain other pharmaceutical active substances.

-mentioned pharmaceutical preparations can be produced by known methods, for example by mixing the active substance or active substances with the carrier(s).

The active substances may act systemically and/or locally. For this purpose, they can be administered in a suitable way, such as, for example, by the oral, parenteral, pulmonary, nasal, sublingual, lingual, buccal, rectal, transdermal, conjunctival or otic route or as implant. The active substances can be administered in administration forms suitable for these administration routes.

Suitable for oral administration are administration forms which deliver the active substances rapidly and/or in a modified manner, such as, for example, tablets without or with (for example enteric) coating, capsules, coated tablets, granules, pellets, powder, emulsions or suspensions and solutions.

Parenteral administration can take place with avoidance of an absorption step (intravenous, intraarterial, intracardiac, intraspinal or intralumbar) or with inclusion of an absorption (intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates and sterile powders.

Suitable for the other routes of administration are, for example, pharmaceutical forms for inhalation (inter alia powder inhalers, nebulizers), nasal drops/solutions, sprays; tablets or capsules for lingual, sublingual or buccal administration, suppositories, preparations for the ears and eyes, vaginal capsules, aqueous suspensions (lotions, shaking mixtures), lipophilic suspensions, ointments, creams, milk, pastes, dusting powders or implants.

The active substances can be converted in a manner known per se into the stated administration forms. This takes place with use of inert, non-toxic, pharmaceutically suitable excipients. These include, inter alia, carriers (for example microcrystalline cellulose), solvents (for example liquid polyethylene glycols), emulsifiers (for example sodium dodecyl sulphate), dispersants (for example polyvinylpyrrolidone), synthetic and natural biopolymers (for example albumin), stabilizers (for example antioxidants such as ascorbic acid), colourings (for example inorganic pigments such as iron oxides) or masking flavours and/or odours.

It has generally proved to be advantageous both in human and in veterinary medicine to administer the active substances in total amounts of about 0.5 to about 500, preferably 1 to 100, mg/kg of body weight every 24 hours, where appropriate in the form of a plurality of single doses, to achieve the desired results. A single dose contains the active substance or the active substances preferably in amounts of about 1 to about 80, in particular 1 to 30, mg/kg of body weight. However, it may be necessary to deviate from the dosages mentioned, in particular depending on the species and body weight of the subject to be treated, the nature and severity of the disorder, the type of preparation and mode of administration of the medicament, and the time or interval within which administration takes place.

The areas of indication of the compounds and combinations according to the invention comprise:

1. the treatment of acute and chronic viral infections which may lead to infectious hepatitis, preferably the treatment of acute and chronic hepatitis B viral infections;

2. the treatment of acute and chronic HBV infections where there is coinfection with the hepatitis delta virus;

3. the treatment of acute and chronic HBV infections when there is coinfection with other viruses such as, for example, HIV or HCV, and

4. prophylactic/therapeutic treatment of transplant cases, such as, for example, liver transplants.

The invention therefore further relates to the compounds (I) and combinations defined above for controlling diseases.

The invention further relates to medicaments containing (i) one or more of the compounds (I) defined above or one or more of the combinations defined above and (ii) at least one other pharmaceutical active substance and/or at least one pharmaceutical excipient.

The invention further relates to the use of the combinations defined above for producing medicaments for the treatment and prophylaxis of the diseases described above, preferably of viral diseases, in particular of hepatitis B.

The percentage data in the following examples are based in each case on weight; parts are parts by weight. Mixing ratios of solvent mixtures are based on volume.

### EXAMPLES

A. Chromanones/Chromanols

List of Mobile Phases/Conditions Used for the Chromatography:

I Dichlormethane/methanol

II Dichlormethane

III Ethyl acetate

IV Petroleum ether/ethyl acetate

V Cyclohexane/ethyl acetate

VI Dichlormethane/cyclohexane

VII Cyclohexane

VIII Acetonitrile/water/trifluoroacetic acid

IX Acetonitrile

X Water

XI Kromasil C18 column 125*2 mm, gradient acetonitrile +0.01 molar H₃PO₄, 0-1 min 10% IX, 1-9 min gradient to 90% IX, 9-13 min 90% IX; UV detection 210 nm, flow rate 0.5 ml/min

XII Symmetry C18 2.1*150 mm, gradient acetonitrile +0.6 g of 30% strength HCl, 0 min 10% IX, 0.01-4 min gradient to 90% IX, 4-9 min 90% IX, UW detection 210 nm, flow rate 0.6 ml/min

XIII Kromasil C18 column 125*2 mm, gradient acetonitrile +0.1% HClO₄, 0-0.5 min 2% IX, 0.51-4.5 min gradient to 90% IX, 4.5-6.5 min 90% IX; UV detection 210 nm, flow rate 0.75 ml/min

XIV Kromasil 100 C18 column 7 μm 250*20 mm, gradient acetonitrile +0.2% trifluoroacetic acid, 0-0.01 min 5% IX, 0.01-10 min gradient to 95% IX, 10-15 min 95% IX; UV detection 210 nm, flow rate 25 ml/min

Examples 1 to 43 are starting compounds; the other examples relate to chromanone and chromanol final products.

### Example 1

### 3-Bromphenyl Acetate

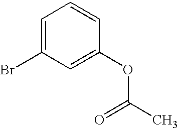

A solution of 49.83 g (288 mnmol) of 3-bromophenol in 94.09 g (921.67 mmol, 86.96 ml) of acetic anhydride is stirred in the presence of 1 ml of concentrated sulphuric acid until the exothermic reaction subsides. This is followed by hydrolysis with 2% strength ice-cold hydrochloric acid, removal of the organic phase, extraction of the aqueous phase three times with diethyl ether, washing of the combined organic phases twice with 2 N sodium carbonate solution and washing once with water. Drying over sodium sulphate and removal of the solvent by distillation result in 43.36 g (73%) of the product in the form of a colourless oil.

¹H-NMR (300 MHz, DMSO-D₆, δ/ppm):

2.25 (s, 3H), 7.15 (dd, 1H), 7.3-7.5 (m, 3H).

MS (EI POS): 214 [M]⁺.

### Example 2

### 3,4-Difluorophenyl Acetate

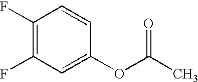

The title compound is obtained on reaction of 3,4-difluorophenol in analogy to

### Example 1.

Yield: quantitative

¹H-NMR (400 MHz, DMSO-D₆, δ/ppm):

2.30 (s, 3H), 7.05 (m, 1H), 7.40 (m, 1H), 7.50 (dd, 1H).

MS (DCI): 173 [M+H]⁺

R_{f}: 0.76 (I, 10:1).

### Example 3

### 4-Bromo-2-hydroxyacetophenone

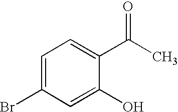

122.77 g (920.74 mmol) of aluminium chloride are added in portions through a solids metering device to 99.00 g (460.37 mmol) of the compound from Example 1. This is followed by heating at 140° C. for 4 h and then hydrolysis with ice-water. The brown solid is filtered off with suction and dissolved in dichloromethane, and the solution is washed three times with water. After removal of the solvent by distillation, the resulting crystals are recrystallized from n-pentane.

Yield: 63 g (63.63%).

¹H-NMR (300 MHz, DMSO-D₆): 2.6 (s, 3H) ppm, 7.18 (dd, 1H) ppm, 7.2 (dd, 1H) ppm, 7.8 (dd, 1H) ppm, 11.99 (br.s, 1H) ppm.

MS (EI POS): 214 [M]⁺.

### Example 4

### 4,5-Difluoro-2-hydroxyacetophenone

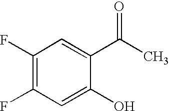

The title compound is obtained by reacting the compound from Example 2 in analogy to Example 3.

| | |
|---|---|
| Yield: | 93% |
| Melting point: | 50-52° C. |
| MS (DCI): | 173 [M + H]⁺ |
| R_{f}: | 0.16 (IV, 20:1). |

### Example 5

### 3-(3-Bromophenoxy)propionic Acid

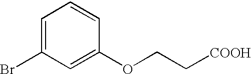

34.60 g (200 mmol) of 3-bromophenol are added to a solution of 8.00 g of sodium hydroxide (200 mmol) in 200 ml of water. After heating to boiling, 30.60 g (200 mmol) of 3-bromopropionic acid and 8.00 g (200 mmol) of sodium hydroxide, dissolved in 400 ml of water, are added dropwise over the course of 30 min. This is followed by stirring under reflux for 2 h, cooling to 0° C. and adjusting to pH 1 with concentrated hydrochloric acid. The precipitated product is filtered off with suction, washed with water and n-pentane and dried. 12.20 g (24.89%) of colourless crystals are obtained.

¹H-NMR (200 MHz, DMSO-D₆, δ/ppm): 2.65 (t, 2H), 4.15 (t, 2H), 6.95 (dd, 1H), 7.10-7.20 (m, 2H), 7.20-7.30 (m, 1H), 12.40 (br, s, 1H).

MS (EI POS): 244 [M⁺].

### Example 6

### 7-Bromospiro[2H-1-benzopyran-2,1′cyclobutan]-4(3H)-one

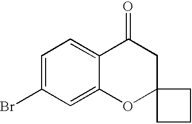

50.00 g (232.51 mmol) of the compound from Example 3 are introduced into 400 ml of toluene. Then 24.45 g (348.76 mmol) of cyclobutanone and 4.96 g (5.82 ml, 69.75 mmol) of pyrrolidine are added. The mixture is stirred at room temperature for 2 hours and heated to boiling with a water trap for 1.5 hours. For workup, 200 ml of 1 N hydrochloric acid are added and the organic phase is separated off, washed with saturated aqueous sodium bicarbonate solution and water and dried over magnesium sulphate. After removal of the solvent by distillation, the product is purified by column chromatography on silica gel (VI 1:2). Subsequent crystallization from n-pentane affords 39.20 g (54.6%) of the title compound.

¹H-NMR (300 MHz, DMSO-D₆, δ/ppm): 1.68-1.92 (m, 2H), 2.05-2.30 (m, 4H), 3.00 (s, 2H), 7.25 (dd, 1H), 7.35 (d, 1H), 7.65 (d, 1H).

MS (DCI/NH₃): 284 [M+NH₄⁺].

### Example 7

The following compound was obtained in analogy to Example 6:

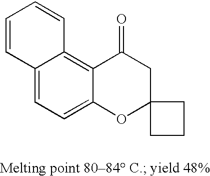

### Example 8

### 7-Bromospiro[2H-1-benzopyran-2,1′cyclopentan]-4(3H)-one

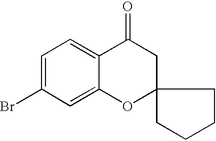

13.00 g (60.45 mmol) of the compound from Example 3 are introduced into 60 ml of toluene. Then 6.61 g (6.95 ml, 78.59 mmol) of cyclopentanone and 1.29 g (1.51 ml, 18.14 mmol) of pyrrolidine are added. The mixture is stirred at room temperature for one hour and then boiled with a water trap for 3 hours. For workup, 20 ml of 1 N hydrochloric acid are added and the organic phase is separated off and washed with 2 N aqueous sodium carbonate solution and water. After drying over sodium sulphate and removal of the solvent by distillation, 13.5 g (79.43%) of yellow crystals are obtained by column chromatography on silica gel (V 20:1).

¹H-NMR (400 MHz, CD₂Cl₂, δ/ppm):

1.60-1.70 (m, 2H), 1.70-1.80 (m, 2H), 1.80-1.92 (m, 2H), 2.00-2.11 (m, 2H), 2.80 (s, 2H), 7.11 (dd, 1H), 7.13 (dd, 1H), 7.69 (d, 1H).

MS (ESI; acetonitrile/water 70:30+0.1% acetic acid): 281 [M+H]⁺, 303 [M+Na]⁺.

The following compounds were prepared in analogy to Example 8:

### Example 9

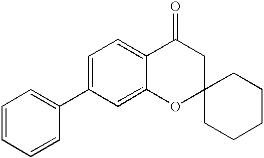

Yield: 60%.

¹H-NMR (200 MHz, CDCl₃, δ/ppm): 1.25-2.2 (m, 10H), 2.70 (s, 1H), 2.85 (s, 1H), 7.15-7.30 (m, 2H), 7.35-7.50 (m, 3H), 7.60-7.70 (m, 2H), 7.90-8.00 (dd, 1H).

MS (EI POS): 293 [M+H]⁺.

### Example 10

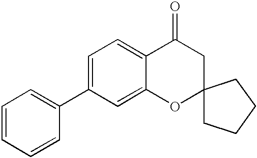

Yield: 24%.

¹H-NMR (200 MHz, CDCl₃, δ/ppm): 1.55-2.00 (m, 6H), 2.05-2.20 (m, 2H), 2.85 (s, 2H), 7.15-7.30 (m, 2H), 7.35-7.55 (m, 3H), 7.60-7.70 (m, 2H), 7.95 (d, 1H).

MS (EI POS): 278 [M]⁺.

### Example 11

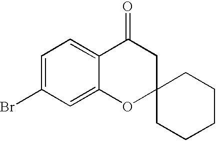

Yield: 95%.

¹H-NMR (400 MHz, DMSO-D₆, δ/ppm):

1.20-2.00 (m, 12H), 2.70 (s, 2H), 7.10 (dd, 1H), 7.20 (d, 1H), 7.70 (d, 1H).

R_{f}: 0.62 (I, 100:1)

MS (DCI): 295 [M+H]⁺.

### Example 12

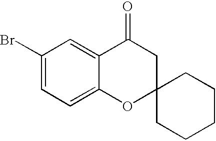

Yield: quantitative.

¹H-NMR (400 MHz, CDCl₃, δ/ppm):

1.25-2.00 (m, 10H), 2.70 (s, 2H), 7.20 (d, 1H), 7.40 (s, 1H), 7.55 (d, 1H).

R_{f}: 0.6 (I, 100:1)

MS (DCI): 295 [M+H]⁺.

### Example 13

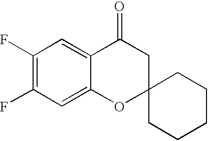

Yield: 68%.

¹H-NMR (400 MHz, CDCl₃, δ/ppm):

1.30-1.75 (m, 8H), 2.00 (m, 2H), 2.70 (s, 2H), 6.80 (dd, 1H), 7.65 (dd, 1H).

R_{f}: 0.58 (II)

MS (DCI): 253 [M+H]⁺.

### Example 14

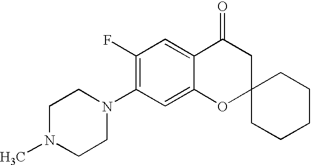

Yield: 75%.

¹H-NMR (400 MHz, CDCl₃, δ/ppm): 1.25-2.00 (m, 10H), 2.35 (s, 3H), 2.55 (m, 4H), 2.65 (s, 2H), 3.25 (m, 4H), 6.40 (d, 1H), 7.40 (d, 1H).

R_{f}: 0.64 (I, 10:1)

MS (DCI): 333 [M+H]⁺.

### Example 15

### (Diastereomer A)

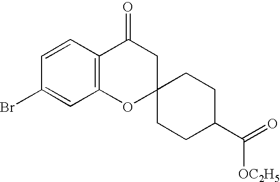

Yield: 89%.

¹H-NMR (300 MHz, DMSO-D₆, δ/ppm): 1.20 (t, 3H), 1.59-1.92 (m, 8H), 2.50-2.62 (m, 1H), 2.85 (s, 2H), 4.08 (q, 2H), 7.23 (dd, 1H), 7.30 (dd, 1H), 7.63 (d, 1H).

MS (DCI/NH₃): 384 [M+NH₄⁺].

### Example 16

### (Diastereomer B)

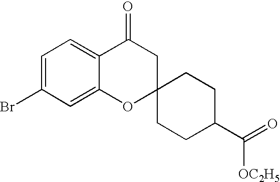

Yield: 55%.

¹H-NMR (300 MHz, 300 K, DMSO-D₆, δ/ppm): 1.18 (t, 3H), 1.45-1.80 (m, 6H), 1.94-2.14 (m, 2H), 2.25-2.41 (m, 1H), 2.77 (s, 2H), 4.07 (q, 2H), 7.22 (dd, 1H), 7.34 (d, 1H), 7.65 (d, 1H).

MS (DCI/NH₃): 384 [M+NH₄⁺].

### Example 17

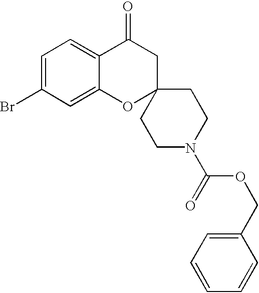

Melting point. 90-97° C.; Yield 75%.

### Example 18

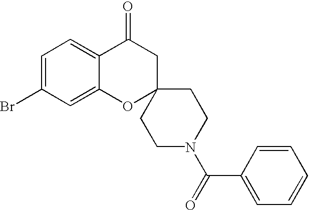

Yield: 97%.

¹H-NMR (300 MHz, 300 K, DMSO-D₆, δ/ppm): 1.60-2.10 (m, 4H), 2.90 (s, 2H), 3.10-3.60 (m, 4H), 7.25 (dd, 1H), 7.37-7.50 (m, 6H), 7.65 (d, 1H).

MS (EI POS): 399 [M]⁺.

### Example 19

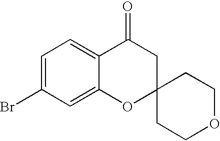

Yield: 35%.

¹H-NMR (300 MHz, 300 K, DMSO-D₆, δ/ppm): 1.60-1.90 (m, 4H), 2.90 (s, 2H), 3.60-3.80 (m, 4H), 7.25 (dd, 1H), 7.38 (dd, 1H), 7.65 (d, 1H).

MS (EI POS): 296 [M]⁺.

### Example 20

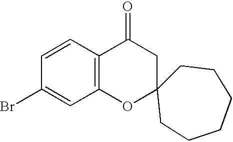

Yield: 38%.

¹H-NMR (300 MHz, DMSO-D₆, δ/ppm): 1.34-1.80 (m, 10H), 1.90-2.05 (m, 2H), 2.82 (s, 2H), 7.20 (dd, 1H), 7.28 (d, 1H), 7.62 (d, 1H).

MS (EI POS): 308 [M+H]⁺.

### Example 21

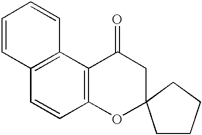

Yield: 51%.

¹H-NMR (200 MHz, 300 K, DMSO-D₆, δ/ppm): 1.60-2.15 (m, 8H), 2.99 (s, 2H), 7.18 (d, 1H), 7.38-7.50 (m, 1H), 7.53-7.70 (m, 1H), 7.90 (d, 1H), 8.10 (d, 1H), 9.32 (d, 1H).

MS (DCI/NH₃): 253 [M+H⁺], 270 [M+NH₄⁺].

### Example 22

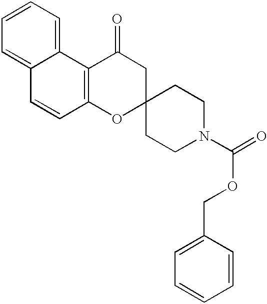

Melting point 43-50° C.; Yield: 51%.

### Example 23

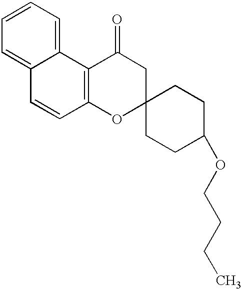

Yield: 25%.

¹H-NMR (300 MHz, 300 K, DMSO-D₆, δ/ppm): 0.90 (t, 3H), 1.20-1.90 (m, 12H), 2.85 (s. 2H), 3.35 (t, 2H), 3.50-3.60 (m, 1H), 7.20 (d, 1H), 7.40-7.45 (m, 1H), 7.60-7.65 (m, 1H), 7.90 (d, 1H), 8.10 (d, 1H), 9.30 (d, 1H).

MS (ESI POS): 339 [M+H]⁺.

### Example 24

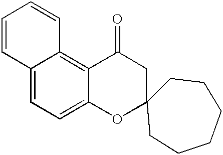

Melting point 81-85,° C.; Yield: 70%.

### Example 25

### 7-Bromo-2,2-dimethyl-2,3-dihydro-4H-chromen-4-one

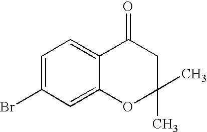

7.02 g (8.89 ml, 120.90 mmol) of acetone and 1.98 g (2.33 ml, 27.90 mmol) of pyrrolidine are added to a solution of 20.00 g (93.00 mmol) of the compound from Example 3 in 100 ml of toluene. The mixture is boiled with a water trap for 20 hours. The solvent is removed by distillation and the product is isolated by chromatography on silica gel (cyclohexane/ethyl acetate 20:1). Recovered precursor is stirred in 100 ml of benzene and 50 ml of acetone in the presence of 5.00 ml of piperidine under reflux for 16 h. After removal of the solvent by distillation, the product is purified by column chromatography on silica gel (V 20:1). Yellow crystals are obtained from the combined oils from ethyl acetate/petroleum ether and are filtered off with suction and washed with diethyl ether.

Yield: 5.95 g (25.08%).

¹H-NMR (200 MHz, CDCl₃, δ/ppm):

1.45 (s, 6H), 2.70 (s, 2H), 7.10 (dd, 1H), 7.25 (s, 1H), 7.70 (d, 1H).

MS (DCI/NH₃): 255 [M+H]⁺, 272 [M+NH₄]⁺.

In analogy to Example 25:

### Example 26

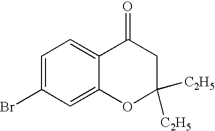

Yield: 43%.

¹H-NMR (300 MHz, 300 K, DMSO-D₆, δ/ppm): 0.80-0.96 (m, 6H), 1.50-1.80 (m, 4H), 2.80 (s, 2H), 7.20 (dd, 1H), 7.27 (d, 1H), 7.62 (dd, 1H).

MS (EI POS): 282 [M]⁺.

### Example 27

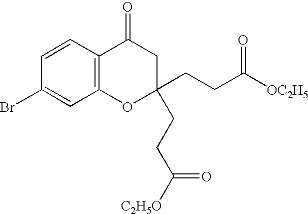

Yield: 43%.

MS (LC-MS): 4,74 (RT/min), 427 [M+H]⁺.

### Example 28

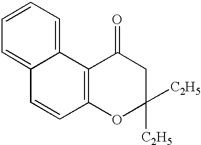

Yield: 29%.

¹H-NMR (300 MHz, 300 K, DMSO-D₆, δ/ppm):

0.90 (t, 6H), 1.65-1.90 (m, 4H), 2.85 (s, 2H), 7.15 (d, 1H), 7.42 (t, 1H), 7.60 (t, 1H), 7.85 (d,1H), 8.10 (d, 1H), 9.30 (d, 1H).

MS (ESI POS): 255 [M+H]⁺.

### Example 29

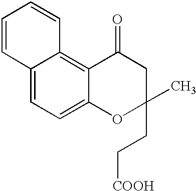

Yield: 7.25%.

MS (DCI/NH₃): 285 [M+H]⁺, 302 [M+NH₄]⁺.

HPLC (XI): RT=8.00.

### Example 30

### 7-Bromo-2,3-dihydro-4H-chromen-4-one

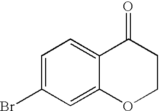

2.45 g (10 mmol) of the compound from Example 5 are added to 3.12 g (15.00 mmol) of phosphorus pentachloride. The mixture is stirred at room temperature for O min, resulting in a clear solution. After addition of 2.67 g (20 mmol) of aluminium chloride, the reaction mixture becomes solid. It is heated at 130° C. for 30 min and, after cooling, added to ice and extracted with ethyl acetate. The organic phase is washed with water and saturated aqueous sodium chloride solution, dried over magnesium sulphate and, after removal of the solvent by distillation, purified by column chromatography on silica gel (V 8:2). Recrystallization from cyclohexane/pentane affords 0.78 g (34%) of colourless crystals.

¹H-NMR (200 MHz, CDCl₃, δ/ppm): 2.80 (t, 2H), 4.55 (t, 2H), 7.15 (dd, 1H), 7.25 (s, 1H), 7.71 (t, 1H).

MS (EI POS): 226 [M⁺].

### Example 31

### 7-(3-Pyridyl)spiro[2H-1-benzopyran-2,1′cyclohexanl-4(3H)-one

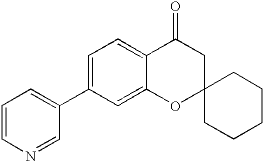

0.85 g (5.76 nmol) of diethyl(3-pyridyl)borane and 0.11 g (0.09 mmol) of tetrakistriphenylphosphinepalladium are added to a solution of 1.00 g (3.39 mmol) of the compound from Example 11 in 20 ml of dioxane. The mixture is heated under reflux for one hour and, after cooling and addition of 2.4 ml of aqueous 2 M sodium carbonate solution, stirred under reflux for a further 15 hours.

All volatile components are removed under reduced pressure. The residue is taken up in dichloromethane/methanol. The solution is dried over sodium sulphate, and the solvent is stripped off. The residue is chromatographed on silica gel (mobile phase: II:I 20:1).

Yield: 555 mg (56%).

¹H-NMR (400 MHz, DMSO-D₆, δ/ppm):

1.20-1.70 (m, 8H), 1.90 (br. d, 2H), 2.80 (s, 2H), 7.40 (m, 2H), 7.50 (dd, 1H), 7.80 (d, 1H), 8.20 (d, 1H), 8.65 (d, 1H), 8.95 (s, 1H).

MS (DCI): 294 [M+H]⁺

R_{f}: 0.29 (1, 50:1).

The following compounds were obtained in analogy to Example 31:

### Example 32

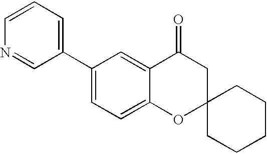

Yield: 51%.

R_{f}:=0.52 (I, 20.1)

¹H-NMR (400 MHz, CDCl₃, δ/ppm):

1.30-1.80 (m, 6H), 2.05 (br. d, 2H), 2.75 (s, 2H), 7.10 (d, 1H), 7.35 (dd, 1H), 7.75 (dd, 1H), 7.85 (dd, 1H), 8.10 (d, 1H), 8.60 (d, 1H), 8.85 (br. s, 1H).

MS (DCI): 294 [M+H]⁺

### Example 33

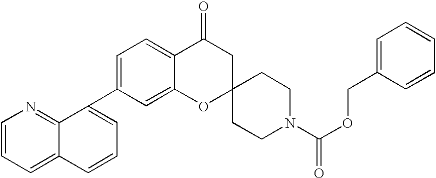

Yield: 72%.

¹H-NMR (300 MHz, 300 K, DMSO-D₆, δ/ppm):

1.60-1.80 (m, 2H), 1.90-2.10 (m, 2H), 2.90 (s, 2H), 3.10-3.40 (m, 2H), 3.80-3.90 (m, 2H), 5.10 (s, 2H), 7.28-7.41 (m, 5H), 7.50-7.78 (m, 4H), 7.79-7.90 (m, 2H), 8.10 (d, 1H), 8.30 (dd, 1H), 8.90-9.00 (m, 1H).

MS (ESI acetonitrile/water 7:3+0.1% acetic acid): 479 [M+H]⁺.

### Example 34

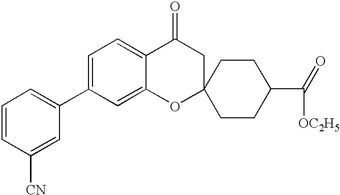

Yield: 28%.

MS (DCI/NH₃): 407 [M+NH₄]⁺

¹H-NMR (300 MHz, 300 K, DMSO-D₆, δ/ppm):

1.20 (t, 3H), 1.55-2.00 (m, 8H), 2.55-2.65 (m, 1H), 2.88 (s, 2H), 4.02-4.15 (m, 2H), 7.40-7.50 (m, 2H), 7.62-7.75 (m, 1H), 7.75-7.85 (m, 1H), 7.85-7.95 (m,1H), 8.05-8.15 (m, 1H), 8.25-8.35 (m, 1H).

### Example 35

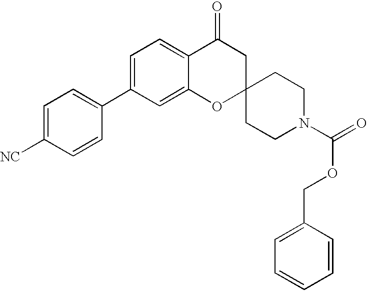

Yield: 87%.

¹H-NMR (300 MHz, 300 K, DMSO-D₆, δ/ppm): 2.60-2.80 (m, 2H), 2.90 (s, 2H), 2.90-3.00 (m, 2H), 3.10-3.40 (m, 2H), 3.75-3.90 (m, 2H), 5.10 (s, 2H), 7.25-7.40 (m, 5H), 7.45 (dd, 1H), 7.48-7.53 (m, 1H), 7.85 (d, 1H), 7.96 (s, 4H).

MS (DCI/NH₃):470 [M+NH₄]⁺.

### Example 36

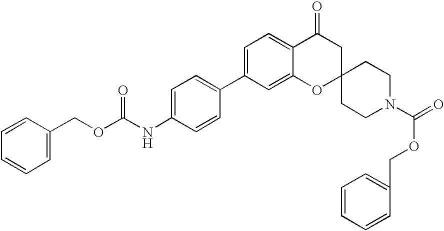

Yield: 63%.

¹H-NMR (400 MHz, 300 K, DMSO-D₆, δ/ppm): 1.60-1.78 (m, 2H), 1.90-2.00 (m, 2H), 2.85 (s, 2H), 3.79-3.88 (m, 2H), 5.10 (s, 2H), 5.20 (s, 2H), 7.29-7.50 (m, 12H), 7.50-7.68 (m, 2H), 7.70 (d, 2H), 7.80 (d, 1H), 10.00 (s, 1H).

MS (ESI POS): 577 [M+H]⁺.

### Example 37

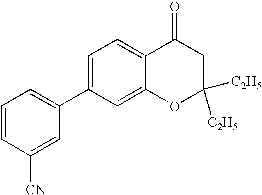

Yield: 96%.

¹H-NMR (400 MHz, 300 K, DMSO-D₆, δ/ppm): 0.89 (t, 6H), 1.64-1.81 (m, 4H), 2.82 (s, 2H), 7.39-7.44 (m, 2H), 7.65-7.71 (m, 1H), 7.80 (d, 1H), 7.88 (d, 1H), 8.09 (d, 1H), 8.24-8.27 (m, 1H).

MS (DCI/NH₃): 323 [M+NH₄]⁺, 306 [M+H]⁺.

### Example 38

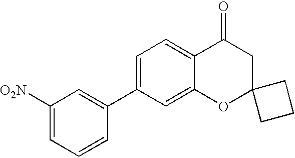

Melting point 108-110° C.; yield: 67%.

### Example 39

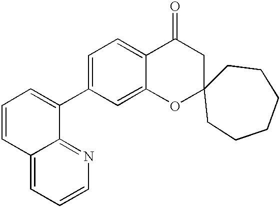

Yield: 50%.

¹H-NMR (300 MHz, 300 K, DMSO-D₆, δ/ppm): 1.38-1.72 (m, 8H), 1.74-1.87 (m, 2H), 1.98-2.10 (m, 2H), 2.85 (s, 2H), 7.25-7.35 (m, 2H), 7.55-7.63 (m, 1H), 7.64-7.82 (m, 3H), 8.05 (d, 1H), 8.46 (d, 1H), 8.90-8.95 (m, 1H).

MS (DCI /NH3)=358 [M+H]⁺.

### Example 40

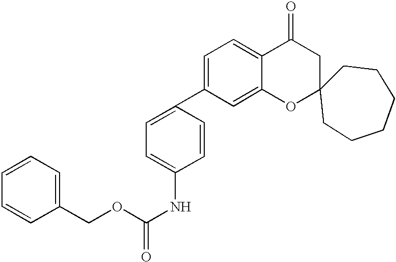

Yield: 58%.

¹H-NMR (300 MHz, 300 K, DMSO-D₆, δ/ppm):

1.30-1.80 (m, 10H), 1.90-2.05 (m, 2H), 2.80 (s, 2H), 5.20 (s, 2H), 7.20-7.50 (m, 7H), 7.58 (d, 2H), 7.66-7.78 (m, 3H), 9.80 (s, 1H).

MS (EI POS): 455 [M]⁺.

### Example 41

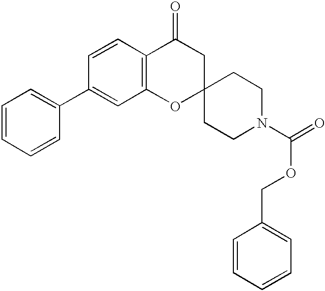

Yield: 52%.

¹H-NMR (300 MHz, 300 K, DMSO-D₆, δ/ppm):

1.50-1.80 (m, 2H), 1.80-2.05 (m, 2H), 2.90 (s, 2H), 3.10-3.40 (m, 2H), 3.70-3.90 (m, 2H), 5.10 (s, 2H), 7.20-7.55 (m, 10H), 7.70-7.90 (m, 3H).

MS (ESI acetonitrile/water 7:3+0.1% acetic acid): 428 [M+H]⁺.

### Example 42

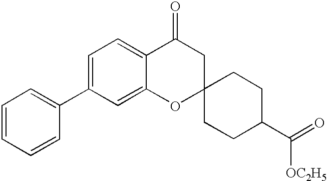

Melting point 108-112° C.; yield: 20%.

### Example 43

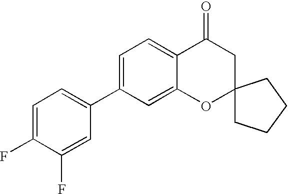

Yield: 88%.

¹H-NMR (200 MHz, 300 K, CDCl₃, δ/ppm): 1.59-2.00 (m, 6H), 2.00-2.21 (m, 2H), 2.85 (s, 2H), 7.05-7.50 (m, 5H), 7.92 (d, 1H).

MS (DCI/NH3): 315 [M+H]⁺.

### Example 44

### 7-Bromo-3-(2-cyanoethyl)spiro[2H-1-benzopyran-2,1′-cyclobutan]-4(3H)-one

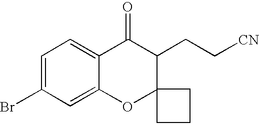

Under argon, 2.00 g (7.49 mmol) of the compound from Example 6 are introduced into 200 ml of tetrahydrofuran, and 0.18 g (7.49 mmol) of sodium hydride is added.

Then 0.60 g (11.23 mmol, 0.74 ml) of acrylonitrile is slowly added dropwise and the reaction mixture is heated to boiling for 2 h. After cooling, 100 ml of water are added and the mixture is extracted with ethyl acetate. The organic phase is separated off and dried over sodium sulphate, and the solvent is removed by distillation. Chromatography on silica gel (1.VII, 2. VI (1:1), 3. V (5:1)) and subsequent recrystallization from ethyl acetate/n-pentane result in 0.60 g (25.03%) of colourless crystals.

¹H-NMR (300 MHz, CDCl₃, δ/ppm):

1.70-1.92 (m, 2H), 1.92-2.12 (m, 2H), 2.12-2.20 (m, 2H), 2.21-2.55 (m, 4H), 2.69 (dd, 1H), 7.10-7.22 (m, 2H), 7.69 (d, 1H).

MS (ESI, acetonitrile/water 7:3+1% acetic acid): 320 [M+H]⁺, 342 [M+Na]⁺.

### Example 45

### 7-Bromo-3,3-bis-(2-cyanoethyl)spiro[2H-1-benzopyran-2,1′-cyclobutan]-4(3H)-one

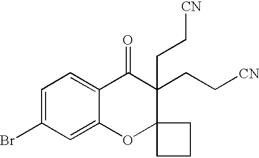

Under argon, 2.50 g (9.36 nunol) of the compound from Example 6 are introduced into 20 ml of dioxane, and 0.45 g (18.72 mmol) of sodium hydride is added. After stirring at room temperature for 30 min, 1.69 g (31.82 nunol) of acrylonitrile are added dropwise and the reaction mixture is heated under reflux for 4 h. The solvent is removed by distillation. Chromatography on silica gel (V (4: 1)) results in 0.34 g (1 1.34%) of the desired compound.

¹H-NMR (300 MHz, DMSO-D₆, δ/ppm): 1.90-2.15 (m, 8H), 2.35-2.45 (m, 1H), 2.52-2.80 (m, 5H), 7.30 (dd, 1H), 7.40 (d, 1H), 7.60 (d, 1H).

MS (DCI/NH₃): 373 [M+H]⁺, 390 [M+NH₄]⁺.

The monoalkylated compounds listed below are obtained in reactions with acrylic acid derivatives in analogy to Example 44:

### Example 46

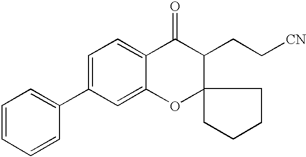

Yield: 9%.

Melting point 75-78° C.

R_{f} 0.52 (I, 100:1)

MS (DCI): 294 [M+H]⁺.

### Example 47

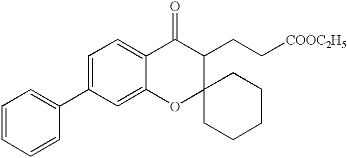

Yield: 31%.

R_{f} 0.48 (1, 100:1)

¹H-NMR (400 MHz, CDCl₃, δ/ppm): 1.25 (t, 3H), 1.40-2.40 (m, 15H), 4.15 (q, 2H), 7.20 (s, 1H), 7.25 (d, 1H), 7.35-7.50 (m, 3H), 7.65 (m, 2H), 7.9 (d, 1H).

MS (DCI): 393 [M+H]⁺.

### Example 48

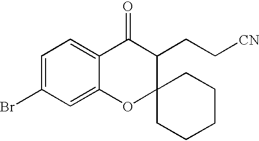

Yield: 12%.

R_{f} 0.57 (1,100:1)

¹H-NMR (400 MHz, CDCl₃, δ/ppm): 1.25-1.40 (m, 3H), 1.55-1.80 (m, 5H), 2.00 (m, 4H), 2.40-2.65 (m, 3H), 7.15 (dd, 1H), 7.20 (d, 1H), 7.70 (d, 1H).

MS (DCI): 348 4+H]⁺

### Example 49

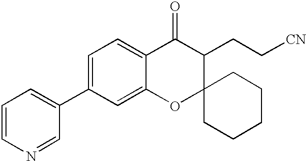

Yield: 22%.

R_{f} 0.5 (1,20:1)

¹H-NMR (400 MHz, CDCl₃, δ/ppm):

1.30 (m, 3H), 1.55-1.75 (m, 5H), 2.05 (m, 4H), 2.40-2.70 (m, 3H), 7.00-7.40 (m, 4H), 7.90 (s, 1H), 7.95 (d, 1H), 8.65 (br. s, 1H), 8.85 (br. s, 1H).

MS (DCI): 347 [M+H]⁺.

### Example 50

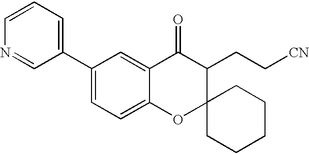

Yield: 2%.

R_{f} 0.51 (1,20:1)

¹H-NMR (400 MHz, CDCl₃, δ/ppm): 1.15-1.30 (m, 3H), 1.40-1.65 (m, 5H), 1.95 (m, 4H), 2.30-2.55 (m, 3H), 7.00 (d, 1H), 7.35 (br. s, 1H), 7.65 (dd, 1H), 7.85 (d, 1H), 7.95 (s, 1H), 8.50 (br. s, 1H), 8.75 (br. s, 1H).

MS (DCI): 347 [M+H]⁺.

### Example 51

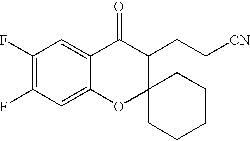

Yield: 24%.

Melting point 80° C.; R_{f} 0.36 (II)

MS (DCI): 306 [M+H]⁺

### Example 52

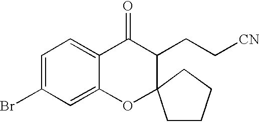

Yield: 19%.

R_{f}: 0.29 (V, 4:1)

¹H-NMR (300 MHz, 300 K, CD₂Cl₂): 1.70-2.30 (m, 10H), 2.45-2.65 (m, 2H), 2.70 (dd, 1H), 7.30-7.35 (m, 2H), 7.80-7.88 (m, 1H).

MS (DCI/NH₃): 351 [M+NH₄]⁺.

### Example 53

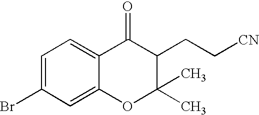

Yield: 6.5%.

¹H-NMR (400 MHz, 300 K, CD₂Cl₂): 1.30 (s, 3H), 1.52 (s, 3H), 1.80-1.90 (m, 1H), 1.91-2.08 (m, 1H), 2.43-2.52 (m, 1H), 2.60-2.69 (m, 1H), 2.70 (dd, 1H), 7.11-7.16 (m, 2H), 7.66 (d, 1H).

MS (DCI/NH₃): 325 [M+NH₄]⁺

### Example 54

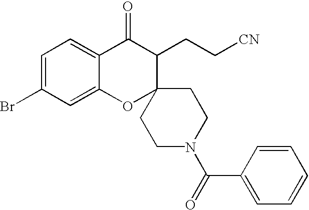

Melting point 72-82° C.; yield: 59%.

### Example 55

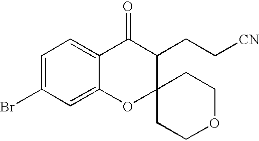

Melting point 107-11 1° C.; yield: 25%.

### Example 56

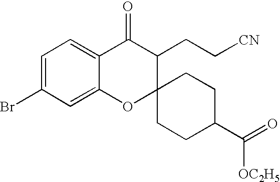

Yield: 24%.

¹H-NMR (400 MHz, 300 K, CD₂Cl₂, δ/ppm):

1.16 (t, 3H), 1.58-1.68 (m, 1H), 1.70-1.90 (m, 9H), 2.25-2.45 (m, 2H), 2.50-2.58 (m, 2H), 4.04 (q, 2H), 7.07 (dd, 1H), 7.12 (d, 1H), 7.58 (d, 1H).

MS (DCI/NH₃): 437 [M+NH₄]⁺.

### Example 57

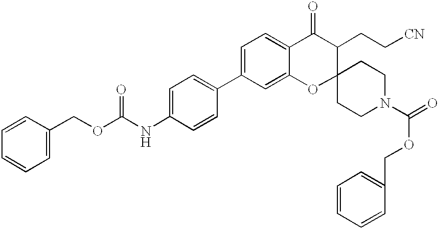

Melting point 89-94° C.; yield: 18%.

### Example 58

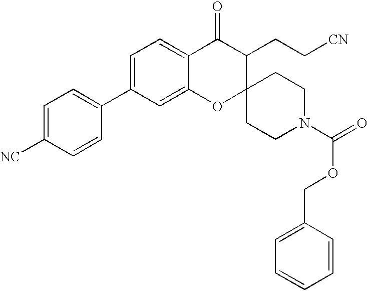

Yield: 5%.

MS (ESI POS)=506 [M+H]⁺.

LC-MS (XII): 5.13 (RT/min).

### Example 59

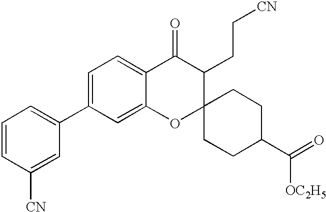

Yield: 39%.

MS (DCI/NH₃): 460 [M+NH₄]⁺.

### Example 60

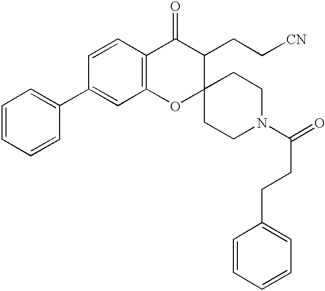

Melting point 64-69° C.; yield: 32%.

### Example 61

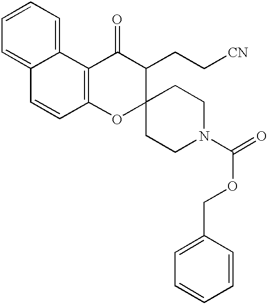

Yield: 48%.

¹H-NMR (400 MHz, DMSO-D₆, δ/ppm):

1.60-1.70 (m, 1H), 1.80-1.90 (m, 2H), 1.90-2.10 (m, 2H), 2.38-2.60 (m, 2H), 2.80 (dd, 1H), 3.10-3.25 (m, 2H), 3.90 (m, 2H), 5.10 (s, 2H), 7.28 (d, 1H), 7.30-7.42 (m, 5H), 7.46 (t, 1H), 7.65 (dt, 1H), 7.92 (d, 1H), 8.18 (d, 1H), 9.22 (d, 1H).

MS (ESI, acetonitrile/water 7:3+1% acetic acid): 455 [M+H]⁺.

### Example 62

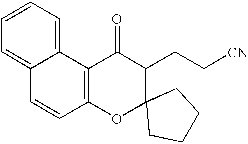

Yield: 26%.

¹H-NMR (300 MHz, DMSO-D₆, δ/ppm):

1.58-2.10 (m, 12H), 2.70 (dd, 1H), 7.18 (d, 1H), 7.46 (dt, 1H), 7.64 (dt, 1H), 7.90 (d, 1H), 8.12 (d, 1H), 9.27 (d, 1H).

MS (EI POS)=305 [M]⁺.

The following dialkylated compounds were obtained in reactions with acrylic acid derivatives in analogy to Example 45:

### Example 63

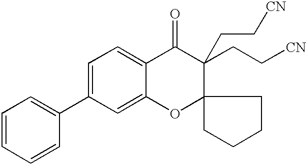

Yield: 15%.

R_{f}: 0.43 (I, 100:1)

¹H-NMR (400 MHz, CDCl₃, δ/ppm): 1.30-2.60 (m, 16H), 7.20 (s, 1H), 7.25 (d, 1H), 7.35-7.50 (m, 3H), 7.65 (m, 2H), 7.9 (d, 1H).

MS (DCI): 385 [M+H]⁺.

### Example 64

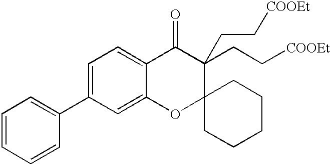

Yield: 10%.

R_{f}: 0.41 (1, 100:1)

¹H-NMR (400 MHz, CDCl₃, δ/ppm): 1.25 (m, 6H), 1.50-2.50 (m, 18H), 4.15 (q, 4H), 7.20 (s, 1H), 7.25 (d, 1H), 7.35-7.50 (m, 3H), 7.60 (m, 2H), 7.85 (dd, 1H).

MS (DCI): 493 [M+H]⁺.

### Example 65

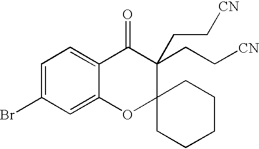

Yield: 14%.

Melting point 145-149° C.; R_{f}: 0.36 (I, 100:1)

MS (DCI): 403 [M+H]⁺.

### Example 66

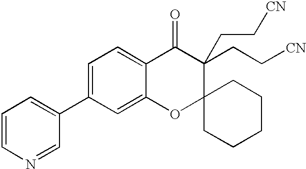

Yield: 38%.

Melting point 190-195° C.; R_{f}: 0.34 (I, 20:1)

MS (DCI): 400 [M+H]⁺.

### Example 67

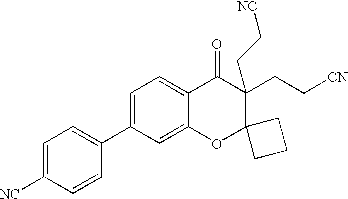

Yield: 61%.

¹H-NMR (200 MHz, 300 K, DMSO-D₆): 1.65-1.88 (m, 1H), 1.90-2.22 (m, 7H), 2.40-2.55 (m, 1H), 2.55-2.90 (m, 5H), 7.45-7.55 (m, 2H), 7.80 (d, 1H), 7.90-8.05 (m,4H).

MS (DCI/NH₃): 413 [M+NH₄]⁺.

### Example 68

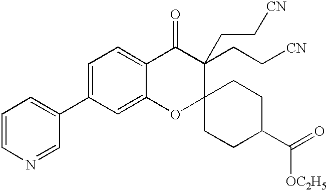

Yield: 7.50%.

¹H-NMR (400 MHz, 300 K, DMSO-D₆): 1.18 (t, 3H), 1.50-2.30 (m, 13H), 2.50-2.70 (m, 3H), 4.05 (q, 2H), 7.45 (s, 1H), 7.46-7.55 (m, 1H), 7.82 (d, 1H), 8.20 (td, 1H), 8.62 (d, 1H), 8.99 (d, 1H).

MS (DCI/NH₃): 472 [M+H]⁺.

### Example 69

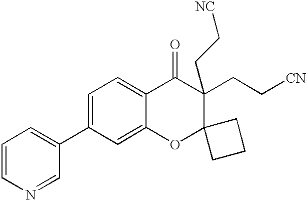

Melting point 148-151° C.; yield: 39%.

### Example 70

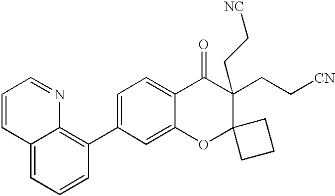

Yield: 44%.

¹H-NMR (400 MHz, 300 K, CD₂Cl₂, δ/ppm):

1.75-1.91 (m, 1H), 2.10-2.30 (m, 7H), 2.30-2.40 (m, 2H), 2.42-2.54 (m, 2H), 2.58-2.72 (m, 2H), 7.25-7.30 (m, 1H), 7.32 (dd, 1H), 7.35-7.42 (m, 1H), 7.50-7.60 (m, 1H), 7.68 (dd, 1H), 7.75 (d, 1H), 7.84 (dd, 1H), 8.18 (dd, 1H), 8.84 (dd, 1H).

MS (ESI acetonitrile/water 7:3+0.1% acetic acid): 422 [M+H]⁺.

### Example 71

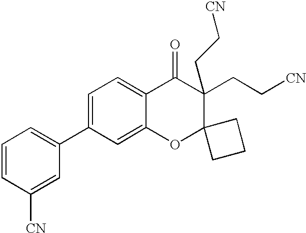

Melting point 187-193° C.; yield: 74%.

### Example 72

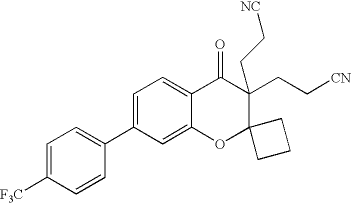

Yield: 90%.

¹H-NMR (400 MHz, 300 K, CD₂Cl₂, δ/ppm):

2.00-2.24 (m, 8H), 2.28-2.38 (m, 2H), 2.41-2.51 (m, 2H), 2.58-2.70 (m, 2H), 7.20 (d, 1H), 7.24 (dd, 1H), 7.79 (d, 1H).

MS (ESI, acetonitrilelwater 7:3+1% acetic acid): 439 [M+H]⁺.

### Example 73

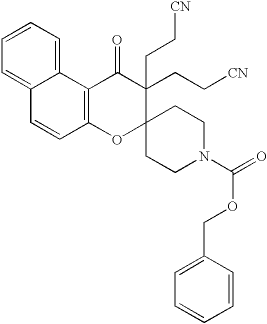

Yield: 20%.

¹H-NMR (400 MHz, 300 K, DMSO-D6, 6/ppm): 1.50-1.80 (m, 2H), 1.80-1.90 (m, 2H), 1.90-2.13 (m, 4H), 2.15-2.30 (m, 2H), 2.30-2.70 (m, 2H), 3.00-3.30 (m, 2H), 3.70-4.00 (m, 2H), 5.05-5.18 (m, 2H), 7.20-7.42 (m, 6H), 7.50 (t, 1H), 7.60-7.70 (m, 1H), 7.90-7.99 (m, 1H), 8.10-8.20 (m, 1H).

MS (DCI/NH₃): 525 [M+NH₄]⁺.

### Example 74

### 3-(2-Cyanoethyl)-7-(3-cyanophenyl)spiro[2H-1-benzopyran-2,1′-cyclobutan]-4(3H)-one

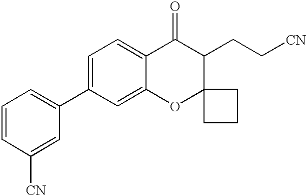

2 ml of 2 N aqueous sodium carbonate solution and 0.01 g (0.01 mmol) of tetrakis(triphenylphosphine)palladium(0) are added to a solution of 0.05 g (0.16 mmol) of the compound from Example 44 and 0.03 g (0.23 mmol) of 3-cyanophenylboronic acid in 10 ml of dioxane under argon, and the mixture is stirred under reflux overnight. After removal of the solvent by distillation, the residue is taken up in dichloromethane/methanol, dried over sodium sulphate and purified by flash chromatography on silica gel (V 4:1). 36.5 mg (62%) of the target compound are obtained.

¹H-NMR (200 MHz, 300 K, DMSO-D₆, δ/ppm):

1.63-2.60 (m, 10H), 2.80 (dd, 1H), 7.42-7.55 (m, 2H), 7.62-7.75 (m, 1H), 7.81 (d, 1H), 7.90 (d, 1H), 8.10 (d, 1H), 8.21-8.30 (m, 1H).

MS (DCI/NH₃): 360 [M+NH₄]⁺.

The following compounds were obtained in analogy to Example 74:

### Example 75

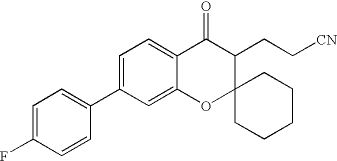

Yield: 38%.

Melting point 61-63° C.; R_{f}: 0.33 (I, 100:1)

MS (DCI): 364 [M+H]⁺.

### Example 76

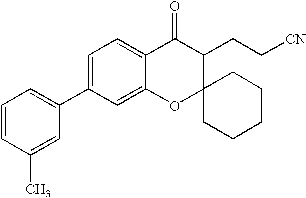

Yield: 42%.

Melting point 86-90° C.; R_{f}: 0.28 (II)

MS (DCI): 360 [M+H]⁺.

### Example 77

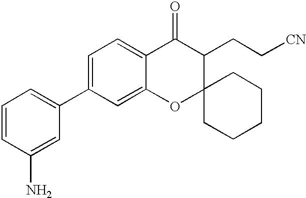

Yield: 34%.

R_{f}: 0.52 (I, 50:1)

¹H-NMR (400 MHz, CDCl₃, δ/ppm): 1.30-1.45 (m, 4H), 1.60 (m, 2H), 1.80 (m, 2H), 2.05 (m, 4H), 2.45-2.55 (m, 3H), 3.75 (br. s, 2), 6.80 (d, 1H), 7.00 (s, 1H), 7.10 (d, 1H), 7.20-7.45 (m, 3H), 7.85 (d, 1H).

MS (DCI): 361 [M+H]⁺.

### Example 78

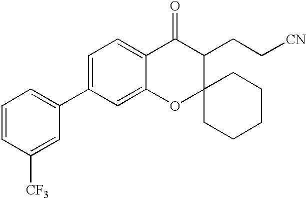

Yield: 58%.

R_{f}: 0.3 (II)

¹H-NMR (400 MHz, CDCl₃, δ/ppm): 1.25-2.10 (m, 12H), 2.45-2.75 (m, 3H), 7.45 (d, 1H), 7.75 (dd, 1H), 7.80 (m, 2H), 8.05 (m, 2H).

MS (DCI): 414 [M+H]⁺.

### Example 79

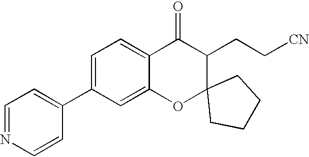

4.50 ml of aqueous 2 M sodium carbonate solution and 0.21 g (0.30 mmol) of bis(triphenylphosphine)palladium(II) chloride are added to a solution of 2.01 g (6.00 mmol) of the compound from Example 52 and 1.11 g (9.00 mmol) of 4-pyridylboronic acid in 30 ml of dimethoxyethane. After 2 h under reflux, a further 1.11 g (9.00 mmol) of 4-pyridylboronic acid and 0.21 g (0.30 mmol) of bis(triphenylphosphine)palladium(II) chloride are added and the mixture is again heated under reflux for 2 h. 100 ml of dimethylformamide, 300 mg of [1,1′-bis(diphenylphosphino)ferrocene]palladium(II) chloride and 300 mg of triphenylphosphine are added to the reaction mixture. It is stirred at 90° C. for 20 h. After cooling, ethyl acetate and 5% strength aqueous NaH₂PO₄ solution are added and, after filtration through Celite, the organic phase is separated off. After the aqueous phase has been extracted with ethyl acetate, the organic phases are washed with 5% strength aqueous NaH₂PO₄ solution, water and aqueous sodium chloride solution and dried over magnesium sulphate, the solvent is removed by distillation, and the residue is purified by chromatography under silica gel (V 2:1). Subsequent normal HPLC purification (V 35:65) and recrystallization from diethyl ether afford 0.805 g (40.36%) of the target compound in the form of colourless crystals.

¹H-NMR (200 MHz, DMSO-D₆, δ/ppm): 1.55-2.20 (m, 10H), 2.45-2.55 (m, 2H), 2.60 (dd, 1H), 7.15 (d, 1H), 7.25 (d, 1H), 7.50 (dd, 2H), 7.92 (d, 1H), 8.60-8.80 (m,2H).

MS (ESI acetonitrile/water 7:3+0.1% acetic acid): 333 [M+H]⁺.

### Example 80

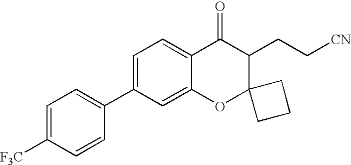

Yield: 75%.

¹H-NMR (200 MHz, 300 K, DMSO-D₆, δ/ppm):

1.62-2.20 (m, 6H), 2.20-2.42 (m, 2H), 2.52-2.60 (m, 2H), 2.80 (dd, 1H), 7.40-7.51 (m, 2H), 7.80-7,91 (m, 3H), 7.91-8.05 (m, 2H).

MS (DCI/NH₃): 403 [M+NH₄]⁺.

### Example 81

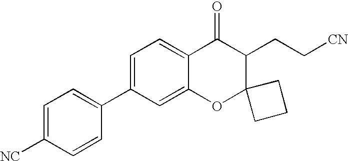

Yield: 60%.

¹H-NMR (200 MHz, 300 K, DMSO-D₆, δ/ppm):

1.64-2.24 (m, 6H), 2.25-2.42 (m, 2H), 2.50-2.60 (m, 2H), 2.80 (dd, 1H), 7.42-7.51 (m, 2H), 7.80-7.88 (m,1H), 7.95 (s, 4H).

MS (DCI/NH₃): 360 [M+NH₄]⁺.

### Example 82

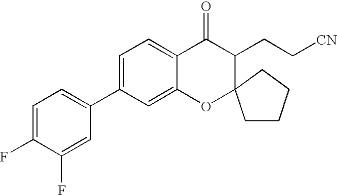

8.44 ml of 2 M aqueous sodium carbonate solution and 0.49 g (0.70 mrnol) of bis(triphenylphosphine)palladium(II) chloride are added to a solution of 4.70 g (14.06 mmol) of the compound from Example 52 and 2.66g (16.88 mmol) of 3,4-difluorophenylboronic acid in 85 ml of dimethoxyethane. Stirring under reflux for 2 hours is followed by addition of ethyl acetate, washing with 5% strength aqueous sodium dihydrogen phosphate solution and drying over magnesium sulphate. The solvent is removed by distillation and the residue is purified by chromatography on silica gel (VI 1:1, V 10:1) and recrystallized from diethyl ether and diethyl ether/n-pentane. The product is obtained in the form of colourless crystals in a yield of 4.52 g (87%).

¹H-NMR (200 MHz, CDCl₃, δ/ppm): 1.55-2.25 (m, 10H), 2.45-2.50 (m, 2H), 2.55 (dd, 1H), 7.05 (d, 1H), 7.10-7.50 (m, 4H), 7.90 (d, 1H).

MS (ESI acetonitrile/water 7:3+0.1% acetic acid): 368 [M+H]⁺.

R_{f}: 0.48 (cyclohexane/ethyl acetate 2:1).

The following compounds were obtained in analogy to Example 82:

### Example 83

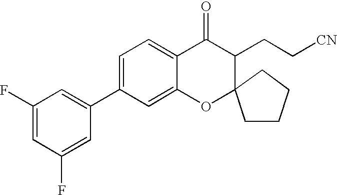

Yield: 88%.

¹H-NMR (400 MHz, 300 K, CDCl₃, δ/ppm): 1.60-2.20 (m,10H), 2.35-2.53 (m, 2H), 2.55 (dd, 1H), 6.80-6.90 (m, 1H), 7.06-7.15 (m, 3H), 7.20 (dd, 1H), 7.90 (d, 1H).

MS (ESI acetonitrile/water 7:3+0.1% acetic acid): 368 [M+H]⁺, 390 [M+Na]⁺.

### Example 84

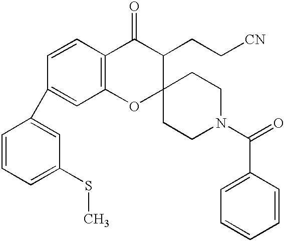

Yield: 33%.

¹H-NMR (400 MHz, 300 K, CD₂Cl₂, δ/ppm): 1.50-2.20 (m, 6H), 2.40-2.70 (m, 6H), 3.00-3.60 (m, 3H), 4.40-4.70 (m, 1H), 7.11-7.19 (m, 3H), 7.22-7.31 (m, 7H), 7.33-7.37 (m, 1H), 7.74 (d, 1H).

MS (ESI, acetonitrile/water 7:3+1% acetic acid): 497 [M+H]⁺.

### Example 85

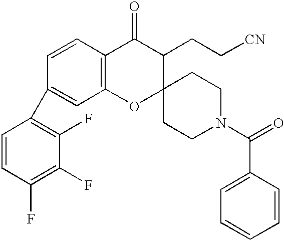

Yield: 72%.

¹H-NMR (400 MHz, 300 K, CD₂Cl₂, δ/ppm):

1.50-2.30 (m, 6H), 2.40-2.70 (m, 3H), 3.10-3.70 (m, 3H), 4.40-4.70 (m, 1H), 7.00-7.30 (m, 4H), 7.38-7.48 (m, 5H), 7.91 (d, 1H).

MS (ESI, acetonitrile/water 7:3+1% acetic acid): 505 [M+H]⁺.

### Example 86

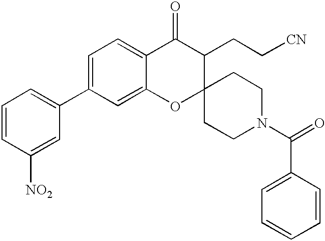

Melting point 89-93° C.; yield: 67%.

### Example 87

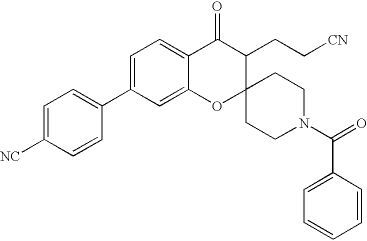

Melting point 120-128° C.; yield: 57%.

### Example 88

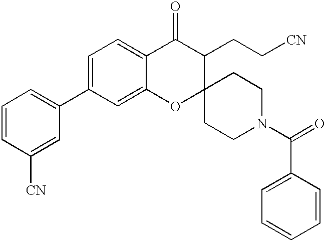

Melting point 91-99° C.; yield: 58%.

### Example 89

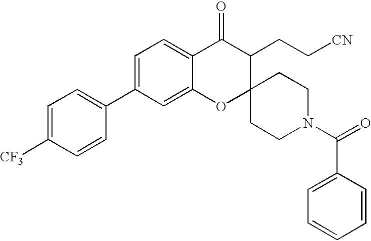

Yield: 68%.

¹H-NMR (400 MHz, 300 K, CD₂Cl₂, δ/ppm):

1.40-2.10 (m, 7H), 2.20-2.50 (m, 3H), 2.90-3.60 (m, 3H), 7.10-7.15 (m, 2H), 7.18-7.26 (m, 5H), 7.57 (s, 4H), 7.74 (d, 1H).

MS (ESI, acetonitrile/water 7:3+1% acetic acid): 519 [M+H]⁺

### Example 90

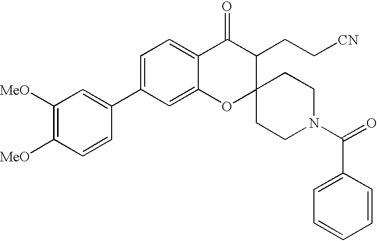

Melting point 88-92° C.; yield: 74%.

### Example 91

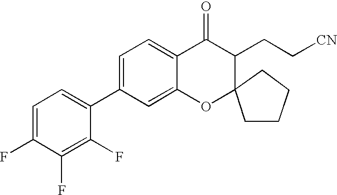

Yield: 68%.

¹H-NMR (400 MHz, 300 K, CD₂Cl₂, δ/ppm): 1.60-2.20 (m, 10H), 2.34-2.53 (m, 2H), 2.55 (dd, 1H), 7.05-7.25 (m, 4H), 7.90 (d, 1H).

MS (ESI acetonitrile/water 7:3+0.1% acetic acid): 386[M+H]⁺, 408 [M+Na]⁺.

### Example 92

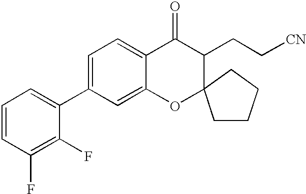

Yield: 83%.

¹H-NMR (400 MHz, 300 K, CD₂Cl₂, δ/ppm): 1.60-1.95 (m, 6H), 1.95-2.20 (m, 4H), 2.35-2.55 (m, 2H), 2.60 (dd, 1H), 7.10 (s, 1H), 7.15-7.30 (m, 5H), 7.90 (d,1H).

MS (ESI acetonitrile/water 7:3+0.1% acetic acid): 368 [M+H]⁺, 390 [M+Na]⁺.

### Example 93

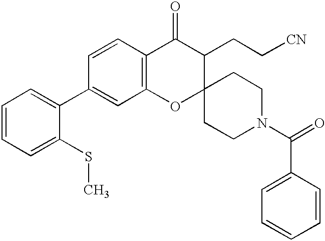

Yield: 17%.

¹H-NMR (400 MHz, 300 K, CD₂Cl₂, δ/ppm): 4.20-4.70 (m, 6H), 4.90 (s, 3H), 5.00-5.10 (m, 1H), 5.10-5.22 (m, 2H), 5.30-5.38 (m, 2H), 5.50-5.80 (m, 1H), 6.00-6.20 (m, 1H), 9.55-9.65 (m, 2H), 9.67-9.75 (m, 2H), 9.83-9.97 (m, 6H), 10.28 (d, 1H), 10.45 (s, 1H).

MS (ESI, acetonitrile/water 7:3+1% acetic acid): 497 [M+H]⁺.

### Example 94

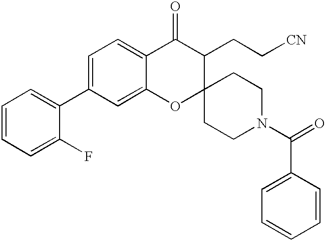

Yield: 29%.

¹H-NMR (400 MHz, 300 K, CD₂Cl₂, δ/ppm): 1.70-1.90 (m, 2H), 1.90-2.30 (m, 4H), 2.40-2.61 (m, 2H), 2.61-2.70 (m, 1H), 3.10-3.30 (m, 3H), 7.15-7.30 (m, 4H), 7.30-7.52 (m, 7H), 7.90 (d, 1H).

MS (ESI acetonitrile/water 7:3+0.1% acetic acid): 469 [M+H]⁺, 491 [M+Na]⁺.

### Example 95

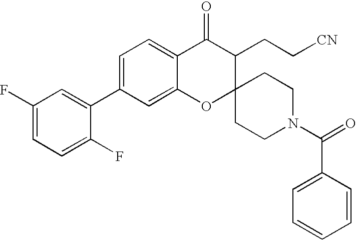

Yield: 45%.

¹H-NMR (400 MHz, 300 K, CD₂Cl₂, δ/ppm):

1.40-2.20 (m, 7H), 2.30-2.62 (m, 3H), 3.00-3.20 (m, 1H), 3.30-3.70 (m, 2H), 6.98-7.20 (m, 5H), 7.28-7.39 (m, 5H), 7.82 (d, 1H).

MS (ESI, acetonitrile/water 7:3+1% acetic acid): 487 [M+H]⁺.

### Example 96

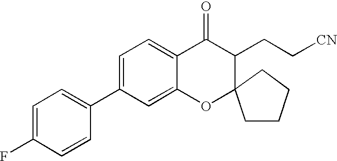

Yield: 71%.

¹H-NMR (300 MHz, 300 K, CDCl₃, δ/ppm): 1.55-2.20 (m, 10H), 2.35-2.50 (m, 2H), 2.55 (dd, 1H), 7.10-7.20 (m, 4H), 7.53-7.62 (m, 2H), 7.90 (d, 1H).

MS (ESI acetonitrile/water 7:3+0.1% acetic acid): 350 [M+H]⁺, 372 [M+Na]⁺.

### Example 97

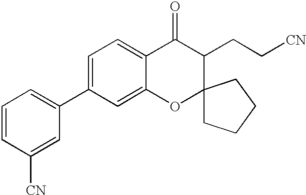

Yield: 70%.

¹H-NMR (300 MHz, 300 K, CDCl₃, δ/ppm):

1.60-2.20 (m, 10H), 2.36-2.55 (m, 2H), 2.60 (dd, 1H), 7.11 (d, 1H), 7.20 (d, 1H), 7.53-7.62 (m, 1H), 7.68 (dd, 1H), 7.81 (dd,l H), 7.88 (m, 1H), 7.95 (d, 1H).

MS (ESI acetonitrile/water 7:3+0.1% acetic acid): 357 [M+H]⁺, 379 [M+Na]⁺.

### Example 98

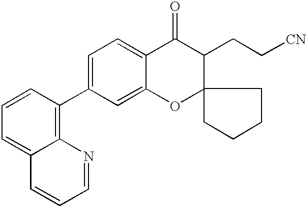

Yield: 69%.

¹H-NMR (400 MHz, 300 K, CD₂Cl₂, δ/ppm): 1.61-1.99 (m, 6H), 2.01-2.20 (m, 4H), 2.38-2.61 (m, 3H), 7.25 (d, 1H), 7.35 (dd, 1H), 7.42-7.50 (m, 1H), 7.60-7.65 (m, 1H), 7.75 (dd, 1H), 7.85-7.95 (m, 2H), 8.25 (dd, 1H), 8.90 (dd, 1H).

MS (ESI acetonitrile/water 7:3+0.1% acetic acid): 383 [M+H]⁺.

### Example 99

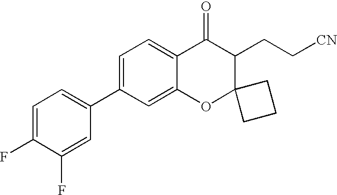

Yield: 81%.

¹H-NMR (300 MHz, 300 K, CDCl₃, δ/ppm): 1.70-2.58 (m, 10H), 2.71 (dd, 1H), 7.12 (d, 1H), 7.18 (dd, 1H), 7.20-7.29 (m, 1H), 7.29-7.35 (m, 1H), 7.35-7.45 (m, 1H), 7.90 (d, 1H)

MS (ESI acetonitrile/water 7:3+0.1% acetic acid): 354 [M+H]⁺, 376 [M+Na]⁺.

### Example 100

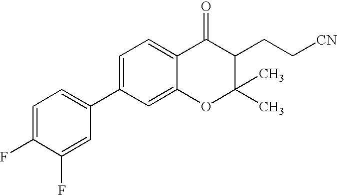

Yield: 65%.

¹H-NMR (300 MHz, 300 K, CDCl₃, δ/ppm): 1.35 (s, 3H), 1.55 (s, 3H), 1.82-1.95 (m, 1H), 1.98-2.12 (m, 1H), 2.50-2.70 (m, 2H), 2.71 (dd, 1H), 7.05 (d, 1H), 7.15 (dd, 1H), 7.20-7.45 (m, 3H), 7.88 (d, 1H).

MS (ESI acetonitrile/water 7:3+0.1% acetic acid): 342 [M+H]⁺, 683 [2M+H]⁺.

### Example 101

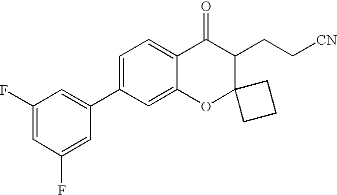

Yield: 78%.

¹H-NMR (300 MHz, 300 K, CDCl₃, δ/ppm): 1.72-2.57 (m, 10H), 2.72 (dd, 1H), 6.82-6.90 (m, 1H), 7.05-7.13 (m, 2H), 7.15 (d, 1H), 7.20 (dd, 1H), 7.90 (d, 1H).

MS (ESI acetonitrile/water 7:3+0.1% acetic acid): 354[M+H]⁺, 376 [M+Na]⁺.

### Example 102

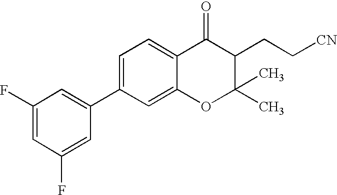

Yield: 70%.

¹H-NMR (200 MHz, 300 K, CDCl₃, δ/ppm): 1.38 (s, 3H), 1.60 (s, 3H), 1.80-2.20 (m, 2H), 1.95-2.70 (m, 2H), 2.75 (dd, 1H), 6.78-6.95 (m,1H), 7.02-7.20 (m, 4H), 7.90 (d, 1H).

MS (DCI/NH₃): 341 [M]+, 359 [M+NH₄]⁺.

### Example 103

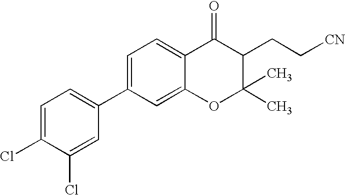

Yield: 83%.

¹H-NMR (200 MHz, 300 K, CDCl₃, δ/ppm): 1.35 (s, 3H), 1.58 (s, 3H), 1.80-2.18 (m, 2H), 2.54-2.69 (m, 2H), 2.72 (dd, 1H), 7.10 (d, 1H), 7.18 (dd, 1H), 7.38-7.46 (m, 1H), 7.70 (d, 1H), 7.89 (d, 1H).

MS (DCI/NH₃): 391 [M+NH₄]⁺.

### Example 104

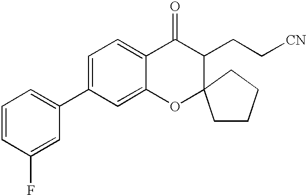

Yield: 79%.

¹H-NMR (400 MHz, 300 K, CD₂Cl₂, δ/ppm): 1.60-2.20 (m, 10H), 2.35-2.55 (m, 2H), 2.55 (dd, 1H), 7.05-7.15 (m, 2H), 7.22 (dd, 1H), 7.31 (dd, 1H), 7.40-7.50 (m, 2H), 7.85-7.95 (m, 1H).

MS (ESI acetonitrile/water 7:3+0.1% acetic acid): 350[M+H]⁺, 372 [M+Na]⁺.

### Example 105

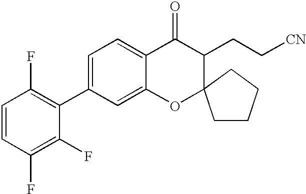

Yield: 43%.

¹H-NMR (400 MHz, 300 K, CD₂Cl₂, δ/ppm): 1.60-1.95 (m, 6H), 1.95-2.20 (m, 4H), 2.35-2.55 (m, 2H), 2.60 (dd, 1H), 6.92-7.05 (m, 1H), 7.05 (s, 1H), 7.10 (dd, 1H), 7.15-7.30 (m, 1H), 7.90 (d, 1H).

MS (ESI acetonitrile/water 7:3+0.1% acetic acid): 386 [M+H]⁺, 408 [M+Na]⁺.

### Example 106

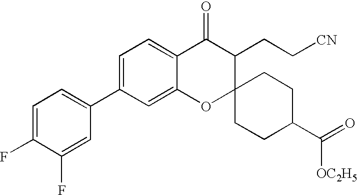

Yield: 56%.

¹H-NMR (400 MHz, 300 K, CD₂Cl₂, δ/ppm):

1.15 (t, 3H), 1.60-1.70 (m, 1H), 1.75-1.95 (m, 9H), 2.25-2.45 (m, 2H), 2.50-2.58 (m, 2H), 4.04 (q, 2H), 7.07 (d, 1H),7.11 (dd, 1H), 7.16-7.24 (m, 1H), 7.26-7.32 (m, 1H), 7.33-7.41 (m, 1H), 7.78 (d, 1H).

MS (ESI, acetonitrile/water 7:3+1% acetic acid): 454 [M+H]⁺.

### Example 107

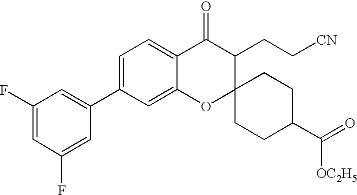

Yield: 76%.

¹H-NMR (400 MHz, 300 K, CD₂Cl₂, δ/ppm):

1.50 (t, 3H), 1.94-2.05 (m, 1H), 2.08-2.30 (m, 9H), 2.60-2.80 (m, 2H), 2.85-2.92 (m, 2H), 4.38 (q, 2H), 7.09-7.17 (m, 1H), 7.38-7.49 (m, 4H), 8.13 (d, 1H).

MS (ESI, acetonitrile/water 7:3+1% acetic acid): 454 [M+H]⁺

### Example 108

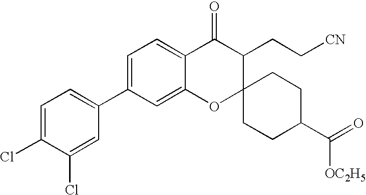

Yield: 31%.

¹H-NMR (400 MHz, 300 K, CD₂Cl₂, δ/ppm):

1.25 (t, 3H), 1.70-1.80 (m, 1H), 1.80-2.05 (m, 9H), 2.38-2.58 (m, 2H), 2.60-2.68 (m, 2H), 4.15 (q, 2H), 7.20 (d, 1H), 7.22 (dd, 1H), 7.49 (dd, 1H), 7.57 (d, 1H), 7.75 (d, 1H), 7.88 (d, 1H).

MS (ESI, acetonitrile/water 7:3+1% acetic acid): 486 [M+H]⁺.

### Example 109

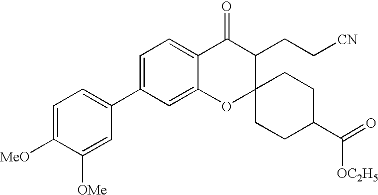

Melting point 71-73° C.; yield: 14%.

### Example 110

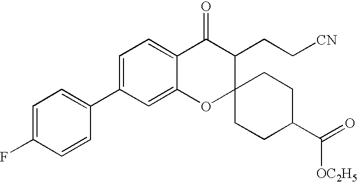

Yield: 11%.

¹H-NMR (400 MHz, 300 K, CD₂Cl₂, δ/ppm):

1.18 (t, 3H), 1.62-1.70 (m, 1H), 1.75-1.98 (m, 9H), 2.28-2.45 (m, 2H), 2.53-2.59 (m, 2H), 4.05 (q, 2H), 7.05-7.17 (m, 4H), 7.51-7.57 (m, 2H), 7.77 (d, 1H).

MS (DCI/NH₃): 453 [M+NH₄]⁺.

### Example 111

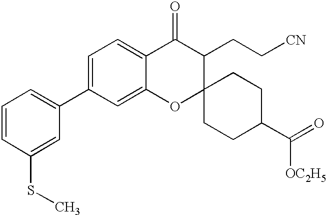

Yield: 33%.

¹H-NMR (400 MHz, 300 K, CD₂Cl₂, δ/ppm):

1.27 (t, 3H), 1.70-1.80 (m,l H), 1.83-2.05 (m, 9H), 2.38-2.52 (m, 2H), 2.62-2.68 (m, 2H), 4.15 (q, 2H), 7.21 (d, 1H), 7.26 (dd, 1H), 7.28-7.34 (m, 1H), 7.37-7.42 (m, 2H), 7.49-7.52 (m, 1H), 7.87 (d, 1H).

MS (DCI/NH₃): 481 [M+NH₄]⁺.

### Example 112

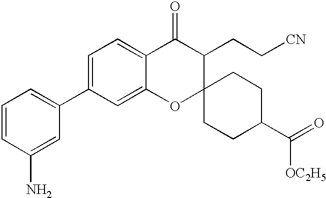

Yield: 24%.

¹H-NMR (400 MHz, 300 K, CD₂Cl₂, δ/ppm):

1.35 (t, 3H), 1.80-1.88 (m, 1H), 1.90-2.12 (m, 9H), 2.43-2.63 (m, 2H), 2.63-2.76 (m, 2H), 4.22 (q, 2H), 6.80-6.85 (m, 1H), 7.00-7.05 (m, 1H), 7.05-7.11 (m, 2H), 7.26 (s, 1H), 7.31 (d, 2H), 7.91 (d, 1H).

MS (DCI/NH₃): 450 [M+NH₄]⁺.

### Example 113

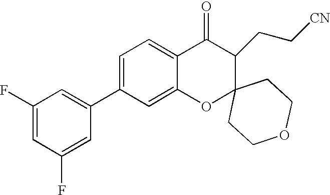

Yield: 47%.

¹H-NMR (400 MHz, 300 K, CDCl₃, δ/ppm): 1.65-1.80 (m, 1H), 1.84-2.12 (m, 5H), 2.43-2.59 (m, 2H), 2.60-2.67 (m, 1H), 3.75-3.90 (m, 4H), 6.82-6.92 (m, 1H), 7.10-7.16 (m, 2H), 7.21-7.25 (m, 2H), 7.92 (d, 1H).

MS (ESI acetonitrile/water 7:3+0.1% acetic acid): 384 [M+H]⁺.

### Example 114

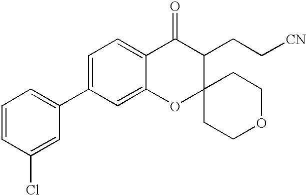

Yield: 52%.

¹H-NMR (300 MHz, 300 K, DMSO-D₆, δ/ppm): 1.65-1.95 (m, 4H), 2.00-2.13 (m, 1H), 2.35-2.65 (m, 3H), 2.70 (dd, 1H), 3.60-3.80 (m, 4H), 7.40-7.56 (m, 4H), 7.70-7.75 (m, 1H), 7.76-7.88 (m, 2H).

MS (EI POS): 381 [M]⁺.

### Example 115

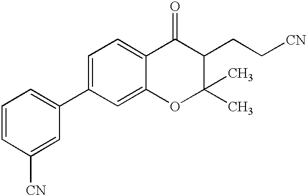

Yield: 60%.

¹H-NMR (400 MHz, 300 K, CD₂Cl₂, δ/ppm): 1.38 (s, 3H), 1.58 (s, 3H), 1.85-1.95 (m, 1H), 1.96-2.10 (m, 1H), 2.50-2.60 (m, 1H), 2.61-2.70 (m, 1H), 2.72 (dd, 1H), 7.07 (d, 1H), 7.14 (dd, 1H), 7.51 (t, 1H), 7.62 (td, 1H), 7.75-7.84 (m, 3H).

MS (DCO₃):348 [M+NH₄]⁺.

### Example 116

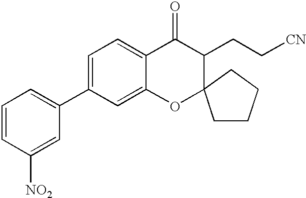

Yield: 83%.

¹H-NMR (400 MHz, 300 K, CD₂Cl₂, δ/ppm):

1.53-2.11 (m, 10H), 2.30-2.47 (m, 2H), 2.52 (dd, 1H), 7.16 (s, 1H), 7.22 (d, 1H), 7.59 (t, 1H), 7.83-7.91 (m, 2H), 8.17 (d, 1H), 8.36-8.42 (m, 1H).

MS (ESI, acetonitrile/water 7:3+1% acetic acid): 377 [M+H]⁺.

### Example 117

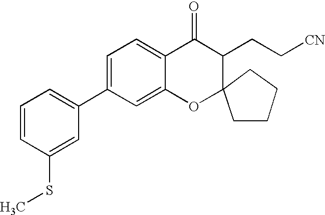

Yield: 51%.

¹H-NMR (400 MHz, 300 K, CD₂Cl₂, δ/ppm):

1.52-2.08 (m, 11H), 2.26-2.50 (m, 5H), 7.07 (d, 1H), 7.16 (dd, 1H), 7.19-7.24 (m, 1H), 7.27-7.32 (m, 2H), 7.39-7.42 (m, 1H).

MS (ESI, acetonitrile/water 7:3+1% acetic acid): 378 [M+H]⁺.

### Example 118

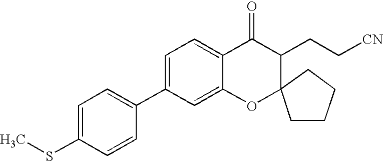

Melting point 104.8° C.; yield: 56%.

### Example 119

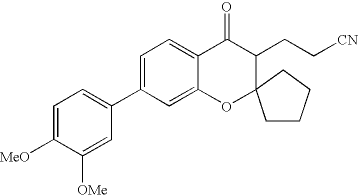

Yield: 59%. H-NMR (400 MHz, 300 K, CD₂Cl₂, δ/ppm):

1.60-2.19 (m, 10H), 2.35-2.58 (m, 3H), 3.89 (s, 3H), 3.91 (s, 3H), 6.97 (d, 1H), 7.15 (t, 2H), 7.20-7.27 (m, 2H), 7.86 (d, 1H).

MS (ESI, acetonitrile/water 7:3+1% acetic acid): 392 [M+H]⁺

### Example 120

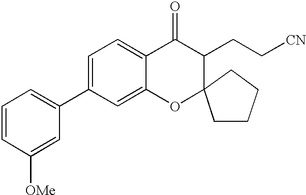

Yield: 61%.

¹H-NMR (400 MHz, 300 K, CD₂Cl₂, δ/ppm):

1.52-2.08 (m, 10H), 2.27-2.50 (m, 3H), 3.78 (s, 3H), 6.87 (ddd, 1H), 7.06 (t, 1H), 7.08 (d, 1H), 7.10-7.15 (m, 1H), 7.17 (dd, 1H), 7.30 (t, 1H), 7.79 (d, 1H).

MS (ESI, acetonitrile/water 7:3+1% acetic acid): 362 [M+H]⁺.

### Example 121

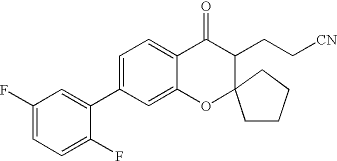

Melting point 72° C.; yield: 23%.

### Example 122

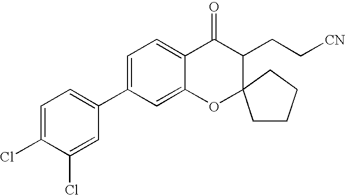

Melting point 97.7° C.; yield: 71%.

### Example 123

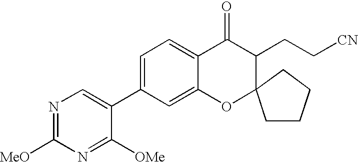

Melting point 112° C.; yield: 28%.

### Example 124

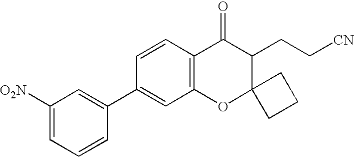

Melting point 138.8° C.; yield: 30%.

### Example 125

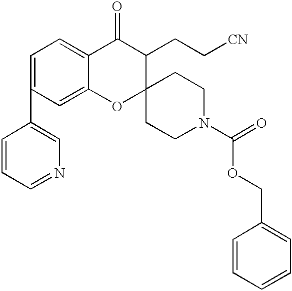

Yield: 12.63%.

¹H-NMR (400 MHz, 300 K, CD₂Cl₂, δ/ppm): 1.40-1.62 (m, 2H), 2.00-2.12 (m, 2H), 2.12-2.30 (m, 2H), 2.35-2.45 (m, 2H), 3.40-3.62 (m, 2H), 3.95-4.00 (m, 2H), 4.22-4.30 (m, 1H), 5.08 (s, 2H), 7.12-7.45 (m, 8H), 7.95 (d, 2H), 8.60-8.65 (m, 1H), 8.82-8.90 (m, 1H).

MS (DCI/NH₃): 482 [M+H]⁺.

### Example 126

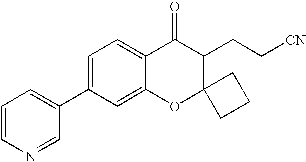

Yield: 28.41%.

¹H-NMR (400 MHz, 300 K, CD₂Cl₂, δ/ppm): 1.70-2.52 (m, 12H), 2.75 (dd, 1H), 7.25-7.30 (m, 2H), 7.58-7.70 (m, 1H), 7.95 (d, 1H), 8.10-8.21 (m, 1H), 8.60-8.72 (m, 1H), 8.85-8.95 (m, 1H).

MS (ESI acetonitrile/water 7:3+0.1% acetic acid): 319 [M+H]⁺, 341 [M+Na]⁺.

### Example 127

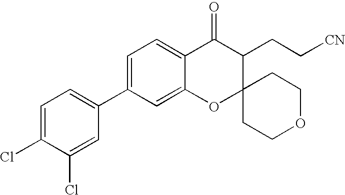

Melting point 145-149° C.; yield: 32%.

### Example 128

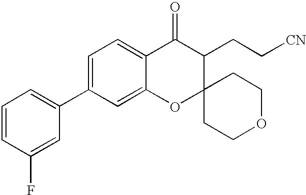

Melting point 103-105° C.; yield: 45%.

### Example 129

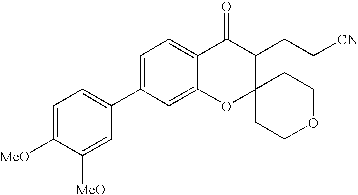

Yield: 57%.

¹H-NMR (200 MHz, 300 K, CDCl₃, δ/ppm):

1.60-2.10 (m, 6H), 2.45-2.65 (m, 3H), 3.76-3.92 (m, 4H), 3.94 (s, 3H), 3.97 (s, 3H), 6.91-7.00 (d, 1H), 7.12 (d, 1H), 7.16-7.29 (m, 3H), 7.88 (d, 1H).

MS (ESI, acetonitrile/water 7:3+1% acetic acid): 408 [M+H]⁺.

### Example 129A

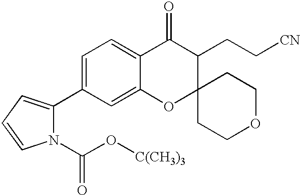

Yield: 26%.

¹H-NMR (200 MHz, 300 K, CDCl₃, δ/ppm): 1.40 (s, 9H), 1.60-2.10 (m, 6H), 2.45-2.65 (m, 3H), 3.70-3.90 (m, 4H), 6.20-6.35 (m, 2H), 6.95-7.05 (m, 2H), 7.35-7.40 (m, 1H), 7.81 (d, 1H).

MS (ESI, acetonitrile/water 7:3+0.1% acetic acid): 437 [M+H]⁺, 459 [M+Na]⁺.

### Example 130

### 3-(2-Carboxy)-7-phenylspiro[2H-1-benzopyran-2,1′-cyclohexan]-4(3H)-one

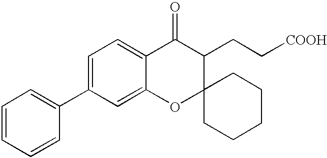

A mixture of 100 mg (0.25 mmol) of the compound from Example 47, 0.35 ml of 1 N sodium hydroxide solution, 2 ml of ethanol and 2 ml of water is stirred at 50° C. for two hours. The mixture is adjusted to pH 7 with dilute hydrochloric acid and evaporated to dryness. The residue is stirred with isopropanol, filtered off with suction and dried.

Yield: 58 mg (63%)

Melting point: 196° C.

MS (DCI): 365 [M+H]⁺

R_{f}: 0.32 (I, 20:1).

### Example 131

### 3-(2-Cyanoethyl)-7-(3-pyridyl)spiro[2H-1-benzopyran-2,1′-cyclohexan]-4(3H)-one Hydrochloride

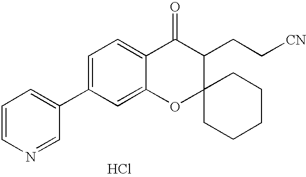

20 mg of the compound from Example 49 are stirred in 5 ml of 4 M hydrochloric acid solution in dioxane at room temperature overnight. All the volatile components are removed under reduced pressure, and the residue is stirred with isopropanol, filtered off with suction and dried.

Yield: 14 mg (64%)

### Example 132

### 3-(2-Cyanoethyl)-7-(3-aminophenyl)spiro[2H-1-benzopyran-2,1′-cyclohexan]-4(3H)-one Hydrochloride

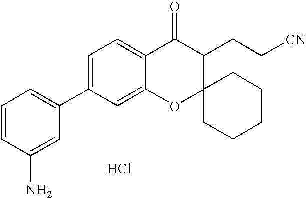

The title compound is obtained on reaction of the compound from Example 77 with dioxane/hydrochloric acid in analogy to Example 131.

Yield: 60%.

### Example 133

### 3-(2-Cyanoethyl)-6-fluoro-7-(4-phenylpiperazin-1-yl)spiro[2H-1-benzopyran-2,1 ′-cyclohexan]-4(3H)-one

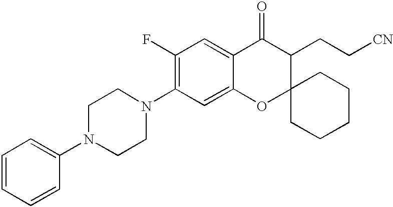

A solution of 100 mg (0.33 mmol) of the compound from Example 51 and 160 mg (0.99 mmol) of phenylpiperazine in 3 ml of DMSO is kept at 1 00° C. for 3 hours. The solvent is removed by distillation, and the residue is mixed with water and extracted with dichloromethane. The organic phase is washed with water and dried over sodium sulphate, and the solvent is removed. The crude product is stirred with petroleum ether and isolated.

| | |
|---|---|
| Yield: | 110 mg (75%) |
| Melting point: | 163-168° C. |
| MS (DCI): 448 [M + H]⁺ | |
| R_{f}: | 0.5 (I, 100:1). |

### Example 134

### 3-[7-(4-Aminocarbonylphenyl)spiro[2H-1-benzopyran-2,1′-cyclobutan]-4(3H)-on-3-yl]-propionamide

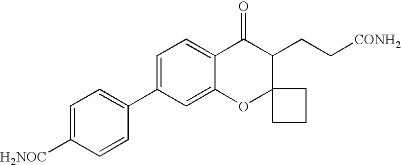

While cooling in ice, 14.69 mg (0.11 mmol) of potassium carbonate and 24.11 mg (0.02 ml, 0.21 mmol) of hydrogen peroxide in 2.00 ml of DMSO are added to 18.20 mg (0.05 mmol) of the compound from Example 81, and the mixture is stirred at room temperature for 3 hours. Then, once again, the same amounts of hydrogen peroxide and potassium carbonate are added while cooling in ice. After stirring at room temperature for 2 hours, water and ethyl acetate are added, and the organic phase is separated off, washed with water and saturated aqueous sodium chloride solution and dried over sodium sulphate. Chromatographic purification on silica gel (I 10:1) affords 15.1 mg (75%) of colourless crystals.

¹H-NMR (400 MHz, DMSO-D₆, δ/ppm): 1.50-1.54 (m, 1H), 1.70-1.82 (m, 1H), 1.85-2.25 (m, 6H), 2.30-2.45 (m, 2H), 2.65 (dd, 1H), 6.72 (br.s, 1H), 7.25 (br.s, 1H), 7.40-7.50 (m, 2H), 7.75-7.90 (m, 2H), 7.95 (d, 2H), 8.05 (s, 1H).

MS (ESI acetonitrile/water 7:3+0.1% acetic acid): 379 [M+H]⁺, 401 [M+Na]+, 779 [2M+Na]⁺.

The following compounds were obtained in analogy to Example 134:

### Example 135

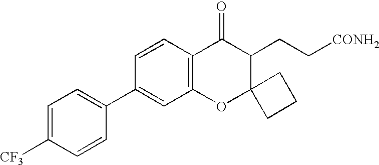

Yield: 31%

MS (DCI/NH₃): 404 [M+H]⁺.

HPLC (XII): RT=4.98.

### Example 136

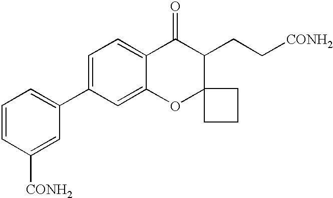

Yield: 50%.

¹H-NMR (200 MHz, 300 K, DMSO-D₆, δ/ppm): 1.20-2.20 (m, 8H), 2.30-2.45 (m, 2H), 2.65 (dd, 1H), 6.75 (br. s, 2H), 7.30 (br.s, 2H), 7.40-7.50 (m, 2H), 7.51-7.62 (m, 1H), 7.75-7.85 (m, 1H), 7.85-7.95 (m, 1H), 8.10-8.20 (m, 1H), 8.20-8.30 (m, 1H).

MS (DCI/NH₃): 379 [M+H]⁺.

### Example 137

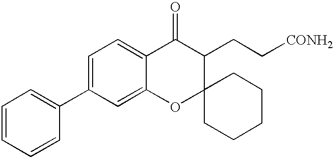

¹H-NMR (200 MHz, 300 K, DMSO-D₆, δ/ppm)=1.35-2.15 (m, 15H), 6.78 (br. s 1H), 7.27 (br. s, 1H), 7.31 (s, 1H), 7.36 (d, 1H), 7.48 (m, 3H), 7.77 (m, 3H).

MS (DCI/NH₃): 364 [M+H]⁺

### Example 138

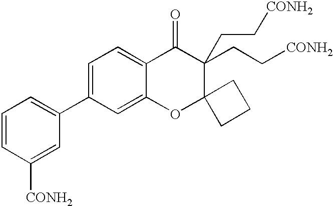

Yield: 36%.

¹H-NMR (400 MHz, 300 K, DMSO-D₆, δ/ppm):

1.78-1.90 (m, 3H), 1.95-2.27 (m, 9H), 2.39-2.48 (m, 2H), 6.77 (br. s, 2H), 7.38 (br. s, 2H), 7.43-7.50 (m, 3H), 7.58 (t, 1H), 7.79 (d, 1H), 7.90-7.95 (m, 2H), 8.18 (br. s, 1H),8.24(br.S, 1H).

MS (ESI, acetonitrile/water 7:3+1% acetic acid): 450 [M+H]⁺, 472 [M+Na]⁺.

### Example 139

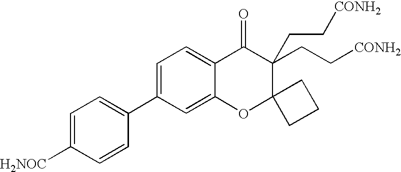

Yield: 84%.

¹H-NMR (200 MHz, 300 K, DMSO-D₆, δ/ppm):

1.70-2.30 (m, 11H), 2.34-2.50 (m, 3H), 6.80 (br. s, 2H), 7.34 (br. s, 2H), 7.40-7.50 (m, 2H), 7.75-7.90 (m, 3H), 7.98 (d, 2H), 8.07 (br. s, 2H).

MS (ESI, acetonitrile/water 7:3+1% acetic acid): 450 [M+H]⁺.

### Example 140

### 3-(2-Cyanoethyl)-7-(4-aminophenyl)spiro[2H-1-benzopyran-2,1′-4-azacyclohexan]-4(3H)-one

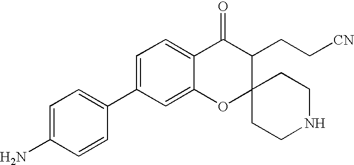

0.03 g (0.05 mmol) of the compound from Example 57 is dissolved in 2 ml of an ethyl acetate/methanol (1:1) mixture, and 0.12 ml of water and 0.02 ml of concentrated hydrochloric acid are added. Hydrogenation is then carried out under atmospheric pressure in the presence of 0.061 g of palladium on activated carbon (Pd/C 10%). After removal of the catalyst and removal of the solvent by distillation, the residue is purified by HPLC (XIV). The target compound is obtained in a yield of 3.8 mg (21.34%).

¹H-NMR (400 MHz, DMSO-D₆, δ/ppm): 1.65-2.10 (m, 6H), 2.30-2.45 (m, 1H), 2.50-2.60 (m, 1H), 2.65 (dd, 1H), 3.00-3.22 (m, 4H), 6.60 (d, 2H), 7.20-7.30 (m, 2H), 7.40 (d, 2H), 7.65 (d, 1H), 8.10-8.30 (m, 1H), 8.50-8.65 (m, 1H).

MS (ESI acetonitrile/water 7:3+0.1% acetic acid): 362 [M+H]⁺, 384 [M+Na]⁺.

### Example 141

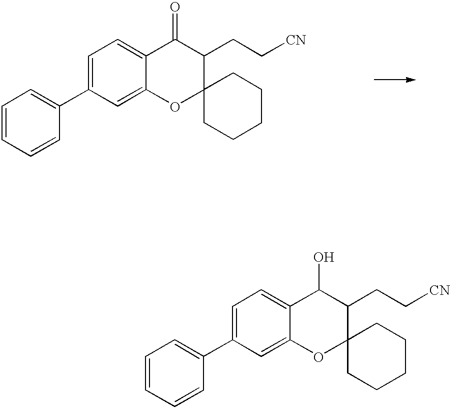

At −78° C., 5.5 mg (0.14 mmol) of sodium borohydride are added to a solution of 50 mg (0.14 mmol) of the compound from Example 40 of EP-A 4 624 in 2 ml of methanol, and the reaction mixture is stirred at this temperature for 2 hours. After addition of a further 0.28 nunol of sodium borohydride, the mixture is stirred at room temperature until the precursor has reacted completely. Water is then added, and the crystals are filtered off with suction and dried. The target compound is obtained in a yield of 33.8 mg (67.21%).

¹H-NMR (200 MHz, DMSO-D₆, δ/ppm):

1.3-1.85 (m, 12H), 2.09 (m, 1H), 2.6-2.78 (m, 2H), 4.75 (dd, 1H), 5.60 (d, 1H, OH), 7.04 (s, 1H), 7.21 (d, 1H), 7.37 (dd, 1H), 7.43 (m, 3H), 7.63 (d, 2H).

MS (DCI, NH₃): 365 [M+NH₄]⁺.

Description of Tests

The antiviral action of the compounds according to the invention on hepatitis B virus was investigated by methods based on those described by M. A. Sells et al., Proc. Natl. Acad. Sci. 84, 1005-1009 (1987) and B. E. Korba et al., Antiviral Research 19, 55-70 (1992).

The antiviral tests were carried out in 96-well microtitre plates. The first vertical row of the plate received only growth medium and HepG2.2.15 cells. It served as virus control.

Stock solutions of the test compounds (50 mM) were initially dissolved in DMSO, and further dilutions were prepared in the HepG2.2.15 growth medium. The compounds according to the invention were usually pipetted in a test concentration of 100 μM (lst test concentration) in each case into the second vertical test row of the microtitre plate and subsequently diluted in twofold steps 2¹⁰ times in growth medium +2% by weight fetal calf serum (volume 25 μl).

Each well of the microtitre plate then contained 225 μl of HepG2.2.15 cell suspension (5×10⁴ cells/ml) in growth medium +2% by weight of fetal calf serum. The test mixture was incubated at 37° C. and 5% CO₂ (v/v) for 4 days.

The supernatant was then aspirated off and discarded, and the wells received 225 μl of freshly prepared growth medium. The compounds according to the invention were each added anew as 10-fold concentrated solution in a volume of 25 μl. The mixtures were incubated for a further 4 days.

Before harvesting the supernatants and/or cells to determine the antiviral effect, the HepG2.2.15 cells were examined under the light microscope or by means of biochemical detection methods (for example Alamar Blue stain or Trypan Blue stain) for cytotoxic changes.

The supematants/cells were then harvested and sucked by means of a vacuum onto 96-well dot-blot chambers covered with a nylon membrane (in accordance with the manufacturer's information).

Cytotoxicity Determination

Substance-induced cytotoxic or cytostatic changes in the HepG2.2.15 cells were detected, for example, under the light microscope as changes in cell morphology. Such substance-induced changes in the HepG2.2.15 cells compared with untreated cells were visible, for example, as cytolysis, vacuolation or altered cell morphology. 50% cytotoxicity (Tox.-50) means that 50% of the cells show a morphology comparable to the corresponding cell control.

The tolerability of some of the compounds according to the invention was additionally tested on other host cells such as, for example, HeLa cells, primary human peripheral blood cells or transformed cell lines such as H-9 cells.

No cytotoxic changes were detectable with the test concentrations with antiviral activity of the compounds to be used according to the invention. As a rule, the compounds to be used according to the invention were tolerated up to 5 μM—and in some cases up to 25 μM.

Determination of the Antiviral Action

After the supernatants or lysed cells had been transferred to the nylon membrane of the blot apparatus (see above), the intra- or extracellular supernatants of the HepG2.2.15 cells were denatured (1.5 M NaCl/0.5 N NaOH), neutralized (3 M NaCl/0.5 M Tris HCl, pH 7.5) and washed (2× SSC). The DNA was then baked onto the membrane by incubating the filters at 120° C. for 2-4 hours.

DNA Hybridization

Detection of the viral DNA from the treated HepG2.2.15 cells on the nylon filters was easily carried out with non-radioactive, digoxigenin-labelled hepatitis B-specific DNA probes, each of which was labelled with digoxigenin, purified and employed for the hybridization in accordance with the manufacturer's information.

The prehybridization and hybridization took place in 5× SSC, 1× blocking reagent, 0.1% by weight N-lauroylsarcosine, 0.02% by weight SDS and 100 μg of herring sperm DNA. The prehybridization took place at 60° C. for 30minutes, and the specific hybridization with 20 to 40 ng/ml of the digoxigenized, denatured HBV-specific DNA took place at 60° C. for 14 hours. The filters were then washed.

Detection of HBV-DNA by Digoxigenin Antibodies

The immunological detection of the digoxigenin-labelled DNA took place in accordance with the manufacturer's information:

The filters were washed and prehybridized in a blocking reagent (in accordance with the manufacturer's information). Hybridization was then carried out with an anti-DIG antibody coupled to alkaline phosphatase for 30 minutes. After a washing step, the substrate of alkaline phosphatase, CSPD, was added, incubated with the filters for 5 minutes, then packed in plastic film and incubated at 37° C. for a further 15 minutes. The chemiluminescence of the hepatitis B-specific DNA signals was visualized by exposing the filters to an X-ray film (incubation depending on signal strength: 10 minutes to 2 hours).

The half-maximum inhibitory concentration (IC₅₀, 50% inhibitory concentration) was determined as the concentration at which the intra- or extracellular hepatitis B-specific band was reduced by the compound according to the invention by 50% compared with an untreated sample.

The results of the compounds of Examples 76, 82, 97, 99, 116 and 132 showed IC₅₀[μM] values of from 0.002 to 0.12 and Tox₅₀[μM] values of from 3 to 25.

Treatment of the hepatitis B virus-producing HepG2.2.15 cells with the compounds according to the invention surprisingly led to a reduction in intra- or extracellular viral DNA.

B(iii). Isoxazoles

### Example 1

N-(4-Fluoro-3-methylphenyl)-5-isopropyl-3-methylisoxazole-4-carboxamide

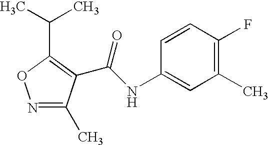

A solution of 10.97 g (69.3 mmol) of ethyl isobutyrylacetate and 4.93 g (69.3 mmol) of pyrrolidine in 50 ml of toluene is heated to reflux in an apparatus with a water trap for 3 hours. The toluene is then removed under reduced pressure, and the residue is dissolved in a mixture of 5.73 g (76.3 mmol) of nitroethane, 28 ml (201 mmol) of triethylamine and 120 ml of chloroform. This solution is cooled to 5° C. and a solution of 11.7 g (76.3 mmol) of phosphorus oxychloride in 20 ml of chloroform is added dropwise. After the addition is complete, the mixture is stirred at room temperature for 15 hours and then poured into 100 ml of ice-water. The organic phase is separated off, washed successively with 6 M hydrochloric acid, 5% strength sodium hydroxide solution, water and saturated aqueous NaCl solution and dried over sodium sulphate. Removal of the solvent by distillation and chromatography on silica gel (mobile phase dichloromethane) afford 7.52 g (55%) of ethyl 5-isopropyl-3-methylisoxazole-4-carboxylate as a colourless oil.

¹H-NMR (300 MHz, DMSO-D₆): 1.28 (d, 6H) ppm, 1.31 (t, 3H) ppm, 2.35 (s, 3H) ppm, 3.71 (quint., 1H) ppm, 4.27 (q, 2H) ppm.

A mixture of 7.5 g (38.0 mmol) of the ester, 70 ml of ethanol, 20 ml of water and 3.04 g (76.1 mmol) of sodium hydroxide is heated to reflux for 2 hours. After cooling, most of the ethanol is removed by distillation under reduced pressure. The aqueous phase is acidified with concentrated hydrochloric acid and then extracted several times with dichloromethane. The combined extracts are dried over sodium sulphate, and the solvent is removed. The residue is stirred with petroleum ether. 5.13 g (80%) of 5-isopropyl-3-methylisoxazole-4-carboxylic acid are isolated as a colourless solid by filtering off and drying under reduced pressure.

¹H-NMR (200 MHz, DMSO-D₆): 1.25 (d, 6H) ppm, 2.60 (s, 3H) ppm, 3.39 (quint., 1H) ppm.

MS (DCI/NH₃): 170 [M+H]⁺.

7.03 g (59.1 mmol) of thionyl chloride are added to 2 g (11.8 mmol) of the acid described. The mixture is heated to reflux with stirring until gas evolution ceases (about 1 hour). The thionyl chloride is removed under reduced pressure, and the resulting acid chloride (brown oil) is reacted further without purification.

A mixture of 56.3 mg (0.3 mmol) of the acid chloride, 37.5 mg (0.3 mmol) of 4-fluoro-3-methylaniline and 2.4 ml of 1,2-dichloroethane is mixed with 124 mg of morpholinomethyl-polystyrene (loading 3.69 mmol/g) and stirred at room temperature for 16 hours. The resin is filtered off and washed with dichloromethane. Removal of the volatile components under reduced pressure affords 80 mg (96%) of N-(4-fluoro-3-methylphenyl)-5-isopropyl-3-methylisoxazole-4-carboxamide as a colourless solid.

LC-MS (C18 column, 50×2.1 mm, 3.5 μm; gradient acetonitrile+0.1% formic acid [A], water+0.1% formic acid [B]: up to 4 min A/B=1:9, 4-6 min A/B=9:1; flow rate 0.5 ml/min; ionization ESI positive): Rt 4.3 min, m/z 276 [M]⁺.

### Example 2

N-(4-Fluoro-3-methylphenyl)-3,5-dimethylisoxazole-4-carboxamide

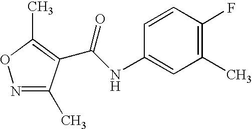

A solution of 2.53 g (18.8 mmol) of 4-fluoro-3-methylaniline and 2.88 ml (20.7 mmol) of triethylamine in 30 ml of dichloromethane is cooled to 0° C., and a solution of 3.0 g (18.8 nunol) of 3,5-dimethylisoxazolecarbonyl chloride in 10 ml of dichloromethane is added dropwise. The solution is stirred at 0° C. for 1 hour and then washed successively with 1 M hydrochloric acid, saturated aqueous sodium bicarbonate solution and saturated aqueous NaCl solution. The organic phase is dried over sodium sulphate and the volatile components are removed under reduced pressure. The remaining residue is chromatographed on silica gel (dichloromethane/ethyl acetate gradient). N-(4-Fluoro-3-methylphenyl)-3,5-dimethylisoxazole-4-carboxamide results as a colourless solid (4.0 g, 86%).

¹H-NMR (200 MHz, CDCl₃): 2.51 (s, 3H) ppm, 2.67 (s, 3H) ppm, 6.97 (t, 1H) ppm, 7.23 (m, 1H) ppm, 7.41 (m, 1H) ppm.

MS (DCI/NH₃): 249 (M+H)⁺.

### Example 3

N-(4-Fluoro-3-methylphenyl)-3,5-dimethylisoxazole-4-thiocarboxamide

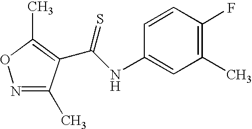

A mixture of 100 mg (0.40nimol) of N-(4-fluoro-3-methylphenyl)-3,5-dimethylisoxazole-4-carboxamide, 80 mg (0.20 mmol) of Lawesson's reagent and 5 ml of toluene is heated at 90° C. for 1 hour. Removal of the toluene by distillation under reduced pressure is followed by chromatography on silica gel (dichloromethane/ethyl acetate gradient). N-(4-Fluoro-3-methylphenyl)-3,5-dimethylisoxazole-4-thiocarboxamide results as a colourless solid (106 mg, 100%).

¹H-NMR (200 MHz, DMSO-D₆): 2.26 (s, 3H) ppm, 2.32 (s, 3H) ppm, 7.22 (t, 1H) ppm, 7.67 (m, 2H) ppm, 11.65 (s, br, 1H) ppm.

MS (DCI/NH₃): 265 (M+H)⁺.

The compounds in the following examples were synthesized in anlogy to Examples 1 to 3.

LCMS Methods:

Method A:

C18 column, 150×2.1 mm, 5 μm; gradient acetonitrile+0.1% formic acid [A], water+0.1% formic acid [B]: up to 9 min A/B=1:9, 9-10.1 min A/B=9:1; flow rate 0.5 ml/min; oven temperature 40° C., UV detection 210-350 mn, ionization ESI positive.

Method B:

C18 column, 50×2.1 mm, 3.5 μm; gradient acetonitrile+0.1% formic acid [A], water+0.1% formic acid [B]: up to 4 min A/B=1:9, 4-6 min A/B=9:1; flow rate 05 ml/min; oven temperature 40° C., WV detection 208-400 nm, ionization ESI positive.

Method C:

C18 column, 150×2.1 mm, 5 μm; gradient acetonitrile [A], 0.01 N hydrochloric acid [B], water [C]: up to 4 min A/B/C=10:45:45, 4-9 min A/B/C=90:5:5; flow rate 0.6 ml/min; oven temperature 40° C., UV detection 210 nm, ionization ESI positive.

| | | Retention | HPLC | 1H-NMR |
|---|---|---|---|---|
| Example | Structure | time (min.) | method | (200 MHz, DMSO) |
| 4 | 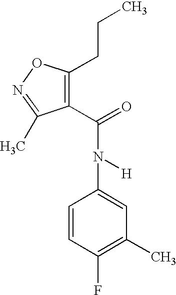 | 7.05 | A | |
| 5 | 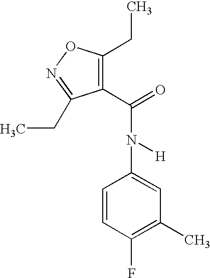 | 3.80 | B | |
| 6 | 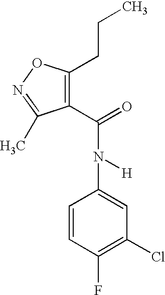 | 7.32 | A | |
| 7 | 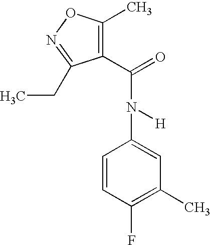 | 3.35 | B | |
| 8 | 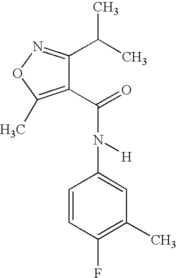 | 4.28 | B | |
| 9 | 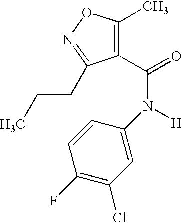 | 4.10 | B | |
| 10 | 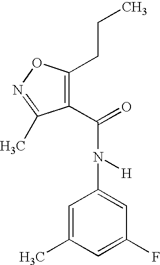 | 7.21 | A | |
| 11 | 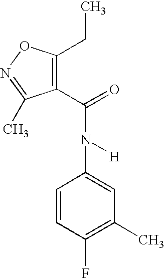 | 4.13 | B | |
| 12 | 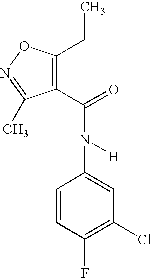 | 4.31 | B | |
| 13 | 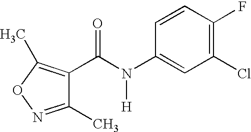 | 5.48 | C | |
| 14 | 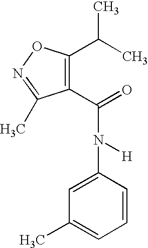 | 4.23 | B | |
| 15 | 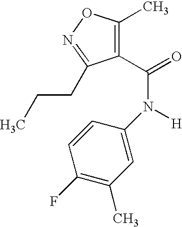 | 3.77 | B | |
| 16 | 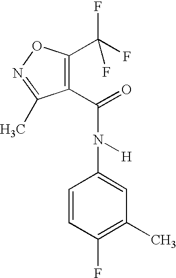 | 4.29 | B | |
| 17 | 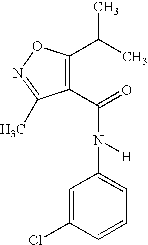 | 4.44 | B | |
| 18 | 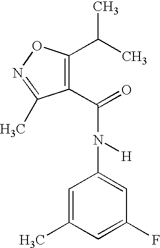 | 4.42 | B | |
| 19 | 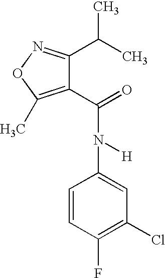 | 4.47 | B | |
| 20 | 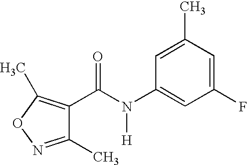 | 5.38 | C | |
| 21 | 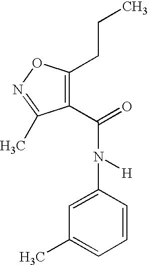 | 6.97 | A | |
| 22 | 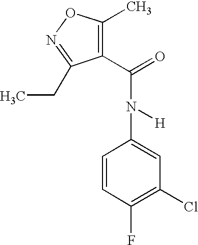 | 3.69 | B | |
| 23 | 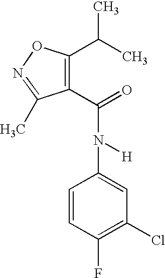 | 4.48 | B | |
| 24 | 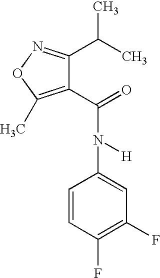 | 4.28 | B | |
| 25 | 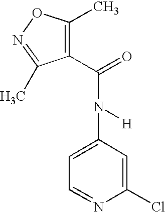 | 4.27 | B | |
| 26 | 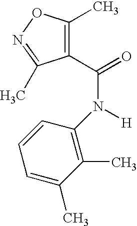 | 4.85 | B | |
| 27 | 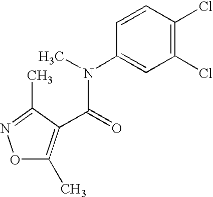 | | | 2.11(s, 3H), 2.14(s, 3H), 3.36(s, 3H), 7.23(dd, 1H), 7.59(d, 1H), 7.73(d, 1H). |
| 28 | 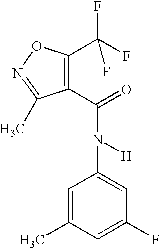 | 4.20 | B | |
| 29 | 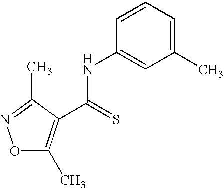 | | | 2.32(s, 3H), 2.33(s, 3H), 2.50(s, 3H), 7.11(d, 1H), 7.33(dd, 1H), 7.63(m, 2H), 11.68(s, br, 1H). |
| 30 | 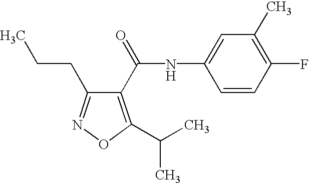 | | | (400 MHz, CD₂Cl₂) 1.00(t, 3H), 1.32(d, 6H), 1.71-1.83(m, 2H), 2.28(d, 3H), 2.87-2.95(m, 2H), 3.20-3.31(m, 1H), 6.97-7.03(m, 1H), 7.22(broad s, 1H), 7.29-7.36(m, 1H), 7.38-7.42(m, 1H). |
| 31 | 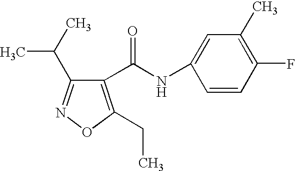 | | | (300 MHz, CDCl₃) 1.33-1.41(m, 9H), 2.29(d, 3H), 3.20-3.38(m, 1H), 3.99(q, 2H), 6.98(t, 1H), 7.15(broad s, 1H), 7.25-7.30(m, 1H), 7.41(dd, 1H). |

### Example 32

N-[(3,5-Dimethyl-4-isoxazolyl)methyl]-4-fluoro-3-methylaniline

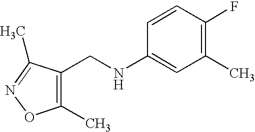

500 mg (2.01 mmol) of the compound from Example 2 are dissolved in 30 ml of tetrahydrofuran under argon and 0.65 g (8.56 mmol, 4.28 ml) of borane/dimethyl sulphide complex is added at 0° C. The mixture is then heated to boiling for 2 h. 4.13 ml of 1 N hydrochloric acid are added and the mixture is stirred under reflux for one further hour. Cooling to room temperature and addition of 12.5 ml of 0.5 M sodium hydroxide solution are followed by extraction with ethyl acetate and washing of the organic phase with saturated sodium chloride solution. It is dried over magnesium sulphate and the solvent is removed by distillation. The residue is then purified by chromatography on silica gel (1. dichloromethane, 2. cyclohexane:ethyl acetate 6:1) and recrystallized. The target compound is obtained in a yield of 54% (0.253 g).

MS (EIPOS): 234 [M+H]⁺

¹H-NMR (200 MHz, CDCl₃): δ=2.22 (d, 3H); 2.27 (s, 3H); 2.38 (s, 3H); 3.30 (broad s, 1H); 3.95 (s, 2H); 6.35-6.50 (m, 2H); 6.85 (t, 1H).

The compound of following Example 33 is prepared starting from the compound of Example 30 in analogy to the method of Example 32:

### Example 33

4-Fluoro-N-[(5-isopropyl-3-propyl-4-isoxazolyl)methyl]-3-methylaniline

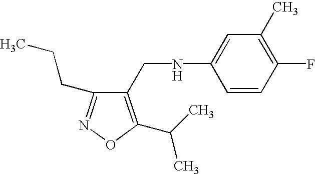

Yield: 13%

MS (DCI/NH₃)=291 [M+H]⁺, 308 [M+NH₄]⁺

¹H-NMR (300 MHz, CDCl₃): δ=0.95 (t, 3H); 1.33 (d, 6H); 1.60-1.80 (m, 2H); 2.21 (d, 3H); 2.70 (t, 2H); 2.95-3.11 (m, 1H); 3.22 (broad s, 1H); 3.95 (s, 2H); 6.37-6.50 (m, 2H); 6.86 (t, 1H).

### Example 34

Stage A:

3,5-Dimethyl-4-isoxazoleamine

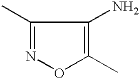

12.00 g (84.44 mmol) of 3,5-dimethyl-4-nitroisoxazole are introduced into 430 ml of water, and 106.15 g (1.984 mol) of ammonium chloride are added. At 4° C., 46.93 g (7.17 mol) of zinc are added over the course of 2 h, ethyl acetate is added to the reaction solution, and the organic phase is filtered through Celite. After drying over magnesium sulphate, the solvent is removed by distillation and the target compound is obtained in a yield of 86% (8.10 g).

¹H-NMR (300 MHz, CDCl₃): δ=2.20 (s, 3H); 2.28 (s, 3H); 2.51 (broad s, 2H).

Stage B:

N-(3,5-Dimethyl-4-isoxazolyl)-N′-(4-fluoro-3-methylphenyl)urea

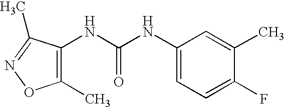

1.25 g (10.00 mmol) of 3-methyl-4-fluoroaniline are dissolved in 40 ml of dichloromethane, and 4.29 g (20.00 mmol) of 1,8-bis(dimethylamino)naphthalene are added. At 0° C., 0.72 ml (6.00 mmol) of trichloromethyl chloroformate in 10 ml of dichloromethane is added dropwise and stirred at room temperature for 1 h. The mixture is then diluted with 50 ml of dichloromethane and washed with ice-water, 1 N hydrochloric acid and saturated sodium bicarbonate solution. After drying over magnesium sulphate, the filtrate is mixed with 1.12 g (10.00 mmol) of the amine from stage A and heated to boiling for 4 h. The precipitate is filtered off with suction, washed with dichloromethane and recrystallized from ethanol. The target compound is obtained in a yield of 27% (0.71 g).

MS (DCI/NH₃): 264 [M+H]⁺

¹H-NMR (200 MHz, D₆-DMSO): δ=2.10 (s, 3H); 2.20 (d, 3H); 2.26 (s, 3H); 7.00 (t, 1H); 7.28-7.30 (m, 1H); 7.34 (dd, 1H); 7.68 (broad s, 1H); 8.72 (broad s, 1H).

### Example 35

N-(3,5-Dimethyl-4-isoxazolyl)-N′-(4-fluoro-3-methylphenyl)thiourea

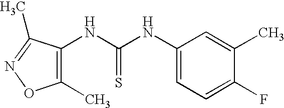

1.25 g (10.00 mmol) of 3-methyl-4-fluoroaniline are dissolved in 50 ml of toluene, and 2.18 g (11.00 mmol) of N,N′-thiocarbonyldiimidazole are added. The mixture is then heated to boiling for 45 minutes. After cooling to 50° C., 1.12 g (10.00 mmol) of the compound from Example 34 (stage A) are added, and the reaction solution is stirred at 70° C. for 4 h. The residue after removal of the solvent by distillation is stirred with ethyl acetate, and the crystals are filtered off with suction and recrystallized from ethanol. The target compound is obtained in a yield of 54% by weight (1.50 g).

MS (DCI/NH₃): 280 [M+H]⁺

¹H-NMR (200 MHz, D₆-DMSO): δ=2.10 (s, 3H); 2.20 (d, 3H); 2.25 (s, 3H); 7.05-7.18 (m, 1H); 7.18-7.38 (m, 2H); 8.95 (broad s, 1H); 9.80 (broad s, 1H).

HBV in Cell Culture; Testing for Combinatorial Activity

The antiviral action of the combinations according to the invention were investigated by methods based on those described by M. A. Sells et al., Proc. Natl. Acad. Sci. 84, 1005-1009 (1987) and B. E. Korba et al., Antiviral Research 19, 55-70 (1992) beschriebenen Methoden untersucht.

The tests in the combinatorial testing of the test substances were carried out by checkerboard titration.

The antiviral tests were carried out in 96-well microtitre plates. The first vertical row of the plate received only HepG2.2.15 cells in growth medium. It served as virus control.

Stock solutions of the test compounds (50 mM) were initially dissolved in DMSO; further dilutions were prepared in growth medium. The other wells contained the combinations according to the invention or their individual components in the test concentrations of, for example, 5 μM to 0.01 μM, starting from A2 to H11 of the 96-well microtitre plate.

Stock solutions of the substances to be tested were prepared on separate 96-well plates and then pipetted together with HepG2.2.15 cells onto the test plate. Test concentrations in the range from about 10-50 times above and below the IC-50 concentration were thus covered.

The test mixture was incubated at 37° C. and 5% CO₂ (v/v) for 8 days. On day 4, the medium was replaced by fresh inhibitor-containing medium.

Cytotoxicity Determination

Before harvesting the supematants/cell lysates for determining the antiviral effect, the HepG2.2.15 cells were examined under the light microscope or by means of biochemical detection methods (for example Alamar Blue stain or Trypan Blue stain) for cytotoxic changes.

Substance-induced cytotoxic or cytostatic changes in the HepG2.2.15 cells were detected, for example, under the light microscope as changes in cell morphology. Such substance-induced changes in the HepG2.2.15 cells compared with untreated cells were visible, for example, as cytolysis, vacuolation or altered cell morphology. 50% cytotoxicity (Tox.-50) means that 50% of the cells show a morphology comparable to the corresponding cell control.

The tolerability of some of the combinations according to the invention was additionally tested on other host cells such as, for example, HeLa cells, primary human peripheral blood cells or transformed cell lines such as H-9 cells.

No cytotoxic changes were detectable in the test concentration range.

Determination of the Antiviral Action

The supernatants/cell lysates were then harvested and sucked by reduced pressure onto 96-well dot-blot chambers covered with a nylon membrane (in accordance with the manufacturer's information).

In brief: after the supernatants or whole cell lysates had been transferred to the nylon membrane of the blot apparatus (see above), the nucleic acids present therein were denatured (1.5 M NaCl/0.5 N NaOH), neutralized (3 M NaCl/0.5 M Tris HCl, pH 7.5) and washed (2× SSC). The DNA was then baked onto the membranes by incubating the filters at 120° C. for 2-4 hours.

DNA Hybridization

Detection of the viral DNA from the treated HepG2.2.15 cells on the nylon filters was easily carried out with non-radioactive, digoxigenin-labelled hepatitis B-specific DNA probes, each of which was labelled with digoxigenin, purified and employed for the hybridization in accordance with the manufacturer's information.

Briefly: The prehybridization and hybridization took place in 5× SSC, 1 x blocking reagent, 0.1% by weight N-lauroylsarcosine, 0.02% by weight SDS and 100 μg of herring sperm DNA. The prehybridization took place at 60° C. for 30 minutes, and the specific hybridization with 20-40 ng/ml of the digoxigenized, denatured HBV-specific DNA took place at 60° C. for 14 hours. The filters were then washed.

Detection of HBV-DNA by Digoxigenin Antibodies

The immunological detection of the digoxigenin-labelled DNA took place in accordance with the manufacturer's information:

Briefly: The filters were washed and prehybridized in a blocking reagent (in accordance with the manufacturer's information). Hybridization was then carried out with an anti-DIG antibody coupled to alkaline phosphatase for 30 minutes. After a washing step, the substrate of alkaline phosphatase, CSPD, was added, incubated with the filters for 5 minutes, then packed in plastic film and incubated at 37° C. for a further 15 minutes. The chemiluminescence of the hepatitis B-specific DNA signals was visualized by an exposure of the filters by means of bioluminescence on an X-ray film or using a Lumi-Imager (incubation depending on the signal strength: about 2 minutes to about 2 hours), and the degree of blackening was measured.

The inhibitions were converted into % inhibitions according to the cutoff values from the internal test controls. To analyse the synergisitic activity of the combinations, the differences were found between calculated and measured inhibitions for each combination; compare Prichard et al., Antimicrob. Agents Chemother. 37, 540-545 (1993).

Treatment of HBV with the combinations according to the invention has a better antiviral action than single treatment; treatment of hepatitis B virus-producing HepG2.2.15 cells with the combinations according to the invention led to a greater reduction in intracellular viral DNA; the combination treatment shows synergistic activity.

1. Use of compounds of the formula 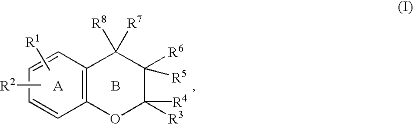
in which
R¹ denotes bromine, phenyl, pyrrolyl, pyridyl, pyrimidinyl, piperazinyl or quinolinyl, of which the cyclic substituents may each be substituted up to three times, identically or differently, by halogen, C₁-C₆-alkyl, trifluoromethyl, C₁-C₆-alkoxy, C₁-C₆-alkoxycarbonyl, C₁-C₆-alkylthio, nitro, cyano, amino, aminocarbonyl or benzyloxycarbonylamino,
R² denotes hydrogen or
R¹ and R² together with two adjacent carbon atoms of ring A denote a fused-on benzene ring,
R³ and R⁴ denote, independently of one another, linear or branched C₁-C₆-alkyl which may be substituted by carboxyl, C₁-C₄-alkoxy and/or C₁-C₄-alkoxycarbonyl, or
R³ and R⁴ together denote an optionally C₁-C₄-alkoxycarbonyl-substituted C₂-C₇-alkylene radical in which one CH₂ group may be replaced by an oxygen atom or by NR⁹ (with R⁹=hydrogen, benzoyl or benzyloxycarbonyl), or together with the carbon atom on which they are located denote a tetrahydro-2H-pyran ring,
R⁵ denotes linear or branched C₂-C₆-alkyl which may be substituted by cyano, C₁-C₄-alkoxycarbonyl, carboxyl or aminocarbonyl,
R⁶ denotes hydrogen or linear or branched C₂-C₆-alkyl which may be substituted by cyano, C₁-C₄-alkoxycarbonyl, carboxyl or aminocarbonyl,
R⁷ denotes hydrogen,
R⁸ denotes hydroxyl or
R⁷ and R⁸ together denote an oxo group,
for the production of antiviral medicaments.

2. Use of compounds I according to claim 1, in which
R¹ denotes phenyl, pyridyl or quinolinyl, each of which can be substituted up to three times by fluorine or chlorine or once by methyl, trifluoromethyl, methoxy, methylthio, nitro, cyano or amino; pyrrolyl which may be substituted by tert-butoxycarbonyl;
R² denotes hydrogen,
R³ and R⁴ each denotes methyl or
R³ and R⁴ together denote a C₃-C₅-alkylene radical in which one CH₂ group may be replaced by an oxygen atom, or together with the carbon atom on which they are located denote a tetrahydro-2H-pyran ring,
R⁵ denotes cyanoethyl,
R⁶ denotes hydrogen or cyanoethyl, and
R⁷ and R⁸ have the meanings indicated in claim 1.

3. Use of compounds I according to claim 1, in which
R¹ denotes phenyl which may be substituted up to three times by fluorine, up to twice by chlorine or once by methyl, trifluoromethyl, methoxy, methylthio, nitro, cyano or amino; pyridyl; pyrrolyl, which may be substituted by tert-butoxycarbonyl;
R² denotes hydrogen,
R³ and R⁴ each denotes methyl or
R³ and R⁴ together denote a C₃-C₅-alkylene radical or together with the carbon atom on which they are located denote a tetrahydro-2H-pyran ring,
R⁵ and R⁶ denote cyanoethyl, and
R⁷ and R⁸ have the meanings indicated in claim 1.

4. Use according to claims 1 to 3 for producing medicaments for the treatment and prophylaxis of hepatitis B.

5. Compounds of the formula I in claim 1, in which
R¹ denotes bromine, phenyl, pyrrolyl, pyridyl, pyrimidinyl, piperazinyl or quinolinyl, of which the cyclic substituents may in each case be substituted up to three times, identically or differently, by halogen, C₁-C₆-alkyl, trifluoromethyl, C₁-C₆-alkoxy, C₁-C₆-alkoxycarbonyl, C₁-C₆-alkylthio, nitro, cyano, amino, aminocarbonyl or benzyloxycarbonylamino, but where phenyl must have at least one substituent,
R² denotes hydrogen,
R³ and R⁴ denote, independently of one another, linear or branched C₁-C₆-alkyl which may be substituted by carboxyl, C₁-C₄-alkoxy and/or C₁-C₄-alkoxycarbonyl or
R³ and R⁴ together denote an optionally C₁-C₄-alkoxycarbonyl-substituted C₂-C₇-alkylene radical in which one CH₂ group may be replaced by an oxygen atom or by NR⁹ (with R⁹=hydrogen, benzoyl or benzyloxycarbonyl), or together with the carbon atom on which they are located denote a tetrahydro-2H-pyran ring,
R⁵ denotes linear or branched C₂-C₆-alkyl which may be substituted by cyano, C₁-C₄-alkoxycarbonyl, carboxyl or aminocarbonyl,
R⁶ denotes hydrogen or linear or branched C₂-C₆-alkyl which may be substituted by cyano, C₁-C₄-alkoxycarbonyl, carboxyl or aminocarbonyl,
R⁷ denotes hydrogen,
R⁸ denotes hydroxyl or
R⁷ and R⁸ together denote an oxo group.

6. Compounds according to claim 5, in which
R¹ denotes phenyl, pyrrolyl, pyridyl or quinolinyl, each of which may be substituted up to three times by fluorine or chlorine or once by methyl, trifluoromethyl, methoxy, methylthio, tert-butoxycarbonyl, nitro, cyano or amino, but where phenyl must have at least one substituent,
R² denotes hydrogen,
R³ and R⁴ each denotes methyl or
R³ and R⁴ together denote a C₃-C₅-alkylene radical in which one CH₂ group may be replaced by an oxygen atom, or together with the carbon atom on which they are located denote a tetrahydro-2H-pyran ring,
R⁵ denotes cyanoethyl,
R⁶ denotes hydrogen or cyanoethyl, and
R⁷ and R⁸ have the meanings indicated in claim 5.

7. Compounds according to claim 5, in which
R¹ denotes phenyl which may be substituted up to three times by fluorine, up to twice by chlorine or once by methyl, trifluoromethyl, methoxy, methylthio, nitro, cyano or amino, but where phenyl must have at least one substituent; pyridyl; pyrrolyl which may be substituted by tert-butoxycarbonyl;
R² denotes hydrogen,
R³ and R⁴ each denotes methyl or
R³ and R⁴ together denote a C₃-C₅-alkylene radical or together with the carbon atom on which they are located denote a tetrahydro-2H-pyran ring,
R⁵ and R⁶ denote cyanoethyl, and
R⁷ and R⁸ have the meanings indicated in claim 5.

8. Compounds of Examples 76, 82, 97, 99, 116 and 132.

9. Process for preparing the compounds according to claims 5 to 8, in which either
A) compounds of the formula 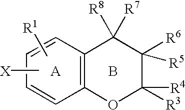
in which R¹ to R⁸ have the meanings indicated in claims 5 to 8, and X represents halogen, pseudohalogen, aryldiazonium salt or trifluoromethylsulphonate,
are subjected with compounds of the formula R²B(OH)₂ to a Suzuki reaction or are subjected with compounds of the formula R²SnR₃ (in which R² has the meaning indicated in claims 5 to 8, and R denotes alkyl) to a Stille or a Migita-Stille-Kosugi coupling, or
B) compounds of the formula 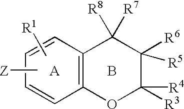
in which R¹ to R⁸ have the meanings indicated in claims 5 to 8, and Z represents —B(OH)₂ or —SnR₃, where R has the meaning indicated under A),
are reacted with compounds of the formula R²X where X has the meaning indicated under A), in a Stille or a Migita-Stille-Kosugi coupling, or
C) compounds of the formula 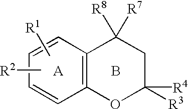
in which R¹ to R⁸ have the meanings indicated in claims 5 to 8, are reacted with α,β-unsaturated compounds suitable for introducing the substituents R⁵ and/or R⁶ in a Michael addition, and where appropriate
D) the reaction product from step A), B) or C) is hydrogenated to the corresponding chromanol.

10. Compounds according to claims 5 to 8 for controlling viral diseases.

11. Compounds according to claims 5 to 8 for controlling hepatitis B.

12. Medicament for viral diseases containing at least one compound according to claims 5 to 8 and, where appropriate, the pharmaceutical active substances.

13. Combinations of
A) at least one chromanone and/or chromanol,
B) at least one HBV antiviral active substance which is different from A and, where appropriate,
C) at least one immunomodulator.

14. Combinations according to claim 13 of
A) at least one chromanone and/or chromanol
B) (i) at least one HBV polymerase inhibitor, where appropriate in combination with an HBV-antiviral active substance which is different from A) and B(i), and, where appropriate,
C) at least oneimmunomodulator.

15. Combinations according to claims 13 and 14, whose component B contains at least one HBV DNA inhibitor or HBV core protein inhibitor.

16. Combinations according to claim 13 to 15, whose component A contains at least one compound of the formula 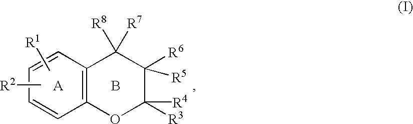
in which
R¹ denotes bromine, phenyl, pyrrolyl, pyridyl, pyrimidinyl, piperazinyl or quinolinyl, of which the cyclic substituents may each be substituted up to three times, identically or differently, by halogen, C₁-C₆-alkyl, trifluoromethyl, C₁-C₆-alkoxy, C₁-C₆-alkoxycarbonyl, C₁-C₆-alkylthio, nitro, cyano, amino, aminocarbonyl or benzyloxycarbonylamino,
R² denotes hydrogen or
R¹ and R² together with two adjacent carbon atoms of ring A denote a fused-on benzene ring,
R³ and R⁴ denote, independently of one another, linear or branched C₁-C₆-alkyl which may be substituted by carboxyl, C₁-C₄-alkoxy and/or C₁-C₄-alkoxycarbonyl, or
R³ and R⁴ together denote an optionally C₁-C₄-alkoxycarbonyl-substituted C₂-C₇-alkylene radical in which one CH₂ group may be replaced by an oxygen atom or by NR⁹ (with R⁹=hydrogen, benzoyl or benzyloxycarbonyl), or together with the carbon atom on which they are located denote a tetrahydro-2H-pyran ring,
R⁵ denotes linear or branched C₂-C₆-alkyl which may be substituted by cyano, C₁-C₄-alkoxycarbonyl, carboxyl or aminocarbonyl,
R⁶ denotes hydrogen or linear or branched C₂-C₆-alkyl which may be substituted by cyano, C₁-C₄-alkoxycarbonyl, carboxyl or aminocarbonyl,
R⁷ denotes hydrogen,
R⁸ denotes hydroxyl or
R⁷ and R⁸ together denote an oxo group.

17. Combinations according to claims 13 to 16, whose component A contains at least one compound of the formula I in claim 16, in which
R¹ denotes phenyl, pyridyl or quinolinyl, each of which can be substituted up to three times by fluorine or chlorine or once by methyl, trifluoromethyl, methoxy, methylthio, nitro, cyano or amino; pyrrolyl which may be substituted by tert-butoxycarbonyl;
R² denotes hydrogen,
R³ and R⁴ each denotes methyl or
R³ and R⁴ together denote a C₃-C₅-alkylene radical in which one CH₂ group may be replaced by an oxygen atom, or together with the carbon atom on which they are located denote a tetrahydro-2H-pyran ring,
R⁵ denotes cyanoethyl,
R⁶ denotes hydrogen or cyanoethyl, and
R⁷ and R⁸ have the meanings indicated above.

18. Combinations according to claims 13 to 17, whose component A contains at least one compound of the formula I in claim 16, in which
R¹ denotes phenyl which may be substituted up to three times by fluorine, up to twice by chlorine or once by methyl, trifluoromethyl, methoxy, methylthio, nitro, cyano or amino; pyridyl; pyrrolyl, which may be substituted by tert-butoxycarbonyl;
R² denotes hydrogen,
R³ and R⁴ each denotes methyl or
R³ and R⁴ together denote a C₃-C₅-alkylene radical or together with the carbon atom on which they are located denote a tetrahydro-2H-pyran ring,
R⁵ and R⁶ denote cyanoethyl, and
R⁷ and R⁸ have the meanings indicated above.

19. Combinations according to claims 13 to 18, whose component A contains at least one chromanone of Examples 76, 82, 97, 99, 116, 132.

20. Combinations according to claims 13 to 19, whose component B(i) contains lamivudine.

21. Combinations according to claims 13 to 20, whose component B(ii) contains at least one dihydropymidine of the formula 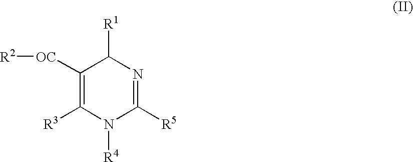
or its isomeric form 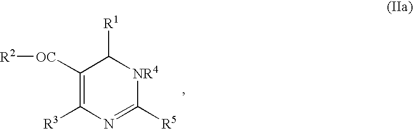
and/or their salts, in which
R¹ denotes phenyl, furyl, thienyl, triazolyl, pyridyl, cycloalkyl having 3 to 6 carbon atoms or radicals of the formulae 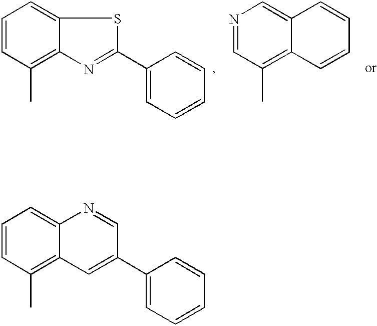
where the ring systems mentioned above are optionally substituted one or more times, identically or differently, by substituents selected from the group of halogen, trifluoromethyl, nitro, cyano, trifluoromethoxy, carboxyl, hydroxyl, C₁-C₆-alkoxy, C₁-C₆-alkoxycarbonyl and C₁-C₆-alkyl, where the alkyl radical in turn may be substituted by aryl having 6 to 10 carbon atoms or halogen,
and the mentioned ring systems are optionally substituted by —S—R⁶, —NR⁷R⁸, —CO—NR⁹R¹⁰, —SO₂—CF₃ and —A—CH₂—R¹¹, in which
R⁶ denotes optionally halogen-substituted phenyl,
R⁷ to R¹⁰ denote, independently of one another, hydrogen, phenyl, hydroxy-substituted phenyl, hydroxyl, C₁-C₆-acyl or C₁-C₆-alkyl, where the alkyl radical in turn may be substituted by hydroxyl, C₁-C₆-alkoxycarbonyl, phenyl or hydroxy-substituted phenyl,
A denotes a radical —O—, —S—, —SO— or —SO₂—,
R¹¹ denotes phenyl which is optionally substituted one or more times, identically or differently, by substituents selected from the group of halogen, nitro, trifluoromethyl, C₁-C₆-alkyl and C₁-C₆-alkoxy,
R² denotes a radical of the formulae —XR¹² or —NR¹³R¹⁴,
in which
X denotes a single bond or oxygen,
R¹² denotes hydrogen, straight-chain or branched C₁-C₆-alkoxycarbonyl, a straight-chain, branched or cyclic, saturated or unsaturated C₁-C₈-hydrocarbon radical which optionally contains one or two identical or different hetero chain members from the group of —O—, —CO—, —NH—, —N—(C₁-C₄-alkyl)—, —S— or —SO₂— and which is optionally substituted by halogen, nitro, cyano, hydroxyl, aryl having 6 to 10 carbon atoms, aralkyl having 6 to 10 carbon atoms, heteroaryl or a group of the formula
NR¹⁵R¹⁶,
in which R¹⁵ and R¹⁶ denote, independently of one another hydrogen, benzyl or C₁-C₆-alkyl,
R¹³ and R¹⁴ denote, independently of one another, hydrogen, C₁-C₆-alkyl or cycloalkyl having 3 to 6 carbon atoms,
R³ denotes hydrogen, amino or a radical of the formula 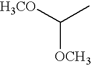
or
formyl, cyano, hydroxy-substituted C₁-C₆-alkylthio, trifluoromethyl or pyridyl or
a straight-chain, branched or cyclic, saturated or unsaturated hydrocarbon radical having up to 8 carbon atoms which is optionally substituted one or more times, identically or differently, by aryloxy having 6 to 10 carbon atoms, azido, halogen, cyano, hydroxyl, carboxyl, C₁-C₆-alkoxycarbonyl, a 5- to 7-membered heterocyclic ring, C₁-C₆-alkylthio or C₁-C₆-alkoxy (where the alkylthio or alkoxy radical may in turn be substituted by azido, amino, hydroxyl) and/or by the group —(CO)ₐ—NR¹⁷R¹⁸,
in which
a denotes zero or 1,
R¹⁷ and R¹⁸ denote, independently of one another, hydrogen or aryl having 6 to 10 carbon atoms, aralkyl having 6 to 10 carbon atoms or C₁-C₆-alkyl, each of which is optionally substituted by C₁-C₆-alkoxycarbonyl, amino, hydroxyl, phenyl or benzyl, where phenyl and benzyl are optionally substituted one or more times, identically or differently, by hydroxyl, carboxyl, C₁-C₆-alkyl or C₁-C₆-alkoxy, and/or C₁-C₆-alkyl is optionally substituted by —NH—CO—CH₃ or —NH—CO—CF₃, or
R17 and R¹⁸ together with the nitrogen atom on which they are located denote a morpholinyl, piperidinyl or pyrrolidinyl ring, or
R³ denotes optionally methoxy-substituted phenyl or
R² and R³ together denote a radical of the formula 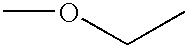
R⁴ denotes hydrogen, C₁-C₄-alkyl, C₂-C₄-alkenyl, benzoyl or acyl having 2 to 6 carbon atoms, preferably hydrogen, methyl, benzoyl or C₂-C₆-acyl, and
R⁵ denotes pyridyl, pyrimidyl or pyrazinyl, each of which may be substituted up to 3 times, identically or differently, by halogen, hydroxyl, cyano, trifluoromethyl, C₁-C₆-alkoxy, C₁-C₆-alkyl, C₁-C₆-alkylthio, carbalkoxy, C₁-C₆-acyloxy, amino, nitro, mono- or di-C₁-C₆-alkylamino.

22. Combinations according to claims 13 to 21, whose component B(ii) contains at least one compound of the formulae 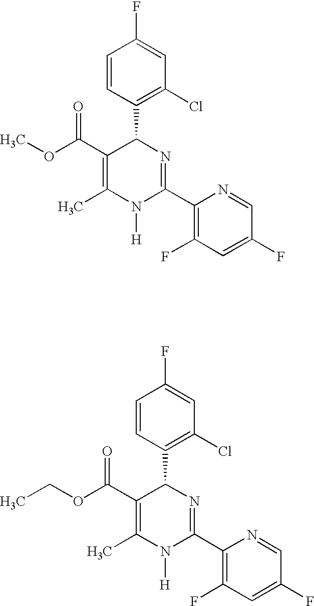
their isomeric forms and/or their salts.

23. Combinations according to claims 13 to 22, whose component B(ii) contains at least one compound of the formula 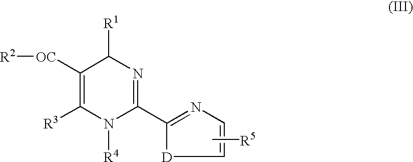
or its isomeric form 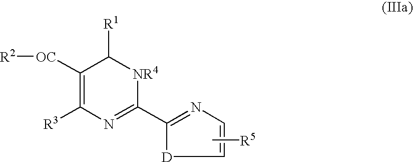
and/or their salts, in which
R¹ denotes phenyl, furyl, thienyl, pyridyl, cycloalkyl having 3 to 6 carbon atoms or a radical of the formulae 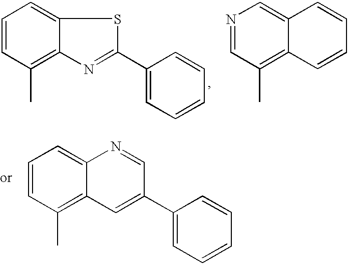
where the ring systems mentioned above are optionally substituted one or more times, identically or differently, by substituents selected from the group of halogen, trifluoromethyl, nitro, cyano, trifluoromethoxy, carboxyl, hydroxyl, C₁-C₆-alkoxy, C₁-C₆-alkoxycarbonyl and C₁-C₆-alkyl, where the alkyl radical in turn may be substituted by aryl having 6 to 10 carbon atoms or halogen,
and/or the mentioned ring systems are optionally substituted by groups of the formulae —S—R⁶, —NR⁷R⁸, —CO—NR⁹R¹⁰,
—SO₂—CF₃ and —A—CH₂—R¹¹,
in which
R⁶ denotes optionally halogen-substituted phenyl,
R⁷ to R¹⁰ denote, independently of one another, hydrogen, phenyl, hydroxy-substituted phenyl, hydroxyl, C₁-C₆-acyl or C₁-C₆-alkyl, where the alkyl radical in turn may be substituted by hydroxyl, C₁-C₆-alkoxycarbonyl, phenyl or hydroxy-substituted phenyl,
A denotes a radical —O—, —S—, —SO— or —SO₂—,
R¹¹ denotes phenyl which is optionally substituted one or more times, identically or differently, by substituents selected from the group of halogen, nitro, trifluoromethyl, C₁-C₆-alkyl and C₁-C₆-alkoxy,
R² denotes a radical of the formulae —OR¹² or —NR¹³R¹⁴,
in which
R¹² denotes hydrogen, C₁-C₆-alkoxycarbonyl or a straight-chain, branched or cyclic, saturated or unsaturated C₁-C₈-hydrocarbon radical which optionally contains one or two identical or different hetero chain members from the group of —O—, —CO—, —NH—, —N—(C₁-C₄-alkyl)—, —S— and —SO₂— and which is optionally substituted by halogen, nitro, cyano, hydroxyl, aryl having 6 to 10 carbon atoms or aralkyl having 6 to 10 carbon atoms, heteroaryl or a group of the formula —NR ⁵R¹⁶,
in which R¹⁵ and R¹⁶ denote, independently of one another, hydrogen, benzyl or C₁-C₆-alkyl,
R¹³ and R¹⁴ denote, independently of one another, hydrogen, C₁-C₆-alkyl or cycloalkyl having 3 to 6 carbon atoms,
R³ denotes hydrogen, amino or a radical of the formula 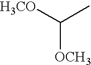
or formyl, cyano, hydroxy-substituted C₁-C₄-alkylthio, trifluoromethyl or
a straight-chain, branched or cyclic, saturated or unsaturated hydrocarbon radical having up to 8 carbon atoms, which is optionally substituted one or more times, identically or differently, by aryloxy having 6 to 10 carbon atoms, azido, cyano, hydroxyl, carboxyl, C₁-C₆-alkoxycarbonyl, a 5- to 7-membered heterocyclic ring, C₁-C₆-alkylthio or C₁-C₆-alkoxy (where the alkylthio or alkoxy radical in turn can be substituted by azido, amino or hydroxy) and/or by the group —(CO)ₐ—NR¹⁷R¹⁸,
in which a denotes zero or 1,
R¹⁷ and R¹⁸ denote, independently of one another, hydrogen or aryl, aralkyl having 6 to 10 carbon atoms or C₁-C₆-alkyl, which are optionally substituted by C₁-C₆-alkoxycarbonyl, amino, hydroxyl, phenyl or benzyl, where phenyl and benzyl are optionally substituted one or more times, identically or differently, by hydroxyl, carboxyl, C₁-C₆-alkyl or C₁-C₆-alkoxy, and/or C₁-C₆-alkyl is optionally substituted by —NH—CO—CH₃ or —NH—CO—CF₃, or
R¹⁷ and R¹⁸ together with the nitrogen atom on which they are located denote a morpholinyl, piperidinyl or pyrrolidinyl ring,
D denotes an oyxgen or sulphur atom and
R⁵ denotes hydrogen, halogen or straight-chain or branched alkyl having up to 6 carbon atoms.

24. Combinations according to claims 13 to 23, whose component B(iii) contains at least one compound of the formula 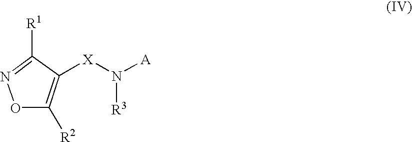
in which
R¹ and R² denote, independently of one another, alkyl which is optionally substituted by one or more halogen atoms,
X denotes a divalent radical from the series C═Y, —N(R⁴)—C(═Y)—, CH₂,
R³ and R⁴ denote, independently of one another, hydrogen or alkyl,
Y denotes an oxygen or sulphur atom and
A denotes aryl or hetaryl which are optionally substituted by 1 to 3 radicals selected, independently of one another, from the series halogen, alkyl, alkoxy, alkylthio, alkoxycarbonyl, aminocarbonylamino, mono- and dialkylamino, cyano, amino, mono- and dialkylaminocarbonyl.

25. Combinations according to claims 13 to 24, whose component B contains (i) an HBV polymerase inhibitor and/or (ii) a dihydropyrimidine.

26. Combinations according to claims 13 to 25, whose component B contains at least one compound of the formula 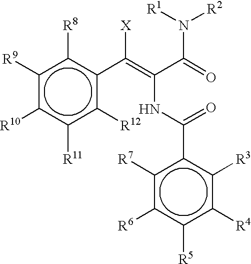
and/or its salt(s), in which
R¹ and R² denote, independently of one another, C₁-C₄-alkyl or, together with the nitrogen atom on which they are located, form a ring having 5 to 6 ring atoms which comprise carbon and/or oxygen,
R³-R¹²denote, independently of one another, hydrogen, halogen, C₁-C₄-alkyl, optionally substituted C₁-C₄-alkoxy, nitro, cyano or trifluoromethyl,
R¹³ denotes hydrogen, C₁-C₄-alkyl, C₁-C₇-acyl or aralkyl and
X denotes halogen or optionally substituted C₁-C₄-alkyl

27. Combinations according to claim 26, in which
X denotes chlorine, A denotes 1-piperidinyl and Y and Z each denotes phenyl.

28. Combinations according to claims 13 to 27, whose immunomodulator C contains interferons.

29. Combinations of A) at least one chromanone and/or chromanol, B)(i) lamivudine, ii) at least one dihydropyrimidine, (iii) at least one isoxazole and, where appropriate, (C) at least one interferon.

30. Process for producing the combinations according to claims 13 to 29, characterized in that the components A, B and, where appropriate, C are combined or prepared in a suitable way.

31. Combinations according to claims 13 to 29 for controlling diseases.

32. Medicament containing a combination according to claims 13 to 29 and at least one other pharmaceutical active substance and/or pharmaceutical excipient.

33. Use of combinations of claims 13 to 29 for producing a medicament for the treatment and prophylaxis of viral diseases.

34. Use of combinations of claims 13 to 29 for producing a medicament for the treatment and prophylaxis of hepatitis B infections.
